(19) United States
(12) Patent Application Publication
(10) Pub. No.: US 2010/0099681 A1
(43) Pub. Date: Apr. 22, 2010

(54) SUBSTITUTED 5-AMINOPYRAZOLES AND USE THEREOF

(75) Inventors: Lars Bärfacker, Oberhausen (DE); Raimund Kast, Wuppertal (DE); Nils Griebenow, Dormagen (DE); Heinrich Meier, Wuppertal (DE); Peter Kolkhof, Wuppertal (DE); Barbara Albrecht-Küpper, Wüelfrath (DE); Adam Nitsche, Pulheim (DE); Nicole Teusch, Wuelfrath (DE); Johannes-Peter Stasch, Solingen (DE); Dirk Schneider, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/540,657

(22) Filed: Aug. 13, 2009

(30) Foreign Application Priority Data
Aug. 21, 2008 (DE) 102008039083.6

(51) Int. Cl. A61K 31/498; A61K 31/517; A61K 31/437; A61K 31/4709; A61K 31/415; C07D 403/10; C07D 231/38; A61P 9/00

(52) U.S. Cl. 514/249; 514/266.23; 514/303; 514/314; 514/407; 544/353; 548/361.1; 548/371.4

(57) ABSTRACT

The present application relates to novel substituted 5-aminopyrazoles, methods of production thereof, use thereof alone or in combinations for the treatment and/or prophylaxis of diseases and use thereof for the production of medicinal products for the treatment and/or prophylaxis of diseases.

### RELATED APPLICATIONS/PATENTS AND INCORPORATION BY REFERENCE

This application claims priority to German Patent Application Number 102008039083.6 filed Aug. 21, 2008, the entire contents each of which is incorporated herein by reference. The foregoing application, and all documents cited therein and all documents cited or referenced therein, and all documents cited or referenced herein, including any U.S. or foreign patents or published patent applications, International patent applications, as well as, any non-patent literature references and any manufacturer's instructions, are hereby expressly incorporated herein by reference.

### BACKGROUND

1. Field of the Invention

The present application relates to novel substituted 5-aminopyrazoles, methods of production thereof, use thereof alone or in combinations for the treatment and/or prophylaxis of diseases and use thereof for the production of medicinal products for the treatment and/or prophylaxis of diseases.

2. Background of the Invention

Adenosine, a purine nucleoside, is present in all cells and is released under the action of a great many physiological and pathophysiological stimuli. Adenosine forms during the degradation of adenosine-5′-monophosphate (AMP) and S-adenosylhomocysteine and can be liberated from the cell via transporters or after cell damage. Extracellular adenosine can also arise via nucletidase-catalyzed degradation of adenine nucleotides. Then, by binding to specific receptors, the adenosine that has been released performs functions as a hormonelike substance or as a neurotransmitter. However, the concentration of extracellular adenosine fluctuates considerably and depends on the organ and the level of stress on the particular tissue. Thus, the extracellular concentration of adenosine increases dramatically in ischemic or hypoxic conditions. Then adenosine generally has cytoprotective functions, e.g. increasing the supply of oxygen or slowing down the metabolism of the organ in question.

The action of adenosine is mediated by specific receptors, which are subdivided into the four subtypes known so far: A1, A2a, A2b and A3. These receptors belong to the family of G protein-coupled receptors, which are characterized by seven transmembrane domains. Whereas the A1 and A3 receptors are coupled to Gi-proteins, which inhibit adenylate cyclase and therefore leads to a decrease of the intracellular cAMP content, the A2a and A2b receptors activate adenylate cyclase via Gs-proteins, which results in an increase in intracellular cAMP. Adenosine receptors can also be coupled to other signal transduction systems, e.g. phospholipase C. Thus, activation of the A1 receptors can also lead to stimulation of potassium channels or inhibition of calcium channels.

“Adenosine receptor-selective ligands” means, according to the invention, substances that bind selectively to one or more subtypes of the adenosine receptors and in so doing either imitate the action of adenosine (adenosine agonists) or block its action (adenosine antagonists).

The aforesaid receptor selectivity can be determined through the action of the substances on cell lines, which after stable transfection with the corresponding cDNA express the respective receptor subtypes (Olah M. E, Ren H., Ostrowski J., Jacobson K. A. and Stiles G. L.: Cloning, expression, and characterization of the unique bovine A1 adenosine receptor. Studies on the ligand binding site by site-directed mutagenesis, J. Biol. Chem. 1992, 267, 10764-10770, the disclosure of which is hereby incorporated by reference in its entirety).

The action of the substances on such cell lines can be detected by biochemical measurement of the intracellular messenger cAMP (Klotz N., Hessling J., Hegler J., Owman C., Kull B., Fredholm B. B. and Lohse J. M.: Comparative pharmacology of human adenosine receptor subtypes—characterization of stably transfected receptors in CHO cells., Naunyn Schmiedebergs Arch. Pharmacol. 1998, 357, 1-9, the disclosure of which is hereby incorporated by reference in its entirety).

Selective interaction with the adenosine receptor subtypes offers a broad spectrum of therapeutic potential, for example regulation of heart rate, contractile force and blood pressure in the cardiovascular system, regulation of renal function and of the respiratory system, of the immune system and effects on some functions of the central nervous system and of cell growth (Jacobson K. A and Gao Z., Adenosine receptors as therapeutic targets, Nature Reviews Drug Discovery 2006, 5, 247-264).

Adenosine A1 receptors are strongly expressed in the brain (e.g. cortex, hippocampus), but also in peripheral organs and tissues such as heart, kidney, lung or adipocytes.

In the kidney, adenosine A1 receptors are involved decisively in the control of fluid and electrolyte balance. A1 receptors are expressed in the preglomerular microcirculation, in the glomerulus, in the juxtaglomerular apparatus, and in the collecting tube and Henle's loop. Activation of the A1 receptors in the kidney causes the glomerular filtration rate and the renal blood flow to decrease. These effects are brought about by vasoconstriction of the afferent arterioles and by suppression of the tubuloglomerular feedback mechanism (TGF), which is mainly responsible for the autoregulation of the glomerular vascular resistance (Welch J. W., Current Opinion in Pharmacology 2002, 2, 165-170).

It was shown in various animal studies and human clinical trials that the inhibition of the A1 receptors by selective A1 antagonists is uricosuric, natriuretic and potassium-sparing and diuretic. Furthermore, the glomerular filtration rate was unaffected by adenosine A1 receptor antagonists (Vallon V., Miracle C. and Thomson S., Adenosine and kidney function: potential implications in patients with heart failure, Eur. J. Heart Failure 2008, 10, 176-187). The renal-protective action of the A1 antagonists, by maintaining the glomerular filtration rate and the renal plasma flow, could also be demonstrated in various animal models of acute and chronic renal failure (Welch J. W., Current Opinion in Pharmacology 2002, 2, 165-170; Nagashima K., Kusaka H. and Karasawa A., Protective effects of KW-3902, an adenosine A1 receptor antagonist, against cisplatin-induced acute renal failure in rats. Jpn. J. Pharmacol. 1995, 67, 349-357; Kalk P., Eggert B., Relle K., Godes M., Heiden S., Sharkovska Y., Fischer Y., Ziegler D., Bielenberg G. W. and Hocher B.: The adenosine A1 receptor antagonist SLV 320 reduces myocardial fibrosis in rats with 5/6 nephrectomy without affecting blood pressure. Brit. J. Pharmacol. 2007, 151, 1025-1032).

Decrease in renal function is often observed in patients with heart failure. Treatment is with loop diuretics, for example furosemide, but this leads to a decrease in glomerular filtration rate, and therefore leads to an undesirable complication of the condition of patients with heart failure. Furthermore, diuretic resistance is another indicator for a poor prognosis for patients with heart failure.

Selective A1 antagonists are therefore suitable inter alia for the treatment of acute decompensated heart failure and chronic heart failure. In addition they can be used for renal protection in nephropathy and other kidney diseases, for example acute and chronic renal failure and chronic renal insufficiency.

Substituted 5-aminopyrazoles of various kinds for the treatment of diabetes are disclosed in WO 2004/050651, WO 2005/086656, WO 2005/112923 and WO 2007/027842. WO 93/19054 describes arylaminopyrazoles as fungicides. Pyridyloxypyrazoles are claimed as herbicides in JP 07-285962. WO 2008/008286 discloses substituted pyrazoles as ghrelin receptor antagonists for the treatment of obesity.

The problem facing the present invention is the provision of novel compounds that act as potent and selective antagonists of the adenosine A1 receptor and as such are suitable for the treatment and/or prophylaxis of diseases.

### SUMMARY OF THE INVENTION

The present invention relates to compounds of general formula (I)

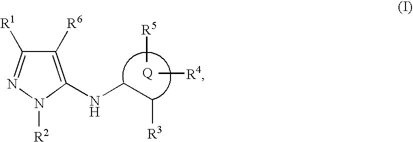

in which
Q stands for phenyl or pyridyl,
R¹ stands for hydrogen, cyano, (C₁-C₃)-alkyl, trifluoromethyl, (C₁-C₃)-alkoxy or trifluoromethoxy,
R² stands for phenyl, naphthyl or 5- or 6-membered heteroaryl,
   where phenyl, naphthyl and 5- or 6-membered heteroaryl can be substituted with 1 or 2 substituents selected independently of one another from the group comprising halogen, cyano, (C₁-C₄)-alkyl, monofluoromethyl, difluoromethyl, trifluoromethyl, (C₁-C₄)-alkoxy, monofluoromethoxy, difluoromethoxy and trifluoromethoxy,
R³ stands for hydroxycarbonyl, aminocarbonyl, cyanoaminocarbonyl, (C₁-C₄)-alkylsulfonylaminocarbonyl, oxadiazolonyl or tetrazol-5-yl,
   where oxadiazolonyl can be substituted with a methyl substituent,
R⁴ stands for hydrogen, halogen, (C₁-C₄)-alkyl, monofluoromethyl, difluoromethyl, trifluoromethyl, (C₁-C₄)-alkoxy, monofluoromethoxy, difluoromethoxy or trifluoromethoxy,
R⁵ stands for hydrogen, halogen, (C₁-C₄)-alkyl, monofluoromethyl, difluoromethyl, trifluoromethyl, (C₁-C₄)-alkoxy, monofluoromethoxy, difluoromethoxy or trifluoromethoxy,
R⁶ stands for a group of formula

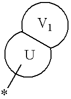

where
* denotes the site of attachment to the pyrazole,
ring U stands for phenyl, pyridyl, pyrimidinyl or pyrazinyl,
   in which phenyl, pyridyl, pyrimidinyl and pyrazinyl can be substituted with 1 to 3 substituents selected independently of one another from the group comprising halogen, (C₁-C₄)-alkyl, trifluoromethyl, hydroxy, (C₁-C₄)-alkoxy and trifluoromethoxy,
      in which (C₁-C₄)-alkyl and (C₁-C₄)-alkoxy for their part can be substituted with 1 or 2 substituents selected independently of one another from the group comprising hydroxy and (C₁-C₄)-alkoxy,
and
ring V₁ stands for a phenyl ring fused to ring U or a 5- or 6-membered heteroaryl ring fused to ring U,
   in which the phenyl ring and the 5- or 6-membered heteroaryl ring can be substituted with 1 to 4 substituents selected independently of one another from the group comprising halogen, cyano, (C₁-C₄)-alkyl, trifluoromethyl, (C₁-C₄)-alkoxy, trifluoromethoxy, (C₁-C₄)-alkylcarbonyl, amino, mono-(C₁-C₄)-alkylamino and di-(C₁-C₄)-alkylamino,
      and their salts, solvates and solvates of the salts.

### DETAILED DESCRIPTION OF THE INVENTION

Compounds according to the invention are the compounds of formula (I) and their salts, solvates and solvates of the salts, the compounds of the formulas stated below that are covered by formula (I) and their salts, solvates and solvates of the salts and the compounds covered by formula (I), and stated subsequently as examples of application and their salts, solvates and solvates of the salts, unless the compounds covered by formula (I), stated subsequently, are already salts, solvates and solvates of the salts.

Depending on their structure, the compounds according to the invention can exist in stereoisomeric forms (enantiomers, diastereomers). The present invention therefore includes the enantiomers or diastereomers and respective mixtures thereof. The stereoisomerically uniform constituents can be isolated in a known manner from said mixtures of enantiomers and/or diastereomers.

If the compounds according to the invention can occur in tautomeric forms, the present invention comprises all tautomeric forms.

Physiologically harmless salts of the compounds according to the invention are preferred as salts within the scope of the present invention. It also comprises salts that are not suitable themselves for pharmaceutical applications, but can be used for example for the isolation or purification of the compounds according to the invention.

Physiologically harmless salts of the compounds according to the invention comprise salts of acid addition of inorganic acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically harmless salts of the compounds according to the invention also comprise salts of the usual bases, for example and preferably alkali metal salts (e.g. sodium and potassium salts), alkaline-earth salts (e.g. calcium and magnesium salts) and ammonium salts, derived from ammonia or organic amines with 1 to 16 carbon atoms, for example and preferably ethyl amine, diethylamine, triethylamine, ethyldiisopropylamine, monoethanolamine, diethanolamine, trisethanolamine, dicyclohexyl amine, dimethylaminoethanol, procaine, dibenzylamine, N-methylmorpholine, arginine, lysine, ethylenediamine and N-methylpiperidine.

Those forms of the compounds according to the invention that form a complex in the solid or liquid state by coordination with solvent molecules are designated as solvates within the scope of the invention. Hydrates are a special form of solvates, where coordination takes place with water. Hydrates are preferred as solvates within the scope of the present invention.

Moreover, the present invention also comprises prodrugs of the compounds according to the invention. The term “prodrugs” includes compounds that can themselves be biologically active or inactive, but during the time that they are inside the body they are converted (for example metabolically or hydrolytically) to compounds according to the invention.

Within the scope of the present invention, unless specified otherwise, the substituents have the following meanings:

Alkyl stands within the scope of the invention for a linear or branched alkyl residue with 1 to 4 or 1 to 3 carbon atoms. As examples, and preferably, we may mention: methyl, ethyl, n-propyl, isopropyl, n-butyl, iso-butyl, sec.-butyl and tert.-butyl.

Alkylcarbonyl stands within the scope of the invention for a linear or branched alkyl residue with 1 to 4 carbon atoms and a carbonyl group attached in position 1. As examples, and preferably, we may mention: methylcarbonyl, ethylcarbonyl, n-propylcarbonyl, iso-propylcarbonyl, n-butylcarbonyl, iso-butylcarbonyl and tert.-butylcarbonyl.

Alkoxy stands within the scope of the invention for a linear or branched alkoxy residue with 1 to 4 or 1 to 3 carbon atoms. As examples, and preferably, we may mention: methoxy, ethoxy, n-propoxy, isopropoxy, n-butoxy and tert.-butoxy.

Mono-alkylamino stands within the scope of the invention for an amino group with a linear or branched alkyl substituent that has 1 to 4 carbon atoms. As examples, and preferably, we may mention: methylamino, ethylamino, n-propylamino, isopropylamino, n-butylamino and tert.-butylamino.

Di-alkylamino stands within the scope of the invention for an amino group with two identical or different linear or branched alkyl substituents, each with 1 to 4 carbon atoms. As examples, and preferably, we may mention: N,N-dimethylamino, N,N-diethylamino, N-ethyl-N-methylamino, N-methyl-N-n-propylamino, N-isopropyl-N-n-propylamino, N,N-diisopropylamino, N-n-butyl-N-methylamino and N-tert.-butyl-N-methylamino.

Alkylsulfonylaminocarbonyl stands within the scope of the invention for an amino group that is attached via a carbonyl group, and bears a linear or branched alkylsulfonyl substituent with 1 to 4 carbon atoms joined via the sulfonyl group to the N-atom. As examples, and preferably, we may mention: methylsulfonylaminocarbonyl, ethylsulfonylaminocarbonyl, n-propylsulfonylaminocarbonyl, isopropylsulfonylaminocarbonyl, n-butylsulfonylaminocarbonyl and tert.-butylsulfonylaminocarbonyl.

Heteroaryl stands within the scope of the invention for a monocyclic aromatic heterocycle (heteroaromatic), fused to ring U, with 5 or 6 ring atoms in total, and containing up to three identical or different ring heteroatoms from the group N, O and/or S. As examples we may mention: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl. Monocyclic 5- or 6-membered heteroaryl residues with up to two ring heteroatoms from the group N, O and/or S are preferred, for example furyl, thienyl, thiazolyl, oxazolyl, isothiazolyl, isoxazolyl, pyrazolyl, imidazolyl, pyridyl, pyrimidinyl, pyridazinyl, pyrazinyl.

Halogen includes, within the scope of the invention, fluorine, chlorine, bromine and iodine. Fluorine and chlorine are preferred.

In the formulas of the group, for which R², R⁶ or Q can stand, the end point of the line where there is a symbol ##, * or #, does not stand for a carbon atom or a CH₂ group, but is a constituent of the bond to the atom designated in each case, to which R², R⁶ or the amino group is bound.

If residues in the compounds according to the invention are substituted, unless specified otherwise, the residues can be substituted once or more than once. Within the scope of the present invention, for all residues that occur more than once, their meaning is independent of one another. Substitution with one, two or three identical or different substituents is preferred. Substitution with one substituent is quite especially preferred.

Preferred, within the scope of the present invention, are compounds of formula (I) in which
Q stands for phenyl or pyridyl,
R¹ stands for hydrogen, cyano, (C₁-C₃)-alkyl, trifluoromethyl, (C₁-C₃)-alkoxy or trifluoromethoxy,
R² stands for phenyl, naphthyl or 5- or 6-membered heteroaryl,
   where phenyl, naphthyl and 5- or 6-membered heteroaryl can be substituted with 1 or 2 substituents selected independently of one another from the group comprising halogen, cyano, (C₁-C₄)-alkyl, trifluoromethyl, (C₁-C₄)-alkoxy and trifluoromethoxy,
R³ stands for hydroxycarbonyl, aminocarbonyl, cyanoaminocarbonyl, (C₁-C₄)-alkylsulfonylaminocarbonyl, oxadiazolonyl or tetrazol-5-yl,
   where oxadiazolonyl can be substituted with a methyl substituent,
R⁴ stands for hydrogen, halogen, (C₁-C₄)-alkyl, difluoromethyl, trifluoromethyl, (C₁-C₄)-alkoxy, difluoromethoxy or trifluoromethoxy,
R⁵ stands for hydrogen, halogen, (C₁-C₄)-alkyl, difluoromethyl, trifluoromethyl, (C₁-C₄)-alkoxy, difluoromethoxy or trifluoromethoxy,
R⁶ stands for a group of formula

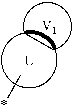

where
* denotes the site of attachment to the pyrazole,
ring U stands for phenyl, pyridyl, pyrimidinyl or pyrazinyl,
   in which phenyl, pyridyl, pyrimidinyl and pyrazinyl can be substituted with 1 to 3 substituents selected independently of one another from the group comprising halogen and (C₁-C₄)-alkyl,
and
ring V₁ stands for a phenyl ring fused to ring U or a 5- or 6-membered heteroaryl ring fused to ring U,
   in which the phenyl ring and the 5- or 6-membered heteroaryl ring can be substituted with 1 to 4 substituents selected independently of one another from the group comprising halogen, cyano, (C₁-C₄)-alkyl, trifluoromethyl, (C₁-C₄)-alkoxy, (C₁-C₄)-alkylcarbonyl, amino, mono-(C₁-C₄)-alkylamino and di-(C₁-C₄)-alkylamino,
      and their salts, solvates and solvates of the salts.

Compounds of formula (I) are also preferred within the scope of the present invention, in which
Q stands for phenyl,
R¹ stands for hydrogen, methyl or trifluoromethyl, R² stands for phenyl,
   where phenyl can be substituted with 1 or 2 substituents selected independently of one another from the group comprising fluorine, chlorine, methyl, ethyl, trifluoromethyl, methoxy, ethoxy and trifluoromethoxy,
R³ stands for hydroxycarbonyl,
R⁴ stands for hydrogen, fluorine, chlorine, methyl, ethyl, difluoromethyl, trifluoromethyl, methoxy, ethoxy, difluoromethoxy or trifluoromethoxy,
R⁵ stands for hydrogen,
R⁶ stands for a group of formula

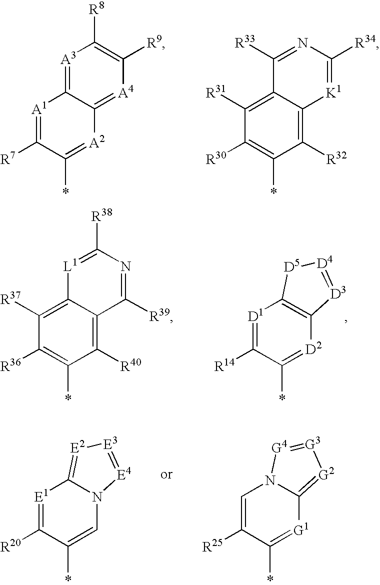

where
* denotes the site of attachment to the pyrazole,
A¹ stands for CR¹⁰ or N,
   in which
   R¹⁰ stands for hydrogen, fluorine, chlorine or methyl,
A² stands for CR¹¹ or N,
   in which
   R¹¹ stands for hydrogen, fluorine, chlorine or methyl,
A³ stands for CR¹² or N,
   in which
   R¹² stands for hydrogen, fluorine, chlorine, methyl or methoxy,
A⁴ stands for CR¹³ or N,
   in which
   R¹³ stands for hydrogen, fluorine, chlorine, methyl or methoxy,
D¹ stands for CR¹⁵ or N,
   in which
   R¹⁵ stands for hydrogen, fluorine, chlorine or methyl,
D² stands for CR¹⁶ or N,
   in which
   R¹⁶ stands for hydrogen, fluorine, chlorine or methyl,
D³ stands for CR¹⁷ or N,
   in which
   R¹⁷ stands for hydrogen, fluorine, chlorine or methyl,
D⁴ stands for CR¹⁸ or N,
   in which
   R¹⁸ stands for hydrogen, fluorine, chlorine or methyl,
D⁵ stands for NR¹⁹, O or S,
   in which
   R¹⁹ stands for hydrogen or methyl,
with the proviso that at least one of the groups D¹, D², D³, D⁴ and D⁵ stands for N or NR¹⁹,
E¹ stands for CR²¹ or N,
   in which
   R²¹ stands for hydrogen, fluorine, chlorine or methyl,
E² stands for CR²² or N,
   in which
   R²² stands for hydrogen, fluorine, chlorine or methyl,
E³ stands for CR²³ or N,
   in which
   R²³ stands for hydrogen, fluorine, chlorine, methyl or amino,
E⁴ stands for CR²⁴ or N,
   in which
   R²⁴ stands for hydrogen, fluorine, chlorine or methyl,
with the proviso that at most 2 of the groups E², E³ and E⁴ stand for N,
G¹ stands for CR²⁶ or N,
   in which
   R²⁶ stands for hydrogen, fluorine, chlorine or methyl,
G² stands for CR²⁷ or N,
   in which
   R²⁷ stands for hydrogen, fluorine, chlorine or methyl,
G³ stands for CR²⁸ or N,
   in which
   R²⁸ stands for hydrogen, fluorine, chlorine, methyl or amino,
G⁴ stands for CR²⁹ or N,
   in which
   R²⁹ stands for hydrogen, fluorine, chlorine or methyl,
with the proviso that at most 2 of the groups G², G³ and G⁴ stand for N,
K¹ stands for CR³⁵ or N,
   in which
   R³⁵ stands for hydrogen, fluorine, chlorine or methyl,
L¹ stands for CR⁴¹ or N,
   in which
   R⁴¹ stands for hydrogen, fluorine, chlorine or methyl,
R⁷ stands for hydrogen, fluorine, chlorine, methyl, hydroxy, methoxy or ethoxy,
R⁸ stands for hydrogen, fluorine, chlorine, methyl or methoxy,
R⁹ stands for hydrogen, fluorine, chlorine, methyl, methoxy, amino, methylamino or dimethylamino,
R¹⁴ stands for hydrogen, fluorine, chlorine, methyl, hydroxy, methoxy or ethoxy,
R²⁰ stands for hydrogen, fluorine, chlorine, methyl, hydroxy, methoxy or ethoxy,
R²⁵ stands for hydrogen, fluorine, chlorine, methyl, hydroxy, methoxy or
R³⁹ stands for hydrogen, fluorine, chlorine, methyl, hydroxy, methoxy or ethoxy,
R³¹ stands for hydrogen, fluorine, chlorine or methyl, ethoxy,
R³² stands for hydrogen, fluorine, chlorine or methyl,
R³³ stands for hydrogen, fluorine, chlorine, methyl or amino,
R³⁴ stands for hydrogen, fluorine, chlorine, methyl, methoxy, amino, methylamino or dimethylamino,
R³⁶ stands for hydrogen, fluorine, chlorine, methyl, hydroxy, methoxy or
R³⁷ stands for hydrogen, fluorine, chlorine or methyl,
R³⁸ stands for hydrogen, fluorine, chlorine, methyl, methoxy, amino, methylamino or dimethylamino,
R³⁹ stands for hydrogen, fluorine, chlorine, methyl or amino,
and
R⁴⁰ stands for hydrogen, fluorine, chlorine or methyl,
   and their salts, solvates and solvates of the salts.

Within the scope of the present invention, compounds of formula (I) are also preferred in which
Q stands for phenyl,
R¹ stands for hydrogen, cyano, methyl, ethyl or trifluoromethyl,
R² stands for phenyl,
   where phenyl can be substituted with 1 or 2 substituents selected independently of one another from the group comprising fluorine, chlorine, (C₁-C₄)-alkyl, trifluoromethyl, (C₁-C₄)-alkoxy and trifluoromethoxy,
R³ stands for hydroxycarbonyl or methylsulfonylaminocarbonyl,
R⁴ stands for hydrogen, fluorine, chlorine, methyl, ethyl, difluoromethyl, trifluoromethyl, methoxy, ethoxy, difluoromethoxy or trifluoromethoxy,
R⁵ stands for hydrogen, fluorine, chlorine, methyl, ethyl, difluoromethyl, trifluoromethyl, methoxy, ethoxy, difluoromethoxy or trifluoromethoxy,
R⁶ stands for a group of formula

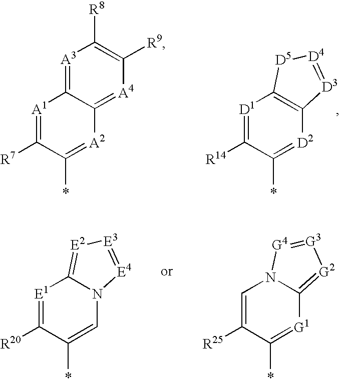

where
* denotes the site of attachment to the pyrazole,
A¹ stands for CR¹⁰ or N,
   in which
   R¹⁰ stands for hydrogen, fluorine, chlorine or methyl,
A² stands for CR^{H} or N,
   in which
   R¹¹ stands for hydrogen, fluorine, chlorine or methyl,
A³ stands for CR¹² or N,
   in which
   R¹² stands for hydrogen, fluorine, chlorine or methyl,
A⁴ stands for CR¹³ or N,
   in which
   R¹³ stands for hydrogen, fluorine, chlorine or methyl,
D¹ stands for CR¹⁵ or N,
   in which
   R¹⁵ stands for hydrogen, fluorine, chlorine or methyl,
D² stands for CR¹⁶ or N,
   in which
   R¹⁶ stands for hydrogen, fluorine, chlorine or methyl,
D³ stands for CR¹⁷ or N,
   in which
   R¹⁷ stands for hydrogen, fluorine, chlorine or methyl,
D⁴ stands for CR¹⁸ or N,
   in which
   R¹⁸ stands for hydrogen, fluorine, chlorine or methyl,
D⁵ stands for NR¹⁹, O or S,
   in which
   R¹⁹ stands for hydrogen or methyl,
with the proviso that at least one of the groups D¹, D², D³, D⁴ and D⁵ stands for N or NR¹⁹,
E¹ stands for CR²¹ or N,
   in which
   R²¹ stands for hydrogen, fluorine, chlorine or methyl,
E² stands for CR²² or N,
   in which
   R²² stands for hydrogen, fluorine, chlorine or methyl,
E³ stands for CR²³ or N,
   in which
   R²³ stands for hydrogen, fluorine, chlorine or methyl,
E⁴ stands for CR²⁴ or N,
   in which
   R²⁴ stands for hydrogen, fluorine, chlorine or methyl,
with the proviso that at most 2 of the groups E², E³ and E⁴ stand for N,
G¹ stands for CR²⁶ or N,
   in which
   R²⁶ stands for hydrogen, fluorine, chlorine or methyl,
G² stands for CR²⁷ or N,
   in which
   R²⁷ stands for hydrogen, fluorine, chlorine or methyl,
G³ stands for CR²⁸ or N,
   in which
   R²⁸ stands for hydrogen, fluorine, chlorine or methyl,
G⁴ stands for CR²⁹ or N,
   in which
   R²⁹ stands for hydrogen, fluorine, chlorine or methyl,
with the proviso that at most 2 of the groups G², G³ and G⁴ stand for N,
R⁷ stands for hydrogen, fluorine, chlorine or methyl,
R⁸ stands for hydrogen, fluorine, chlorine or methyl,
R⁹ stands for hydrogen, fluorine, chlorine or methyl,
R¹⁴ stands for hydrogen, fluorine, chlorine or methyl,
R²⁰ stands for hydrogen, fluorine, chlorine or methyl,
and
R²⁵ stands for hydrogen, fluorine, chlorine or methyl,
   and their salts, solvates and solvates of the salts.

Within the scope of the present invention, compounds of formula (I) are especially preferred in which
Q stands for a group of formula

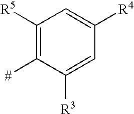

where
# denotes the site of attachment to the amino group,
R³ stands for hydroxycarbonyl,
R⁴ stands for hydrogen, fluorine, chlorine, methyl, ethyl, difluoromethyl, trifluoromethyl, methoxy, difluoromethoxy or trifluoromethoxy,
R⁵ stands for hydrogen or fluorine,
R¹ stands for hydrogen or methyl,
R² stands for phenyl,
   where phenyl can be substituted with 1 or 2 substituents selected independently of one another from the group comprising fluorine, chlorine, methyl, ethyl, trifluoromethyl, methoxy, ethoxy or trifluoromethoxy,
R⁶ stands for a group of formula

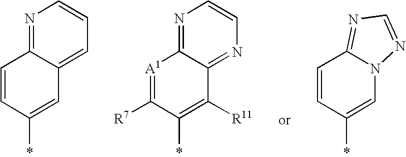

where
* denotes the site of attachment to the pyrazole,
A¹ stands for CR¹⁰ or N,
   in which
   R¹⁰ stands for hydrogen, fluorine, chlorine or methyl,
R⁷ stands for hydrogen, fluorine, chlorine or methyl,
R¹¹ stands for hydrogen, fluorine, chlorine or methyl,
   and their salts, solvates and solvates of the salts.

Within the scope of the present invention, compounds of formula (I) are also especially preferred in which
Q stands for a group of formula

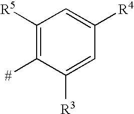

where
# denotes the site of attachment to the amino group,
R³ stands for hydroxycarbonyl,
R⁴ stands for hydrogen, fluorine, chlorine, methyl, ethyl, difluoromethyl, trifluoromethyl, methoxy, difluoromethoxy or trifluoromethoxy,
R⁵ stands for hydrogen,
R¹ stands for methyl,
R² stands for a group of formula

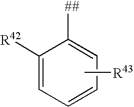

in which
## stands for the site of attachment to the pyrazole,
R⁴² stands for hydrogen, fluorine, chlorine, trifluoromethyl, methyl, ethyl, methoxy or ethoxy,
R⁴³ stands for hydrogen, fluorine, chlorine or methyl,
R⁶ stands for a group of formula

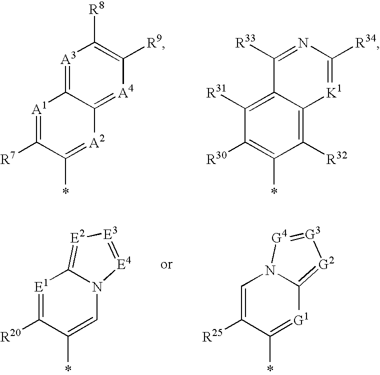

where
* denotes the site of attachment to the pyrazole,
A¹ stands for CR¹⁰ or N,
   in which
   R¹⁰ stands for hydrogen, fluorine or chlorine,
A² stands for CR^{H},
   in which
   R¹¹ stands for hydrogen, fluorine, chlorine or methyl,
A³ stands for N,
A⁴ stands for N,
E¹ stands for CR²¹,
   in which
   R²¹ stands for hydrogen,
E² stands for N,
E³ stands for CR²³,
   in which
   R²³ stands for hydrogen or amino,
E⁴ stands for N,
G¹ stands for CR²⁶,
   in which
   R²⁶ stands for hydrogen,
G² stands for N,
G³ stands for CR²⁸,
   in which
   R²⁸ stands for hydrogen or amino,
G⁴ stands for N,
K¹ stands for N,
R⁷ stands for hydrogen, fluorine, chlorine, methyl, methoxy or ethoxy,
R⁸ stands for hydrogen,
R⁹ stands for hydrogen,
R²⁰ stands for hydrogen, fluorine, chlorine, methyl, methoxy or ethoxy,
R²⁵ stands for hydrogen, fluorine, chlorine, methyl, methoxy or ethoxy,
R³⁰ stands for hydrogen, fluorine, chlorine, methyl, methoxy or ethoxy,
R³¹ stands for hydrogen, fluorine or chlorine,
R³² stands for hydrogen, fluorine or chlorine,
R³³ stands for hydrogen,
and
R³⁴ stands for hydrogen,
   and their salts, solvates and solvates of the salts.

Within the scope of the present invention, compounds of formula (I) are also preferred in which R³ stands for hydroxycarbonyl.

Within the scope of the present invention, compounds of formula (I) are also preferred in which R¹ stands for hydrogen.

Within the scope of the present invention, compounds of formula (I) are also preferred in which R¹ stands for methyl.

Within the scope of the present invention, compounds of formula (I) are also preferred in which
R² stands for phenyl,
   where phenyl can be substituted with 1 or 2 substituents selected independently of one another from the group comprising fluorine, chlorine, methyl, ethyl, trifluoromethyl, methoxy, ethoxy or trifluoromethoxy.

Within the scope of the present invention, compounds of formula (I) are also preferred in which
R² stands for a group of formula

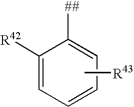

in which
## stands for the site of attachment to the pyrazole,
R⁴² stands for fluorine, chlorine, trifluoromethyl, methyl, ethyl, methoxy or ethoxy,
R⁴³ stands for hydrogen, fluorine, chlorine or methyl.

Within the scope of the present invention, compounds of formula (I) are also preferred in which
Q stands for a group of formula

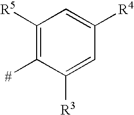

where
# denotes the site of attachment to the amino group,
R³ stands for hydroxycarbonyl,
R⁴ stands for hydrogen, fluorine, chlorine, methyl, ethyl, difluoromethyl, trifluoromethyl, methoxy, difluoromethoxy or trifluoromethoxy,
R⁵ stands for hydrogen or fluorine.

Within the scope of the present invention, compounds of formula (I) are also preferred in which
R⁶ stands for a group of formula

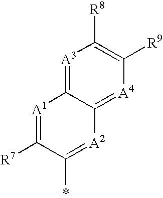

where
* denotes the site of attachment to the pyrazole,
A¹ stands for CR¹⁰,
   in which
   R¹⁰ stands for hydrogen, fluorine, chlorine or methyl,
A² stands for CR^{H},
   in which
   R¹¹ stands for hydrogen, fluorine, chlorine or methyl,
A³ stands for N,
A⁴ stands for N,
R⁷ stands for hydrogen, fluorine, chlorine, methyl, methoxy or ethoxy,
R⁸ stands for hydrogen, fluorine, chlorine, methyl or methoxy,
R⁹ stands for hydrogen, fluorine, chlorine, methyl, methoxy, amino, methylamino or dimethylamino.

Within the scope of the present invention, compounds of formula (I) are also preferred in which
R⁶ stands for a group of formula

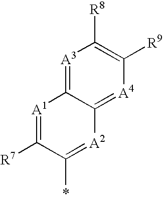

where
* denotes the site of attachment to the pyrazole,
A¹ stands for CR¹⁰,
   in which
   R¹⁰ stands for hydrogen or fluorine,
A² stands for CR^{H},
   in which
   R¹¹ stands for hydrogen,
A³ stands for N,
A⁴ stands for N,
R⁷ stands for hydrogen, fluorine, chlorine, methyl, methoxy or ethoxy,
R⁸ stands for hydrogen,
R⁹ stands for hydrogen.

Within the scope of the present invention, compounds of formula (I) are also preferred in which
R⁶ stands for a group of formula

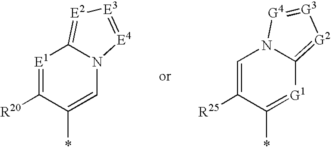

where
* denotes the site of attachment to the pyrazole,
E¹ stands for CR²¹,
   in which
   R²¹ stands for hydrogen or fluorine,
E² stands for N,
E³ stands for CR²³,
   in which
   R²³ stands for hydrogen or amino,
E⁴ stands for N,
G¹ stands for CR²⁶,
   in which
   R²⁶ stands for hydrogen or fluorine,
G² stands for N,
G³ stands for CR²⁸,
   in which
   R²⁸ stands for hydrogen or amino,
G⁴ stands for N,
R²⁰ stands for hydrogen, fluorine, chlorine, methyl, methoxy or ethoxy,
R²⁵ stands for hydrogen, fluorine, chlorine, methyl, methoxy or ethoxy.

The definitions of the residues stated individually in the respective combinations or preferred combinations of residues are also replaced at will with definitions of the residues of other combinations regardless of the respective combinations of residues stated.

Combinations of two or more of the aforementioned preferred ranges are quite especially preferred.

Another object of the invention is a method of production of the compounds according to the invention of formula (I-1), in which R³ stands for hydroxycarbonyl, characterized in that
[A] a compound of formula (II)

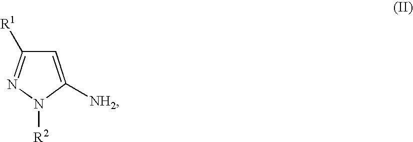

in which R¹ and R² have the respective meanings given above,
is transformed in an inert solvent with a halogenating agent to a compound of formula (III-A)

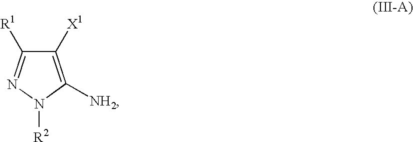

in which R¹ and R² have the respective meanings given above
and
X¹ stands for halogen, in particular for bromine or iodine,
this is then reacted in an inert solvent in the presence of a base and a suitable palladium catalyst with a compound of formula (IV)

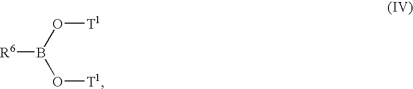

in which R⁶ has the meaning given above
and
T¹ stands for hydrogen or both residues T¹ together form a —C(CH₃)₂—C(CH₃)₂— or —CH₂C(CH₃)₂CH₂— bridge,
to a compound of formula (V-A)

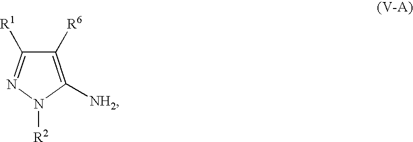

in which R¹, R² and R⁶ have the respective meanings given above,
and this is then reacted in an inert solvent in the presence of a suitable catalyst with a compound of formula (VI-A)

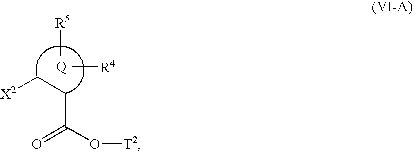

in which Q, R⁴ and R⁵ have the respective meanings given above
and
T² stands for (C₁-C₄)-alkyl,
X² stands for halogen, preferably bromine,
to a compound of formula (VII)

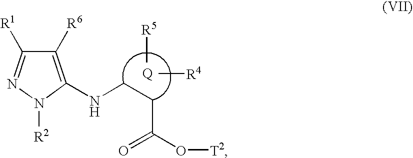

in which Q, T², R', R², R⁴, R⁵ and R⁶ have the respective meanings given above,
   or
[B] a compound of formula (II) is reacted in an inert solvent in the presence of a suitable catalyst with a compound of formula (VI-A) to a compound of formula (III-B)

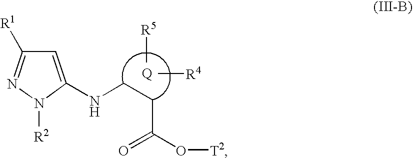

in which Q, R¹, R², R⁴ and R⁵ have the respective meanings given above, and
T² stands for (C₁-C₄)-alkyl,
and this is then transformed in an inert solvent with a halogenating agent to a compound of formula (V-B)

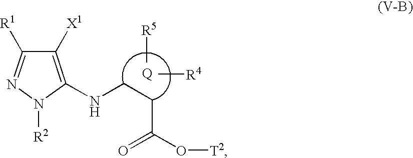

in which Q, T², X¹, R¹, R², R⁴ and R⁵ have the respective meanings given above,
and
X¹ stands for halogen, preferably bromine,
and this is then reacted in an inert solvent in the presence of a base and a suitable palladium catalyst with a compound of formula (IV) to a compound of formula (VII),
   or
[C] a compound of formula (VIII)

R⁶—X³ (VIII),
in which R⁶ has the meaning given above and
X³ stands for halogen, preferably bromine or iodine,
is reacted in an inert solvent in the presence of a suitable palladium catalyst with trimethylsilylacetonitrile to a compound of formula (IX)

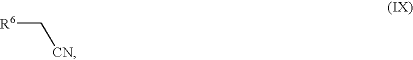

in which R⁶ has the meaning given above,
and this is then reacted in an inert solvent in the presence of a suitable base with an ester of formula (X)

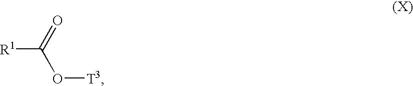

in which R¹ has the meaning given above and
T³ stands for (C₁-C₄)-alkyl,
to a compound of formula (XI)

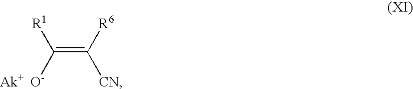

in which R¹ and R⁶ have the respective meanings given above and
Ak⁺ stands for an alkali ion, preferably sodium,
and this is then transformed with a hydrazine of formula (XII)

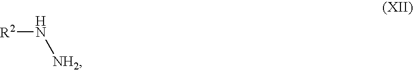

in which R² has the meaning given above,
to a compound of formula (V-A), and this is reacted further according to method [A] described above to a compound of formula (VII),
   and the compound of formula (VII) that results in each case is then transformed by hydrolysis of the ester to a carboxylic acid of formula (I-1)

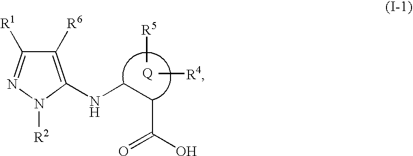

in which Q, R¹, R², R⁴, R⁵ and R⁶ have the respective meanings given above, and this is optionally reacted with the corresponding (i) solvents and/or (ii) bases or acids to its solvates, salts and/or solvates of the salts.

Elemental bromine with acetic acid, 1,3-dibromo-5,5-dimethylhydantoin and in particular N-bromosuccinimide (NBS), N-iodosuccinimide (NIS), optionally with addition of α,α′-azobis(isobutyronitrile) (AIBN) as initiator, are suitable as halogenating agents in steps (II)→(III-A) or (III-B)→(V-B).

The halogenation in steps (II)→(III-A) or (III-B)→(V-B) is carried out, when using NBS or NIS, preferably in acetonitrile in a temperature range from 0° C. to +100° C., and when using 1,3-dibromo-5,5-dimethylhydantoin preferably in dichloromethane in a temperature range from −20° C. to +30° C.

Inert solvents for steps (III-A)+(IV)→(V-A) and (V-B)+(IV)→(VII) are for example alcohols such as methanol, ethanol, n-propanol, isopropanol, n-butanol or tert.-butanol, ethers such as diethyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as dimethylformamide (DMF), dimethylsulfoxide (DMSO), N,N′-dimethylpropylene urea (DMPU), N-methylpyrrolidone (NMP), pyridine, acetonitrile or also water. It is also possible to use mixtures of the aforesaid solvents. A mixture of dimethylformamide and water is preferred.

Usual inorganic bases are suitable as bases for steps (III-A)+(IV)→(V-A) and (V-B)+(IV)→(VII). These include in particular alkali hydroxides, for example lithium, sodium or potassium hydroxide, alkali hydrogen carbonates such as sodium or potassium hydrogen carbonate, alkali or alkaline-earth carbonates such as lithium, sodium, potassium, calcium or cesium carbonate, or alkali hydrogen phosphates such as disodium or dipotassium hydrogen phosphate. Sodium or potassium carbonate is preferably used.

Palladium on activated charcoal, palladium(II) acetate, tetrakis-(triphenylphosphine)-palladium(0), bis-(triphenylphosphine)-palladium(II) chloride, bis-(acetonitrile)-palladium(II) chloride and [1,1′-bis(diphenylphosphino)ferrocene]dichloropalladium(II)-dichloromethane complex, for example, are suitable as palladium catalyst for steps (III-A)+(IV)→(V-A) and (V-B)+(IV)→(VII) [“Suzuki Coupling”] [cf. e.g. Hassan J. et al., Chem. Rev. 102, 1359-1469 (2002)].

Reactions (III-A)+(IV)→(V-A) and (V-B)+(IV)→(VII) are generally carried out in a temperature range from +20° C. to +150° C., preferably at +50° C. to +100° C.

Inert solvents for steps (V-A)+(VI-A)→(VII) and (II)+(VI-A)→(III-B) are for example ethers such as diethyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as dimethylformamide, dimethylsulfoxide, N,N′-dimethylpropylene urea (DMPU), N-methylpyrrolidone (NMP), pyridine, acetonitrile or also water. It is also possible to use mixtures of the aforesaid solvents. Preferably toluene is used.

The following are suitable as transition metal catalysts for the coupling reactions (V-A)+(VI-A)→(VII) and (II)+(VI-A)→(III-B): copper catalysts such as copper(I) iodide, and palladium catalysts such as palladium on activated charcoal, bis(dibenzylidene-acetone)-palladium(0), tris (dibenzylidene-acetone)-dipalladium(0), tetrakis(triphenylphosphine)-palladium(0), palladium(II) acetate, bis(triphenylphosphine)-palladium(II) chloride, bis(acetonitrile)-palladium(II) chloride or [1,1′-bis(diphenylphosphino)ferrocene]-palladium(II) chloride, optionally in conjunction with additional phosphane ligands, for example (2-biphenyl)di-tert.-butylphosphine, dicyclohexyl[2′,4′,6′-tris (1-methylethyl)biphenyl-2-yl]phosphane (XPHOS), bis(2-phenylphosphinophenyl)ether (DPEphos) or 4,5-bis(diphenylphosphino)-9,9-dimethylxanthene (Xantphos) [see also, e.g., Hassan J. et al., Chem. Rev. 102, 1359-1469 (2002); Farina V., Krishnamurthy V. and Scott W. J., in: The Stille Reaction, Wiley, New York, 1998].

Steps (V-A)+(VI-A)→(VII) and (II)+(VI-A)→(III-B) are generally carried out in a temperature range from +20° C. to +200° C., preferably from +80° C. to +180° C., optionally in a microwave. The reaction can take place at normal, increased or reduced pressure (e.g. from 0.5 to 5 bar). Generally it is carried out at normal pressure.

Step (VIII)→(IX) is carried out under the conditions described in Hartwig J. F. et al., J. Am. Chem. Soc. 2005, 127, 15824-15832.

In the reaction (IX)+(X)→(XI), inert solvents are for example ethers such as diethyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as dimethylformamide or acetonitrile. It is also possible to use mixtures of the aforesaid solvents. Preferably tetrahydrofuran is used.

The usual inorganic or organic bases are suitable as bases for this reaction. These include preferably alkali hydrides such as sodium hydride, alkali hydroxides for example lithium, sodium or potassium hydroxide, alkali alcoholates such as sodium or potassium methanolate, sodium or potassium ethanolate or potassium tert.-butylate, amides such as sodium amide, lithium, sodium or potassium bis-(trimethylsilyl)amide or lithium diisopropylamide or organometallic compounds such as butyllithium or phenyllithium. Sodium hydride is preferably used.

Step (IX)+(X)→(XI) is generally carried out in a temperature range from −78° C. to +100° C., preferably from −20° C. to +80° C., optionally in a microwave. The reaction can take place at normal, increased or reduced pressure (e.g. from 0.5 to 5 bar). Generally it is carried out at normal pressure.

Reaction (XI)+(XII)→(V-A) takes place for example under the conditions stated in Sorokin V. I. et al., Chem. Heterocycl. Comp. 2003, 39, 937-942.

Hydrolysis of the esters of compounds (VII) to compounds of formula (I-1) is carried out by usual methods, by treating the esters with acids or bases in inert solvents, in the latter case with the salts forming initially being transformed by treatment with acid to the free carboxylic acids. In the case of the tert.-butyl esters, cleavage of the esters is preferably carried out with acids.

Water or the usual organic solvents for ester cleavage are suitable as inert solvents for these reactions. These preferably include alcohols such as methanol, ethanol, n-propanol, isopropanol, n-butanol or tert.-butanol, or ethers such as diethyl ether, tetrahydrofuran, dioxane or glycol dimethyl ether, or other solvents such as acetone, dichloromethane, dimethylformamide or dimethylsulfoxide. It is also possible to use mixtures of the aforesaid solvents. In the case of basic ester hydrolysis, mixtures of water with dioxane, tetrahydrofuran, methanol and/or ethanol are preferably used, and in nitrile hydrolysis, preferably water and/or n-propanol. In the case of reaction with trifluoroacetic acid, dichloromethane is preferably used, and in the case of reaction with hydrogen chloride, preferably tetrahydrofuran, diethyl ether, dioxane or water is used.

The usual inorganic bases are suitable as bases. These preferably include alkali or alkaline-earth hydroxides, for example sodium, lithium, potassium or barium hydroxide, or alkali or alkaline-earth carbonates such as sodium, potassium or calcium carbonate. Sodium or lithium hydroxide is especially preferred.

Sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid or mixtures thereof optionally with addition of water are generally suitable as acids for ester cleavage. Hydrogen chloride or trifluoroacetic acid in the case of the tert.-butyl esters and hydrochloric acid in the case of the methyl esters are preferred.

Ester cleavage generally takes place in a temperature range from 0° C. to +100° C., preferably at +0° C. to +50° C.

The aforesaid reactions can be carried out at normal, increased or reduced pressure (e.g. from 0.5 to 5 bar). Generally the reaction is carried out at normal pressure in each case.

The compounds of formula (II) are commercially available, known from the literature or can be prepared by analogy with methods known in the literature [cf. e.g. WO 2004/050651 p. 18-19; Sorokin V. I. et al., Chem. Heterocycl. Comp. 2003, 39, 937-942].

The compounds of formulas (IV) and (VI-A) are commercially available, known from the literature or obtainable by commonly known methods.

The methods described previously for production of the compounds according to the invention can be illustrated by the following synthesis schemes:

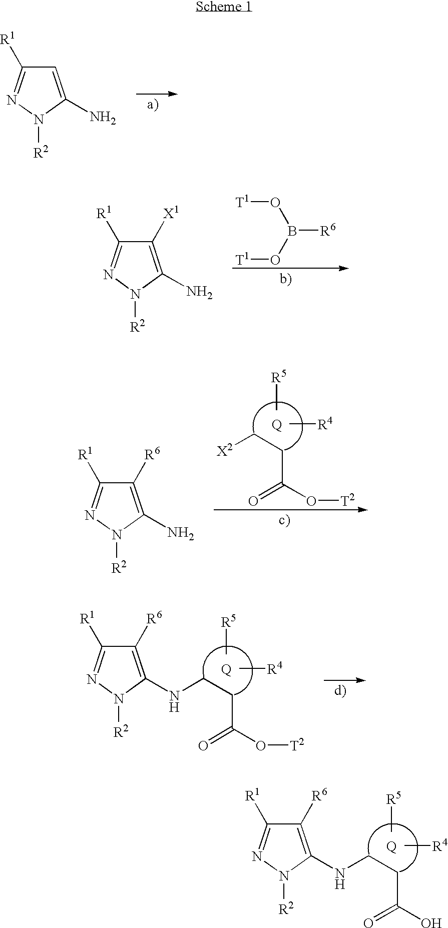

[a) for X¹=bromine, AcOH or N-bromosuccinimide, acetonitrile, RT to reflux temperature; for X¹═I: N-iodosuccinimide, acetonitrile, RT to reflux temperature; b) Pd(PPh₃)₄, Na₂CO₃ (aq), DMF, 110° C.; c) Pd(OAc)₂, K₃PO₄, (2-biphenyl)di-tert-butylphosphine, toluene, reflux temperature; d) NaOH (aq), dioxane/water, RT to reflux temperature].

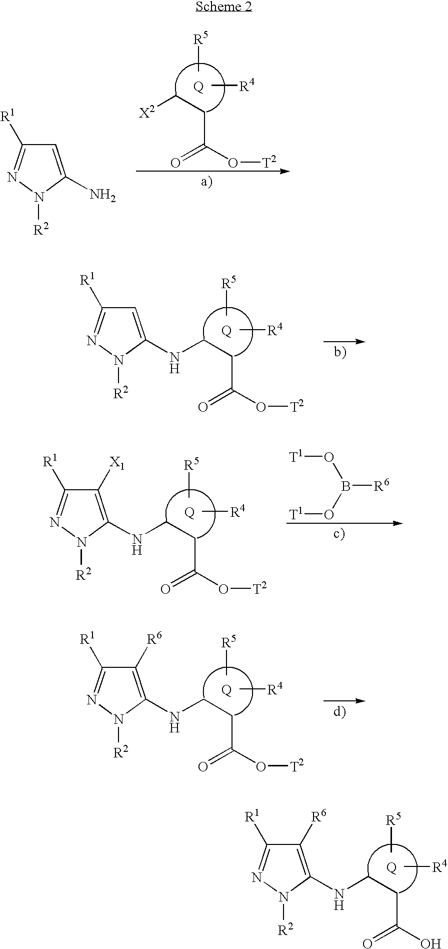

[a): Pd(OAc)₂, K₃PO₄, (2-biphenyl)di-tert-butylphosphine, toluene, reflux temperature; b): for X¹=bromine, AcOH or N-bromosuccinimide, acetonitrile, RT to reflux temperature; for X¹=I: N-iodosuccinimide, acetonitrile, RT to reflux temperature; c) Pd(PPh₃)₄, Na₂CO₃ (aq), DMF, 110° C.; d) NaOH (aq), dioxane/water, RT to reflux temperature].

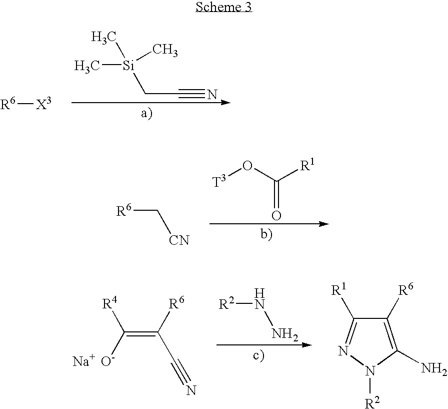

[a) tris(dibenzylidene-acetone)dipalladium, Xantphos, zinc fluoride, DMF, 90° C. [for X³═Br, I: see: Lingyun Wu, John F. Hartwig, J. Am. Chem. Soc. (2005), 127, 15824-15832.]; b) sodium hydride, THF, 0° C.->RT, then addition of the corresponding ester; RT->60° C.; c) 1N hydrochloric acid, reflux temperature].

Other compounds according to the invention of formula (I), in which R³ stands for aminocarbonyl, cyanoaminocarbonyl or (C₁-C₄)-alkylsulfonylaminocarbonyl, can be prepared by reacting the carboxylic acids according to the invention of formula (I-1) by the methods known by a person skilled in the art, cf. e.g. Kwon C.-H., Synth. Commun. 1987, 17, 1677-1682; McDonald I. M., J. Med. Chem. 2007, 50, 3101-3112.

The compounds according to the invention of formula (I), in which R³ stands for 1,3,4-oxadiazol-2(3H)on-5-yl, can be prepared by transforming a compound of formula (VII) first in an inert solvent with hydrazine to a compound of formula (XIII)

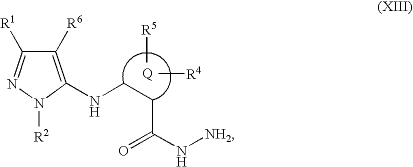

in which Q, R', R², R⁴, R⁵ and R⁶ have the meanings given above,
and then reacting in an inert solvent with phosgene or a phosgene equivalent, for example N,N′-carbonyldiimidazole.

In particular, alcohols such as methanol, ethanol, n-propanol, isopropanol, n-butanol or tert.-butanol, or ethers such as diethyl ether, dioxane, tetrahydrofuran, glycol dimethyl ether or diethylene glycol dimethyl ether, are suitable as inert solvents for the first step of this sequence of reactions. It is also possible to use mixtures of these solvents. A mixture of methanol and tetrahydrofuran is preferably used. The second reaction step is preferably carried out in an ether, in particular in tetrahydrofuran. The reactions generally take place in a temperature range from 0° C. to +70° C. under normal pressure.

The compounds according to the invention of formula (I), in which R³ stands for 1,2,4-oxadiazol-5(2H)on-3-yl, can be prepared by transforming a compound of formula (VI-B)

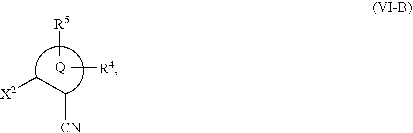

in which Q, X², R⁴ and R⁵ have the respective meanings given above,
by methods known from the literature to a compound of formula (XIV)

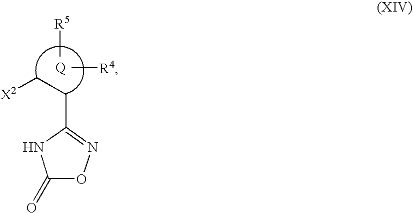

in which Q, X², R⁴ and R⁵ have the respective meanings given above,
this is then provided with a suitable protective group PG¹ and the resultant compound of formula (XV)

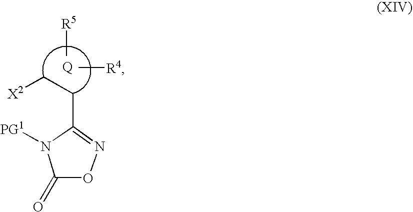

in which Q, X², R⁴ and R⁵ have the respective meanings given above
and
PG¹ stands for a protective group,
is reacted further according to methods [A] or [B] described above to a compounds of formula (XV)

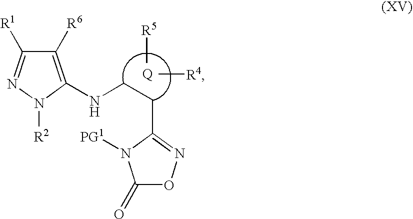

in which Q, R¹, R², R⁴, R⁵, R⁶ and PG¹ have the respective meanings given above,
then the protective group is cleaved by standard methods and the resultant compound of formula (I-2)

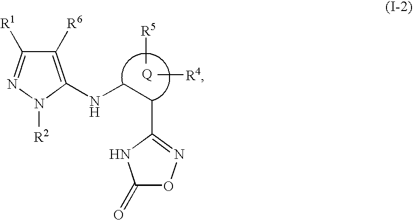

in which Q, R', R², R⁴, R⁵ and R⁶ have the respective meanings given above,
is reacted optionally with the corresponding (i) solvents and/or (ii) bases or acids to its solvates, salts and/or solvates of the salts.

Reaction (VI-B)→(XIV) is carried out by methods known by a person skilled in the art [cf. e.g. Iwao M., Kurashi T., J. Heterocycl. Chem. 1979, 16, 689-698].

For example allyl, trityl, 2-nitrobenzyl, 2-trimethylsilylethoxymethyl (SEM), 2-cyanoethyl, methoxybenzyl, dimethoxybenzyl and trimethoxybenzyl are suitable as protective groups PG¹ in the reaction (XIV)→(XV) [cf. e.g. Weller H. N. et al., Heterocycles 1993, 36, 1027-1038]. Cleavage of the protective groups in the reaction (XV)→(1-2) is carried out by methods known by a person skilled in the art [cf. Green T. W., Wuts P. G. M., Protective Groups in Organic Synthesis, 3rd Ed., John Wiley and Sons, 1999].

The compounds according to the invention of formula (I), in which R³ stands for tetrazol-5-yl, can be prepared by transforming a compound of formula (VI-B) by methods known from the literature to a compound of formula (XVI)

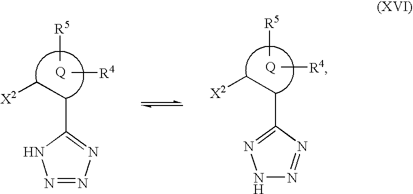

in which Q, X², R⁴ and R⁵ have the respective meanings given above,
this is then provided with a suitable protective group PG² and the resultant compound of formula (XVII-A) or (XVII-B)

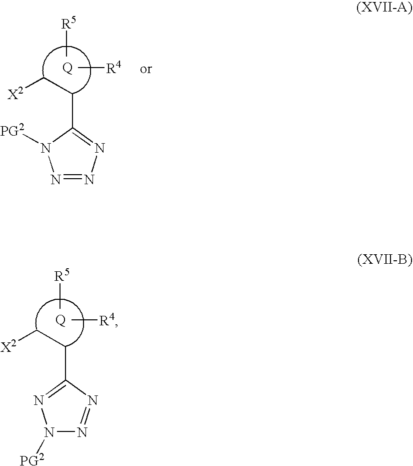

in which Q, X², R⁴ and R⁵ have the respective meanings given above
and
PG² stands for a protective group,
is reacted further according to methods [A] or [B] described above to a compounds of formula (XVIII-A) or (XVIII-B)

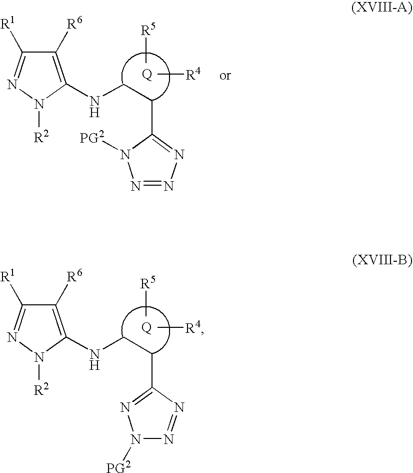

in which Q, R', R², R⁴, R⁵, R⁶ and PG² have the respective meanings given above,
then the protective group is cleaved by standard methods and the resultant compound of formula (I-3)

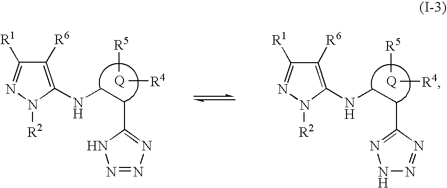

in which Q, R', R², R⁴, R⁵ and R⁶ have the respective meanings given above,
is reacted optionally with the corresponding (i) solvents and/or (ii) bases or acids to its solvates, salts and/or solvates of the salts.

Reaction (VI-B)→(XVI) is carried out by methods known by a person skilled in the art [cf. e.g. Kivrakidou O., Bräse S., Hülshorst F., Griebenow N., Org. Lett. 2004, 6, 1143; Wittenberger S. J., Donner B. G., J. Org. Chem. 1993, 58, 4139.].

For example allyl, trityl, 2-nitrobenzyl, 2-trimethylsilylethoxymethyl (SEM), 2-cyanoethyl, methoxybenzyl, dimethoxybenzyl and trimethoxybenzyl are suitable as protective groups PG² in reaction (XVI)→(XVII-A) or (XVII-B) [cf. e.g. Kerdesky F. A. J., Synth. Commun. 1996, 26, 1007-1013]. Cleavage of the protective groups in the reaction (XVIII-A) or (XVIII-B)→(1-3) is carried out by methods known by a person skilled in the art [cf. Green T. W., Wuts P. G. M., Protective Groups in Organic Synthesis, 3rd Ed., John Wiley and Sons, 1999].

The compounds of formula (VI-B) are commercially available, known from the literature or are obtainable by commonly known methods.

The compounds according to the invention possess valuable pharmacological properties and can be used for the prevention and/or treatment of various diseases and pathological states in humans and animals.

The compounds according to the invention are potent, selective adenosine A1 receptor antagonists, which inhibit adenosine activity in vitro and in vivo.

“Selective ligands to the adenosine A1 receptor” denotes, within the scope of the present invention, those adenosine receptor ligands for which on the one hand a definite action can be observed on the A1 adenosine receptor and on the other hand no action, or a definite weaker action (factor of 10 or more) can be observed on A2a, A2b and A3 adenosine receptor subtypes, and regarding the test methods for the selectivity of action, reference is made to the tests described in section B-1.

The compounds according to the invention are suitable in particular for the prophylaxis and/or treatment of cardiovascular diseases. In this connection, the following may be mentioned for example and preferably as target indications: acute and chronic heart failure, acute decompensated heart failure, arterial hypertension, coronary heart disease, stable and unstable angina pectoris, myocardial ischemia, myocardial infarction, shock, arteriosclerosis, atrial and ventricular arrhythmias, transient and ischemic attacks, stroke, inflammatory cardiovascular diseases, peripheral and cardiac vessel diseases, peripheral impaired perfusion, arterial pulmonary hypertension, spasms of the coronary arteries and peripheral arteries, thromboses, thromboembolic diseases, development of edema such as pulmonary edema, cerebral edema, renal edema or edema due to heart failure, and restenoses such as after thrombolytic therapies, percutaneous-transluminal angioplasty (PTA), transluminal coronary angioplasty (PTCA), heart transplant and bypass surgery.

In the sense of the present invention, the term heart failure also includes more specific or related pathologies such as right heart failure, left heart failure, total heart failure, ischemic cardiomyopathy, congestive cardiomyopathy, congenital heart defects, valvular defects, heart failure with valvular defects, mitral valve stenosis, mitral insufficiency, aortic valve stenosis, aortic valve insufficiency, tricuspid stenosis, tricuspid insufficiency, pulmonary stenosis, pulmonary insufficiency, combined valvular defects, myocarditis, chronic myocarditis, acute myocarditis, viral myocarditis, diabetic heart failure, alcoholic cardiomyopathy, cardiac storage diseases, diastolic heart failure and systolic heart failure.

Furthermore, the compounds according to the invention are suitable for use as diuretics for the treatment of edemas and in electrolyte disturbances, in particular in hypervolemic and euvolemic hyponatremia.

The compounds according to the invention are also suitable for the prophylaxis and/or treatment of polycystic kidney disease (PCKD) and of syndrome of inappropriate secretion of antidiuretic hormone (SIADH).

Furthermore, the compounds according to the invention are suitable for the treatment and/or prophylaxis of kidney diseases, in particular of renal insufficiency, and of acute and chronic renal failure. In the sense of the present invention, the term renal insufficiency comprises both acute and chronic forms of renal insufficiency, as well as underlying or related kidney diseases such as renal hypoperfusion, intradialytic hypotension, obstructive uropathy, glomerulonephritis, acute glomerulonephritis, tubulointerstitial diseases, nephropathic diseases such as primary and congenital kidney disease, nephritis, nephropathy induced by toxic substances, contrast-induced nephropathy, diabetic nephropathy, pyelonephritis, renal cysts and nephrosclerosis, which can be characterized diagnostically for example by abnormally reduced creatinine and/or water excretion, abnormally raised blood concentrations of urea, nitrogen, potassium and/or creatinine, altered activity of renal enzymes, e.g. glutamylsynthetase, altered urine osmolarity or urine volume, increased microalbuminuria, macroalbuminuria, lesions on glomeruli and arterioles, tubular dilatation, hyperphosphatemia and/or need for dialysis. The present invention also comprises the use of the compounds according to the invention for the treatment and/or prophylaxis of sequelae of renal insufficiency, for example pulmonary edema, heart failure, uraemia, anemia, electrolyte disturbances (e.g. hyperkalemia, hyponatremia) and disturbances in bone and carbohydrate metabolism.

Moreover, the compounds according to the invention can be used for the prophylaxis and/or treatment of hepatic cirrhosis, ascites, diabetes mellitus and diabetic sequelae, e.g. neuropathy.

In addition, the compounds according to the invention are suitable for the prophylaxis and/or treatment of central nervous system disturbances such as anxiety states and depressions, glaucoma and cancer, in particular lung tumors.

Furthermore, the compounds according to the invention can be used for the prophylaxis and/or treatment of inflammatory diseases, asthmatic diseases, chronic-obstructive pulmonary diseases (COPD), pain states, prostatic hypertrophy, incontinence, cystitis, hyperactive bladder, diseases of the adrenal gland such as pheochromocytoma and adrenal apoplexy, diseases of the intestine, for example Crohn's disease and diarrhea, or disturbances of menstruation, for example dysmenorrhea.

A further object of the present invention is the use of the compounds according to the invention for the treatment and/or prophylaxis of diseases, in particular the aforementioned diseases.

The present invention also relates to the compounds according to the invention for use in a method of treatment and/or prophylaxis of acute decompensated and chronic heart failure, hypervolemic and euvolemic hyponatremia, hepatic cirrhosis, ascites, edemas, nephropathy, acute and chronic renal failure, renal insufficiency and syndrome of inappropriate secretion of antidiuretic hormone (SIADH).

The present invention also relates to the use of the compounds according to the invention for the production of a medicinal product for the treatment and/or prophylaxis of diseases, in particular the aforementioned diseases.

The present invention also relates to a method of treatment and/or prophylaxis of diseases, in particular the aforementioned diseases, using an effective amount of at least one of the compounds according to the invention.

The compounds according to the invention can be used alone or if necessary in combination with other active substances. The present invention also relates to medicinal products that contain at least one of the compounds according to the invention and one or more additional active substances, in particular for the treatment and/or prophylaxis of the aforementioned diseases. The following may be mentioned, as examples and preferably, as combination active substances that are suitable for this:
organic nitrates and NO donors, for example sodium nitroprusside, nitroglycerin, isosorbide mononitrate, isosorbide dinitrate, molsidomine or SIN-1, and inhalational NO;
diuretics, in particular loop diuretics and thiazides and thiazidelike diuretics;
compounds with positive inotropic action, for example cardiac glycosides (digoxin), beta-adrenergic and dopaminergic agonists such as isoproterenol, epinephrine, norepinephrine, dopamine and dobutamine;
compounds that inhibit the degradation of cyclic guanosine monophosphate (cGMP) and/or cyclic adenosine monophosphate (cAMP), for example inhibitors of phosphodiesterases (PDE) 1, 2, 3, 4 and/or 5, in particular PDE 5 inhibitors such as sildenafil, vardenafil and tadalafil, and PDE 3 inhibitors such as aminone and milrinone;
natriuretic peptides, e.g. “atrial natriuretic peptide” (ANP, anaritide), “B-type natriuretic peptide” or “brain natriuretic peptide” (BNP, nesiritide), “C-type natriuretic peptide” (CNP) and urodilatin;
calcium sensitizers, for example and preferably levosimendan;
NO- and heme-independent activators of guanylate cyclase, such as in particular the compounds described in WO 01/19355, WO 01/19776, WO 01/19778, WO 01/19780, WO 02/070462 and WO 02/070510;
NO-independent, but heme-dependent stimulators of guanylate cyclase, such as in particular the compounds described in WO 00/06568, WO 00/06569, WO 02/42301 and WO 03/095451;
antagonists of vasopressin receptors, for example conivaptan, tolvaptan, RWJ-676070 or RWJ-351647;
inhibitors of human neutrophil elastase (HNE), for example sivelestat or DX-890 (Reltran);
signal transduction cascade inhibiting compounds, for example tyrosine kinase inhibitors, in particular sorafenib, imatinib, gefitinib and erlotinib;
compounds having an effect on energy metabolism, for example and preferably etomoxir, perhexyline, dichloroacetate, ranolazine or trimetazidine;
agents with antithrombotic action, for example and preferably from the group comprising thrombocyte aggregation inhibitors, anticoagulants or profibrinolytic substances;
active substances that lower the blood pressure, for example and preferably from the group comprising calcium antagonists, angiotensin AII antagonists, ACE inhibitors, vasopeptidase inhibitors, inhibitors of neutral endopeptidase, endothelin antagonists, renin inhibitors, alpha-receptor blockers, beta-receptor blockers, mineralocorticoid receptor antagonists and rho-kinase inhibitors; and/or
active substances that modify fat metabolism, for example and preferably from the group comprising thyroid receptor agonists, cholesterol synthesis inhibitors, for example and preferably HMG-CoA-reductase or squalene synthesis inhibitors, ACAT inhibitors, CETP inhibitors, MTP inhibitors, PPAR-alpha-, PPAR-gamma- and/or PPAR-delta agonists, cholesterol absorption inhibitors, lipase inhibitors, polymeric bile acid adsorbers, bile acid reabsorption inhibitors and lipoprotein(a) antagonists;
inhibitors of Na⁺/K⁺ ATPase, for example istaroxime;
activators of the myosin chain, for example CK-1827452;
agonists of the LGR7 receptor, for example relaxin;
agonists of the CFR-R2 receptor, for example urocortin;
inhibitors of human chymase, for example TPC-806.

In a preferred embodiment of the invention, the compounds according to the invention are administered in combination with a diuretic, for example and preferably furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

“Agents with antithrombotic action” are preferably understood to be compounds from the group comprising thrombocyte aggregation inhibitors, anticoagulants or profibrinolytic substances.

In a preferred embodiment of the invention, the compounds according to the invention are administered in combination with a thrombocyte aggregation inhibitor, for example and preferably aspirin, clopidogrel, ticlopidine or dipyridamole.

In a preferred embodiment of the invention, the compounds according to the invention are administered in combination with a thrombin inhibitor, for example and preferably ximelagatran, Melagatran, bivalirudin or clexane.

In a preferred embodiment of the invention, the compounds according to the invention are administered in combination with a GPIIb/IIIa antagonist, for example and preferably tirofiban or abciximab.

In a preferred embodiment of the invention, the compounds according to the invention are administered in combination with a factor Xa inhibitor, for example and preferably rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, for example and preferably coumarin.

“Agents for lowering blood pressure” are preferably understood to be compounds from the group comprising calcium antagonists, angiotensin AII antagonists, ACE inhibitors, vasopeptidase inhibitors, inhibitors of neutral endopeptidase, endothelin antagonists, renin inhibitors, alpha-receptor blockers, beta-receptor blockers, mineralocorticoid receptor antagonists, rho-kinase inhibitors, prostanoid IP receptor agonists and diuretics.

In a preferred embodiment of the invention, the compounds according to the invention are administered in combination with a calcium antagonist, for example and preferably nifedipine, amlodipine, verapamil or diltiazem.

In a preferred embodiment of the invention, the compounds according to the invention are administered in combination with an angiotensin AII antagonist, for example and preferably losartan, candesartan, valsartan, telmisartan or embusartan.

In a preferred embodiment of the invention, the compounds according to the invention are administered in combination with an ACE inhibitor, for example and preferably enalapril, captopril, lisinopril, ramipril, delapril, fosinopril, quinopril, perindopril or trandopril.

In a preferred embodiment of the invention, the compounds according to the invention are administered in combination with a vasopeptidase inhibitor or inhibitor of neutral endopeptidase (NEP), for example and preferably omapatrilat or AVE-7688.

In a preferred embodiment of the invention, the compounds according to the invention are administered in combination with an endothelin antagonist, for example and preferably bosentan, darusentan, ambrisentan or sitaxsentan.

In a preferred embodiment of the invention, the compounds according to the invention are administered in combination with a renin inhibitor, for example and preferably aliskiren, SPP-600 or SPP-800.

In a preferred embodiment of the invention, the compounds according to the invention are administered in combination with an alpha-1-receptor blocker, for example and preferably prazosin.

In a preferred embodiment of the invention, the compounds according to the invention are administered in combination with a beta-receptor blocker, for example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with a mineralocorticoid receptor antagonist, for example and preferably spironolactone, eplerenone, canrenone or potassium canrenoate.

In a preferred embodiment of the invention, the compounds according to the invention are administered in combination with a rho-kinase inhibitor, for example and preferably fasudil, Y-27632, SAR407899, SLx-2119, BF-66851, BF-66852, BF-66853, KI-23095 or BA-1049.

In a preferred embodiment of the invention, the compounds according to the invention are administered in combination with an agonist of the prostanoid IP receptor, for example and preferably iloprost, treprostinil, beraprost or NS-304.

“Agents modifying fat metabolism” are preferably understood to be compounds from the group comprising CETP inhibitors, thyroid receptor agonists, cholesterol synthesis inhibitors such as HMG-CoA-reductase or squalene synthesis inhibitors, ACAT inhibitors, MTP inhibitors, PPAR-alpha-, PPAR-gamma- and/or PPAR-delta agonists, cholesterol absorption inhibitors, polymeric bile acid adsorbers, bile acid reabsorption inhibitors, lipase inhibitors and lipoprotein(a) antagonists.

In a preferred embodiment of the invention, the compounds according to the invention are administered in combination with a CETP inhibitor, for example and preferably torcetrapib (CP-529 414), JJT-705, BAY 60-5521, BAY 78-7499 or CETP-vaccine (Avant).

In a preferred embodiment of the invention, the compounds according to the invention are administered in combination with a thyroid receptor agonist, for example and preferably D-thyroxine, 3,5,3′-triiodothyronine (T3), CGS 23425 or axitirome (CGS 26214).

In a preferred embodiment of the invention, the compounds according to the invention are administered in combination with an HMG-CoA-reductase inhibitor from the statins class, for example and preferably lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, for example and preferably BMS-188494 or TAK-475.

In a preferred embodiment of the invention, the compounds according to the invention are administered in combination with an ACAT inhibitor, for example and preferably avasimibe, melinamide, pactimibe, eflucimibe or SMP-797.

In a preferred embodiment of the invention, the compounds according to the invention are administered in combination with an MTP inhibitor, for example and preferably implitapide, BMS-201038, R-103757 or JTT-130.

In a preferred embodiment of the invention, the compounds according to the invention are administered in combination with a PPAR-gamma agonists, for example and preferably pioglitazone or rosiglitazone.

In a preferred embodiment of the invention, the compounds according to the invention are administered in combination with a PPAR-delta agonist, for example and preferably GW-501516 or BAY 68-5042.

In a preferred embodiment of the invention, the compounds according to the invention are administered in combination with a cholesterol absorption inhibitor, for example and preferably ezetimibe, tiqueside or pamaqueside.

In a preferred embodiment of the invention, the compounds according to the invention are administered in combination with a lipase inhibitor, for example and preferably orlistat.

In a preferred embodiment of the invention, the compounds according to the invention are administered in combination with a polymeric bile acid adsorber, for example and preferably cholestyramine, colestipol, colesolvam, CholestaGel or colestimide.

In a preferred embodiment of the invention, the compounds according to the invention are administered in combination with a bile acid reabsorption inhibitor, for example and preferably ASBT (=IBAT) inhibitors, for example AZD-7806, S-8921, AK-105, BARI-1741, SC-435 or SC-635.

In a preferred embodiment of the invention, the compounds according to the invention are administered in combination with a lipoprotein(a) antagonist, for example and preferably Gemcabene calcium (CI-1027) or nicotinic acid.

The present invention also relates to medicinal products that contain at least one compound according to the invention, usually together with one or more inert, nontoxic, pharmaceutically suitable excipients, and use thereof for the aforementioned purposes.

The compounds according to the invention can act systemically and/or locally. For this purpose, they can be applied by a suitable route, e.g. oral, parenteral, pulmonary, nasal, sublingual, lingual, buccal, rectal, dermal, transdermal, conjunctival, otic or as implant or stent.

For these routes of administration, the compounds according to the invention can be administered in suitable dosage forms.

Dosage forms that contain the compounds according to the invention in crystalline and/or amorphous and/or dissolved form, functioning according to the state of the art, and providing rapid or modified release of the compounds according to the invention, are suitable for oral administration, for example tablets (non-coated or coated tablets, for example with enteric coatings or slowly dissolving or insoluble coatings, which control the release of the compound according to the invention), tablets that disintegrate quickly in the oral cavity or films/wafers, films/lyophilizates, capsules (for example hard or soft gelatin capsules), sugar-coated tablets, granules, pellets, powders, emulsions, suspensions, aerosols or solutions.

Parenteral application can take place with avoidance of an absorption step (e.g. intravenous, intraarterial, intracardial, intraspinal or intralumbar) or with inclusion of absorption (e.g. intramuscular, subcutaneous, intracutaneous, percutaneous or intraperitoneal). Among others, injection and infusion preparations in the form of solutions, suspensions, emulsions, lyophilizates or sterile powders are suitable as dosage forms for parenteral application.

Inhalational dosage forms (among others, powder inhalers, nebulizers), nasal drops, solutions or sprays, tablets, films/wafers or capsules for lingual, sublingual or buccal application, suppositories, ear or eye preparations, vaginal capsules, aqueous suspensions (lotions, shaking mixtures), lipophilic suspensions, ointments, creams, transdermal therapeutic systems (e.g. patches), milks, pastes, foams, dusting powders, implants or stents, for example, are suitable for the other routes of administration.

Oral or parenteral application, in particular oral and intravenous application, are preferred.

The compounds according to the invention can be transformed to the stated dosage forms. This can be carried out in a manner known per se, by mixing with inert, nontoxic, pharmaceutically suitable excipients. These excipients include, among others, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and taste and/or odor correctants.

Generally it has proved advantageous, in parenteral application, to administer amounts from about 0.001 to 10 mg/kg, preferably about 0.01 to 1 mg/kg of body weight to achieve effective results. In oral application, the dosage is about 0.01 to 100 mg/kg, preferably about 0.01 to 20 mg/kg and quite especially preferably 0.1 to 10 mg/kg of body weight.

Nevertheless, it may possibly be necessary to deviate from the stated amounts, depending on body weight, route of administration, individual response to the active substance, type of preparation and time point or interval in which administration takes place. Thus, it may be sufficient in some cases to use less than the aforementioned minimum amount, whereas in other cases the stated upper limit has to be exceeded. If larger amounts are being administered, it may be advisable to divide these into several individual doses over the day.

The following examples of application explain the invention. The invention is not limited to the examples.

The percentages in the following tests and examples are, unless stated otherwise, percentages by weight; parts are parts by weight. Proportions of solvents, dilution ratios and concentrations of liquid/liquid solutions are always based on volume.

### A. EXAMPLES

### Abbreviations and Acronyms

Ac acetyl
AcOH acetic acid
aq. aqueous
TLC thin layer chromatography
DCI direct chemical ionization (in MS)
DMF dimethylformamide
DMSO dimethylsulfoxide
of theor. of theory (relating to yield)
eq. equivalent(s)
ESI electrospray ionization (in MS)
h hour(s)
Hal halogen
HPLC high-performance (high-pressure) liquid chromatography
LC-MS liquid chromatography coupled with mass spectrometry
min minute(s)
MPLC medium pressure liquid chromatography
MS mass spectrometry
mc multiplet, centered (in NMR)
NMR nuclear magnetic resonance spectrometry
Pd(PPh₃)₄ tetrakis(triphenylphosphine)palladium(0)
RP reverse phase (in HPLC)
RT room temperature
Rₜ retention time (in HPLC)
sbr singlet, broad (in NMR)
THF tetrahydrofuran
UV ultraviolet spectrometry
v/v volume-to-volume ratio (of a mixture)

### LC-MS, GC-MS and HPLC Methods:

### Method 1 (LC-MS):

Equipment type MS: Micromass ZQ; equipment type HPLC: HP 1100 Series; UV DAD; column: Phenomenex Gemini 3 u 30 mm×3.00 mm; eluent A: 1 l water+0.5 ml 50% formic acid, eluent B: 1 l acetonitrile+0.5 ml 50% formic acid; gradient: 0.0 min 90% A→2.5 mM 30% A→3.0 mM 5% A→4.5 min 5% A; flow: 0.0 min 1 ml/min→2.5 min/3.0 min/4.5 min 2 ml/min; furnace: 50° C.; UV detection: 210 nm.

### Method 2 (LC-MS):

Instrument: Micromass Quattro Micro MS with HPLC Agilent Series 1100; column: Thermo Hypersil GOLD 3μ 20×4 mm; eluent A: 1 l water+0.5 ml 50% formic acid, eluent B: 1 l acetonitrile+0.5 ml 50% formic acid; gradient: 0.0 min 100% A→3.0 min 10% A→4.0 min 10% A→4.01 min 100% A (flow 2.5 ml)→5.00 min 100% A furnace: 50° C.; flow: 2 ml/min; UV detection: 210 nm

### Method 3 (LC-MS):

Equipment type MS: Micromass ZQ; equipment type HPLC: Waters Alliance 2795; column: Phenomenex Synergi 2.5μ MAX-RP 100A Mercury 20 mm×4 mm; eluent A: 1 l water+0.5 ml 50% formic acid, eluent B: 1 l acetonitrile+0.5 ml 50% formic acid; gradient: 0.0 min 90% A→0.1 min 90% A→3.0 min 5% A→4.0 min 5% A→4.01 min 90% A; flow: 2 ml/min; furnace: 50° C.; UV detection: 210 nm.

### Method 4 (LC-MS):

Instrument: Micromass QuattroPremier with Waters HPLC Acquity; column: Thermo Hypersil GOLD 1.9μ 50×1 mm; eluent A: 1 l water+0.5 ml 50% formic acid, eluent B: 1 l acetonitrile+0.5 ml 50% formic acid; gradient: 0.0 min 90% A→0.1 min 90% A→1.5 min 10% A→2.2 min 10% A furnace: 50° C.; flow: 0.33 ml/min; UV detection: 210 nm

### Method 5 (LC-MS):

Instrument: Micromass Platform LCZ with HPLC Agilent Series 1100; column: Thermo Hypersil GOLD 3μ 20×4 mm; eluent A: 1 l water+0.5 ml 50% formic acid, eluent B: 1 l acetonitrile+0.5 ml 50% formic acid; gradient: 0.0 min 100% A→0.2 min 100% A→2.9 min 30% A→3.1 min 10% A→5.5 min 10% A; furnace: 50° C.; flow: 0.8 ml/min; UV detection: 210 nm

### Method 6 (GC-MS):

Instrument: Micromass GCT, GC6890; column: Restek RTX-35, 15 m×200 μm×0.33 μm; constant flow with helium: 0.88 ml/min; furnace: 70° C.; inlet: 250° C.; gradient: 70° C., 30° C./min→310° C. (3 min hold).

### Method 7 (Preparative HPLC):

Instrument: Abimed Gilson Pump 305/306, Manometric Module 806; column: Grom-Sil 1200DS-4HE 10 μm, 250 mm×40 mm; eluent: A=water, B=acetonitrile; gradient: 0.0 min 10% B→5 min 10% B→27 min 98% B→35 min 98% B→35.01 min 10% B→38 min 10% B; flow: 50 ml/min; column temperature: RT; UV detection: 210 nm.

### Method 8 (Preparative HPLC):

Instrument: Abimed Gilson Pump 305/306, Manometric Module 806; column: Grom-Sil 1200DS-4HE 10 μm, 250 mm×40 mm; eluent: A=water+0.075% formic acid, B=acetonitrile; gradient: 0.0 min 10% B→5 min 10% B→27 min 98% B→35 min 98% B→35.01 min 10% B→38 min 10% B; flow: 50 ml/min; column temperature: RT; UV detection: 210 nm.

### Method 9 (LC-MS):

Equipment type MS: Waters ZQ; equipment type HPLC: Agilent 1100 Series; UV DAD; column: Thermo Hypersil GOLD 3μ 20 mm×4 mm; eluent A: 1 l water+0.5 ml 50% formic acid, eluent B: 1 l acetonitrile+0.5 ml 50% formic acid; gradient: 0.0 min 100% A→3.0 min 10% A→4.0 min 10% A→4.1 min 100% flow: 2.5 ml/min, furnace: 55° C.; flow 2 ml/min; UV detection: 210 nm.

### Method 10 (LC-MS):

Instrument: Waters ACQUITY SQD HPLC System; column: Waters Acquity HPLC HSS T3 1.8μ 50×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; furnace: 50° C.; flow: 0.40 ml/min; UV detection: 210-400 nm.

### Starting Compounds and Intermediates:

### Example 1A

### 4-(Quinoxalin-6-yl)-3-methyl-1-(2-methylphenyl)-1H-pyrazol-5-amine

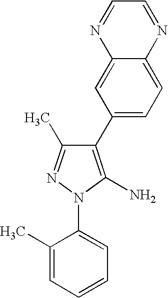

1.00 g (3.75 mmol) of 4-bromo-3-methyl-1-(2-methylphenyl)-1H-pyrazol-5-amine [Michaelis, Kappert, Justus Liebigs Ann. Chem. 1913, 397, 157] and 1.58 g (7.51 mmol) quinoxalin-6-yl boric acid hydrochloride were dissolved in 15 ml DMF. After adding 4 ml (8.00 mmol) of 2M aqueous sodium carbonate solution it was outgassed with argon. 261 mg (3.75 mmol) of tetrakis(triphenylphosphine)palladium(0) was added and a temperature of 110° C. was maintained for 4 h. After completion of reaction had been detected by TLC, 100 ml ethyl acetate was added and the solid residue was removed by filtration on kieselguhr. The resultant solution was washed with 100 ml water and with 100 ml of saturated aqueous sodium chloride solution. The organic phase was dried over sodium sulfate and the solvent was removed in a rotary evaporator. The raw product was purified by MPLC (Puriflash Analogix: 40M: isohexane/ethyl acetate=1/1). This gave 344 mg (29% of theor.) of the target compound.

LC-MS (method 1): Rₜ=1.69 min; MS (EIpos): m/z=316 [M+H]⁺.

¹H-NMR (400 MHz, DMSO-D₆): δ [ppm]=2.10 (s, 3H), 2.29 (s, 3H), 5.20 (s, 2H), 7.30-7.39 (m, 2H), 7.40-7.44 (m, 2H), 7.97 (dd, 1H), 8.00 (d, 1H), 8.09 (d, 1H). 8.86 (d, 1H), 8.91 (d, 1H).

### Example 2A

### Methyl-2-{[4-(quinoxalin-6-yl)-3-methyl-1-(2-methylphenyl)-1H-pyrazol-5-yl]amino}-5-fluorobenzene carboxylate

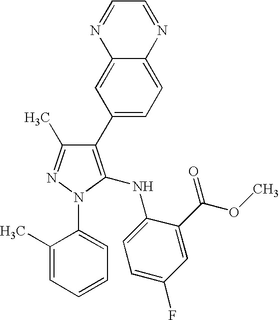

84.0 mg (0.266 mmol) of example 1A, 74.4 mg (0.320 mmol) of methyl-2-bromo-5-fluorobenzoate [Gerard, et al., Tetrahedron Lett. 2007, 48, 4123] and 79.2 mg (0.373 mmol) potassium phosphate were dissolved in 2.1 ml absolute toluene. The resultant solution was outgassed with argon. Then 5.4 mg (0.024 mmol) of palladium(II) acetate and 10.7 mg (0.036 mmol) of (2-biphenyl)di-tert.-butylphosphine were added. It was stirred for 72 h at the reflux temperature. After completion of reaction had been detected by TLC, 50 ml ethyl acetate was added and the solid residue was removed by filtration on kieselguhr. The resultant solution was neutralized with 1N hydrochloric acid and then washed with 50 ml water and with 50 ml of saturated aqueous sodium chloride solution. The organic phase was dried over sodium sulfate and the solvent was removed in a rotary evaporator. The raw product was purified by preparative HPLC (eluent: acetonitrile/water, gradient 10:90→90:10). This gave 10 mg (8% of theor.) of the target compound.

LC-MS (method 2): Rₜ=2.41 min; MS (EIpos): m/z=468 [M+H]⁺.

¹H-NMR (400 MHz, DMSO-D₆): δ [ppm]=2.17 (s, 3H), 3.81 (s, 3H), 6.50 (dd, 1H), 7.08 (mc, 1H), 7.25 (m, 1H). 7.30-7.34 (m, 2H), 7.37 (dd, 1H), 7.42 (d, 1H), 7.98 (dd, 1H), 8.05 (d, 1H), 8.11 (d, 1H), 8.89 (d, 1H), 8.91 (d, 1H), 9.07 (s, 1H).

### Example 3A

### Methyl-2-{[4-(quinoxalin-6-yl)-3-methyl-1-(2-methylphenyl)-1H-pyrazol-5-yl]amino}-5-chlorobenzene carboxylate

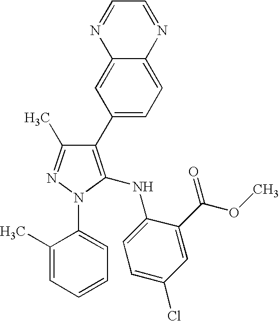

80.0 mg (0.254 mmol) of example 1A, 75.9 mg (0.304 mmol) of methyl-2-bromo-5-chlorobenzoate [Pan, Fletcher, J. Med. Chem. 1970, 13, 567] and 75.4 mg (0.355 mmol) potassium phosphate were dissolved in 2.0 ml absolute toluene. The resultant solution was outgassed with argon. Then 5.1 mg (0.023 mmol) of palladium(II) acetate and 10.2 mg (0.034 mmol) of (2-biphenyl)di-tert.-butylphosphine were added. It was stirred for 72 h at the reflux temperature. Then 50 ml ethyl acetate was added and the solid residue was removed by filtration on kieselguhr. The resultant filtrate was concentrated by evaporation and the raw product was purified by preparative HPLC (eluent: acetonitrile/water, gradient 10:90→90:10). This gave 59 mg (48% of theor.) of the target compound.

LC-MS (method 2): Rₜ=2.56 min; MS (EIpos): m/z=484 [M+H]⁺.

¹H-NMR (400 MHz, DMSO-D₆): δ [ppm]=2.19 (s, 3H), 2.49 (s, 3H), 3.81 (s, 3H), 6.50 (dd, 1H), 7.18-7.29 (m, 2H), 7.29-7.35 (m, 2H), 7.44 (d, 1H), 7.61 (d, 1H), 7.98 (dd, 1H), 8.06 (d, 1H), 8.13 (d, 1H), 8.89 (d, 1H), 8.91 (d, 1H), 9.21 (s, 1H).

### Example 4A

### Ethyl-2-{[4-(quinoxalin-6-yl)-3-methyl-1-(2-methylphenyl)-1H-pyrazol-5-yl]amino}-5-methyl-benzoate

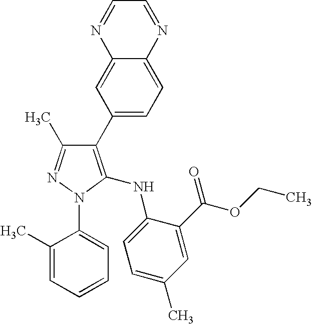

84.0 mg (0.266 mmol) of example 1A, 77.7 mg (0.320 mmol) of ethyl-2-bromo-5-methylbenzoate and 79.2 mg (0.373 mmol) potassium phosphate were dissolved in 2.0 ml absolute toluene. The resultant solution was outgassed with argon. Then 5.4 mg (0.024 mmol) of palladium(II) acetate and 10.7 mg (0.036 mmol) of (2-biphenyl)di-tert.-butylphosphine were added. It was stirred for 72 h at the reflux temperature. Then 50 ml ethyl acetate was added and the solid residue was removed by filtration on kieselguhr. The resultant filtrate was concentrated by evaporation and the raw product was purified by preparative HPLC (eluent: acetonitrile/water, gradient 10:90→90:10). This gave 18 mg (14% of theor.) of the target compound.

LC-MS (method 3): Rₜ=2.45 min; MS (EIpos): m/z=478 [M+H]⁺.

¹H-NMR (400 MHz, DMSO-D₆): δ [ppm]=1.27 (t, 3H), 2.04 (s, 3H), 2.17 (s, 3H), 2.48 (s, 3H), 4.24 (q, 2H), 6.40 (d, 1H), 6.98 (dd, 1H), 7.24 (m, 1H), 7.30-7.34 (m, 2H), 7.41 (d, 1H), 7.48 (d, 1H), 7.98 (dd, 1H), 8.04 (d, 1H), 8.11 (d, 1H), 8.88 (d, 1H), 8.90 (d, 1H), 9.10 (s, 1H).

### Example 5A

### Methyl-2-{[4-(quinoxalin-6-yl)-3-methyl-1-(2-methylphenyl)-1H-pyrazole-5-1]amino}benzoate

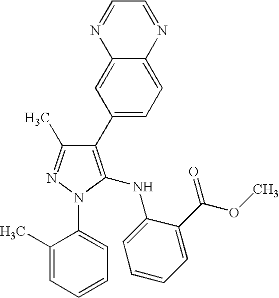

84.0 mg (0.266 mmol) of example 1A, 68.7 mg (0.320 mmol) of methyl-2-bromobenzoate and 79.2 mg (0.373 mmol) potassium phosphate were dissolved in 2.0 ml absolute toluene. The resultant solution was outgassed with argon. Then 5.4 mg (0.024 mmol) of palladium(II) acetate and 10.7 mg (0.036 mmol) of (2-biphenyl)di-tert.-butylphosphine were added. It was stirred for 48 h at the reflux temperature. Then 50 ml ethyl acetate was added and the solid residue was removed by filtration on kieselguhr. The resultant filtrate was concentrated by evaporation and the raw product was purified by preparative HPLC (eluent: acetonitrile/water, gradient 10:90→90:10). This gave 27 mg (23% of theor.) of the target compound.

LC-MS (method 3): Rₜ=2.18 min; MS (EIpos): m/z=450 [M+H]⁺.

¹H-NMR (400 MHz, DMSO-D₆): δ [ppm]=2.18 (s, 3H), 2.48 (s, 3H), 3.80 (s, 3H), 6.48 (dd, 1H), 6.59 (mc, 1H), 7.14 (mc, 1H), 7.24 (mc, 1H), 7.28-7-34 (m, 2H), 7.43 (d, 1H), 7.66 (dd, 1H), 7.98 (dd, 1H), 8.04 (d, 1H), 8.12 (d, 1H), 8.87 (d, 1H), 8.89 (d, 1H), 9.23 (s, 1H).

### Example 6A

### Methyl-2-{[1-(2-chlorophenyl)-3-methyl-1H-pyrazol-5-yl]amino}-5-methoxybenzoate

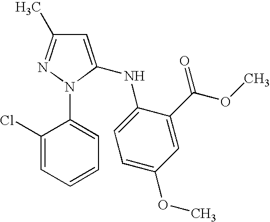

1.00 g (4.185 mmol) of 1-(2-chlorophenyl)-3-methyl-1H-pyrazol-5-amine [Ochiai et al., Chem. Pharm. Bull. 2004, 52, 1098], 1.46 g (5.779 mmol) of methyl-2-bromo-5-methoxybenzoate and 1.43 g (6.741 mmol) potassium phosphate were dissolved in 50 ml absolute toluene. The resultant solution was outgassed with argon. Then 97 mg (0.433 mmol) of palladium(II) acetate and 194 mg (0.650 mmol) of (2-biphenyl)di-tert.-butylphosphine were added. It was stirred for 72 h at the reflux temperature. The mixture was concentrated by evaporation and purified by MPLC (Puriflash Analogix: 40M: isohexane/ethyl acetate=7/1). This gave 211 mg (13% of theor.) of the target compound.

LC-MS (method 3): Rₜ=2.17 min; MS (EIpos): m/z=372 [M+H]⁺.

### Example 7A

### Methyl-2-{[4-bromo-1-(2-chlorophenyl)-3-methyl-1H-pyrazol-5-yl]amino}-5-methoxybenzoate

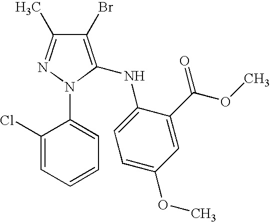

208 mg (0.560 mmol) of example 6A was dissolved in 3 ml acetonitrile and 105 mg (0.588 mmol) of N-bromosuccinimide was added. A temperature of 50° C. was maintained for 10 min and the volatile components were removed in a rotary evaporator. Then it was purified by preparative HPLC (eluent: acetonitrile/water, gradient 10:90→90:10). This gave 95 mg (38% of theor.) of the target compound.

LC-MS (method 3): Rₜ=2.45 min; MS (EIpos): m/z=450 [M]⁺.

¹H-NMR (400 MHz, DMSO-D₆): δ [ppm]=2.25 (s, 3H), 3.68 (s, 3H), 3.81 (s, 3H), 6.56 (d, 1H), 7.06 (dd, 1H), 7.26 (d, 1H), 7.41-7-52 (m, 2H), 7.59-7.65 (m, 2H), 8.75 (s, 1H).

### Example 8A

### Methyl-2-{[4-(quinoxalin-6-yl)-1-(2-chlorophenyl)-3-methyl-1H-pyrazol-5-yl]amino}-5-methoxy-benzoate

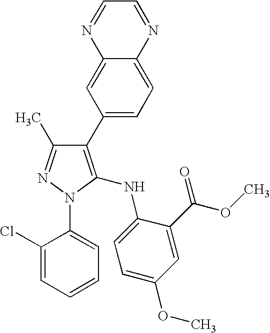

95.0 mg (0.211 mmol) of example 7A and 88.7 mg (0.422 mmol) quinoxalin-6-yl boric acid hydrochloride were dissolved in 15 ml DMF. After adding 200 μl (0.400 mmol) of 2M sodium carbonate solution it was outgassed with argon. 14.6 mg (0.013 mmol) of tetrakis(triphenylphosphine)palladium(0) was added and a temperature of 110° C. was maintained for 1 h. After completion of reaction had been detected by LC-MS, 20 ml ethyl acetate was added and the solid residue was removed by filtration on kieselguhr. The resultant solution was washed with 20 ml water and with 20 ml of saturated aqueous sodium chloride solution. The organic phase was dried over sodium sulfate and the solvent was removed in a rotary evaporator. The raw product was purified by preparative HPLC (eluent: acetonitrile/water, gradient 10:90→90:10). This gave 50 mg (47% of theor.) of the target compound.

LC-MS (method 1): Rₜ=2.47 min; MS (EIpos): m/z=500 [M+H]⁺.

### Example 9A

### 1-(2-Methylphenyl)-3-(trifluoromethyl)-1H-pyrazol-5-amine

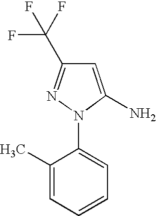

5.00 g (31.5 mmol) of (2-methylphenyl)hydrazine hydrochloride was taken up in 30 ml of 1N hydrochloric acid and 4.55 g (33.4 mmol) of 3-amino-4,4,4-trifluorobut-2-enonitrile [synthesis similar to Krespan, J. Org. Chem. 1969, 34, 42] was added. It was stirred overnight at the reflux temperature, and was then alkalized with 1N sodium hydroxide solution. After extraction with ethyl acetate (2×200 ml), the organic phases were combined and dried over magnesium sulfate. The volatile components were removed in a rotary evaporator and the resultant oil was dried under high vacuum. This gave 6.85 g (90% of theor.) of the target compound.

LC-MS (method 4): Rₜ=1.08 min; MS (EIpos): m/z=242 [M]⁺.

¹H-NMR (400 MHz, DMSO-D₆): δ [ppm]=2.03 (s, 3H), 5.47 (sbr, 2H), 5.72 (s, 1H), 7.30 (d, 1H), 7.35 (dt, 1H), 7.39-7.48 (m, 2H).

### Example 10A

### Methyl-5-chloro-2-{[1-(2-methylphenyl)-3-(trifluoromethyl)-1H-pyrazol-5-yl]amino}benzoate

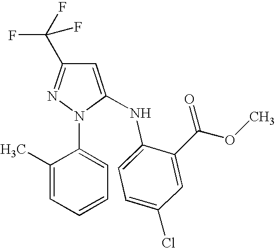

1.50 g (6.22 mmol) of example 9A, 1.86 g (7.46 mmol) of methyl-2-bromo-5-chlorobenzoate and 1.84 g (8.71 mmol) potassium phosphate were dissolved in 75 ml absolute toluene. The resultant solution was outgassed with argon. Then 126 mg (0.560 mmol) of palladium(II) acetate and 251 mg (0.839 mmol) of (2-biphenyl)di-tert.-butylphosphine were added. It was stirred overnight at the reflux temperature. A reaction test showed that reaction was incomplete. Therefore 1.86 g (7.46 mmol) of methyl-2-bromo-5-chlorobenzoate, and 126 mg (0.560 mmol) of palladium(II) acetate and 251 mg (0.839 mmol) of (2-biphenyl)di-tert.-butylphosphine were added again. It was held again overnight at the reflux temperature. The mixture was concentrated by evaporation and purified by MPLC (Puriflash Analogix: 40M: isohexane/ethyl acetate=9/1). This gave 379 mg (12% of theor.) of the target compound at 80% purity (LC-MS fraction).

LC-MS (method 1): Rₜ=3.15 min; MS (EIpos): m/z=410 [M+H]⁺.

### Example 11A

### Methyl-5-chloro-2-{[4-iodine-1-(2-methylphenyl)-3-(trifluoromethyl)-1H-pyrazol-5-yl]amino}benzene carboxylate

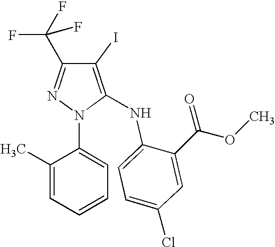

379 mg (0.925 mmol) of example 10A was dissolved in 8 ml acetonitrile and 218 mg (0.971 mmol) of N-iodosuccinimide was added. It was held overnight at the reflux temperature. Checking by LC/MS showed that reaction was incomplete. Therefore 208 mg (0.925 mmol) of N-iodosuccinimide was added and it was stirred overnight at the reflux temperature. Dilute sodium sulfite solution (50 ml) was added and it was extracted with ethyl acetate (2×50 ml). The combined organic phases were dried over magnesium sulfate. The volatile components were removed in a rotary evaporator and it was then purified by preparative HPLC (eluent: acetonitrile/water, gradient 10:90→90:10). This gave 277 mg (45% of theor.) of the target compound.

LC-MS (method 3): Rₜ=2.89 min; MS (EIpos): m/z=536 [M+H]⁺.

### Example 12A

### 1-(2-Ethylphenyl)-3-methyl-1H-pyrazol-5-amine

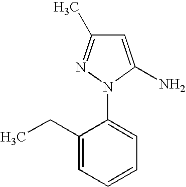

2.000 g (11.584 mmol) of 2-ethylphenylhydrazine hydrochloride was put in 10 ml of 1N hydrochloric acid and 1.008 g (12.279 mmol) of 3-aminocrotonic acid nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. The product was dried under high vacuum. We obtained 2.354 g (94% of theor.) of the target compound.

LC-MS (method 2): Rₜ=1.04 min; MS (EIpos): m/z=202 [M+H]⁺.

### Example 13A

### Methyl-2-{[1-(2-ethylphenyl)-3-methyl-1H-pyrazol-5-yl]amino}-5-methoxybenzoate

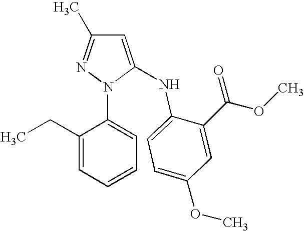

Under an argon atmosphere, 1.260 g (6.260 mmol) of the compound from example 12A was dissolved in 12 ml toluene and 1.279 g (5.217 mmol) of methyl-2-bromo-5-methoxybenzoate, 1.551 g (7.303 mmol) potassium phosphate, 0.210 g (0.704 mmol) of (2-biphenyl)di-tert.-butylphosphine and 0.105 g (0.470 mmol) of palladium(II) acetate were added. The mixture was boiled under reflux overnight. After cooling, it was extracted with ethyl acetate. The combined organic phases were washed with water, dried over sodium sulfate and concentrated in a rotary evaporator at reduced pressure. The residue was purified by preparative HPLC (eluent: acetonitrile/water, gradient 10:90→90:10). We obtained 675 mg (30% of theor.).

LC-MS (method 3): Rₜ=2.30 min; MS (EIpos): m/z=366 [M+H]⁺.

¹H-NMR (400 MHz, DMSO-D₆): δ [ppm]=0.98 (t, 3H), 2.21 (s, 3H), 2.36 (q, 2H), 3.71 (s, 6H), 6.10 (s, 1H), 7.17 (dd, 1H), 7.27-7.36 (m, 4H), 7.42-7-48 (m, 2H), 8.94 (s, 1H).

### Example 14A

### Methyl-2-{[4-bromo-1-(2-ethylphenyl)-3-methyl-1H-pyrazol-5-yl]amino}-5-methoxybenzoate

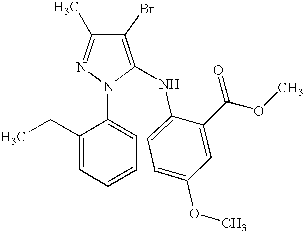

675 mg (1.847 mmol) of the compound from example 13A was dissolved in 10 ml dichloromethane and, at 0-5° C., 264 mg (0.924 mmol) of 1,3-dibromo-5-5-dimethylhydantoin was added. After stirring for 20 min, it was left to warm to room temperature. Dichloromethane was added and the organic phase was washed twice with 10% aqueous sodium thiosulfate solution and saturated aqueous sodium chloride solution and water. The organic phase was dried over sodium sulfate and concentrated in a rotary evaporator at reduced pressure. The resultant residue was purified by preparative HPLC (eluent: acetonitrile/water, gradient 10:90→90:10). We obtained 814 mg (99% of theor.) of the target compound.

LC-MS (method 2): Rₜ=2.70 min; MS (EIpos): m/z=444 [M+H]⁺.

### Example 15A

### Methyl-2-{[4-(quinoxalin-6-yl)-1-(2-ethylphenyl)-3-methyl-1H-pyrazol-5-yl]amino}-5-methoxy-benzoate

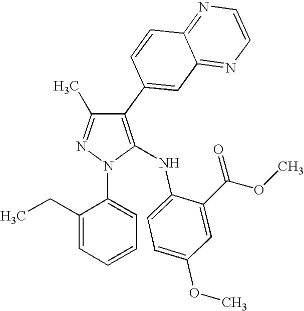

Under an argon atmosphere, 340 mg (0.765 mmol) of the compound from example 14A was dissolved in 1.7 ml dimethylformamide and 484 mg (2.299 mmol) quinoxalin-6-yl boric acid hydrochloride and 1.9 ml of 2N aqueous sodium carbonate solution were added. 53 mg (0.046 mmol) of tetrakis(triphenyl-phosphine)palladium(0) was added and the mixture was heated at 110° C. for 15 min. After cooling, it was extracted with ethyl acetate. The combined organic phases were washed with water, dried over sodium sulfate and concentrated in a rotary evaporator at reduced pressure. The residue was purified by preparative HPLC (eluent: acetonitrile/water, gradient 10:90→90:10). We obtained 494 mg (69% of theor.) of the target compound.

LC-MS (method 3): Rₜ=2.28 min; MS (EIpos): m/z=494 [M+H]⁺.

¹H-NMR (400 MHz, DMSO-D₆): δ [ppm]=1.07 (t, 3H), 2.48 (s, 3H), 2.48 (q, 2H), 3.55 (s, 3H), 3.79 (s, 3H), 6.47 (d, 1H), 6.80 (dd, 1H), 7.13 (d, 1H), 7.24-7.29 (m, 1H), 7.37-7.40 (m, 3H), 7.96 (dd, 1H), 8.02 (d, 1H), 8.10 (d, 1H), 8.88 (d, 1H), 8.90 (d, 1H) 8.91 (s, 1H).

### Example 16A

### Methyl-5-ethyl-2-{[3-methyl-1-(2-methylphenyl)-1H-pyrazol-5-yl]amino}benzoate

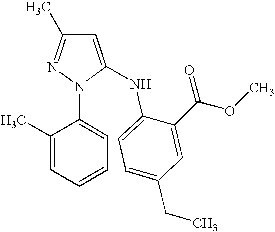

Under an argon atmosphere, 550 mg (2.936 mmol) of 3-methyl-1-(2-methylphenyl)-1H-pyrazol-5-amine (for preparation see WO 2004/050651, p. 30) and 1100 mg (3.523 mmol) of methyl-5-ethyl-2-{[(trifluoromethyl)sulfonyl]oxy}benzoate (for preparation see WO 2004/024081, p. 120) were dissolved in 10 ml anhydrous toluene and 872 mg (4.110 mmol) potassium phosphate and 59 mg (0.264 mmol) of palladium(II) acetate and 118 mg (0.396 mmol) of (2-bisbiphenyl)di-tert.-butylphosphine were added. The reaction mixture was boiled under reflux overnight. After cooling, water was added and it was extracted with ethyl acetate. The combined organic phases were dried over sodium sulfate and filtered on silica gel. The organic phase was concentrated in a rotary evaporator at reduced pressure and the residue was purified on silica gel (eluent: dichloromethane/methanol=100:1). We obtained 636 mg (61% of theor.) of the target compound.

LC-MS (method 4): Rₜ=1.52 min; MS (EIpos): m/z=350 [M+H]⁺.

¹H-NMR (400 MHz, DMSO-D₆): δ [ppm]=1.12 (t, 3H), 2.03 (s, 3H), 2.23 (s, 3H), 2.52 (q, 2H), 3.72 (s, 3H), 6.16 (s, 1H), 7.22 (d, 1H) 7.29-7.40 (m, 5H), 7.66 (d, 1H), 9.14 (s, 1H).

### Example 17A

### Methyl-2-{[4-bromo-3-methyl-1-(2-methylphenyl)-1H-pyrazol-5-yl]amino}-5-ethylbenzoate

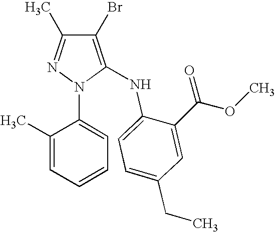

590 mg (1.690 mmol) of the compound from example 16A was dissolved in 9 ml dichloromethane and cooled on an ice bath, so that the temperature was 0-5° C. After adding 242 mg (0.845 mmol) of 1,3-dibromo-5,5-dimethylhydantoin it was stirred for 20 min at 0-5° C. Dichloromethane was added and the organic phase was washed twice with 10% aqueous sodium thiosulfate solution and saturated aqueous sodium chloride solution and water. The organic phase was dried over sodium sulfate and concentrated in a rotary evaporator at reduced pressure. We obtained 708 mg (98% of theor.) of the target compound.

LC-MS (method 3): Rₜ=2.78 min; MS (EIpos): m/z=428 [M+H]⁺.

¹H-NMR (400 MHz, DMSO-D₆): δ [ppm]=1.10 (t, 3H), 2.08 (s, 3H), 2.26 (s, 3H), 2.48 (q, 2H), 3.79 (s, 3H), 6.49 (d, 1H), 7.22-7.34 (m, 5H), 7.61 (d, 1H), 8.85 (s, 1H).

### Example 18A

### Methyl-2-{[4-(quinoxalin-6-yl)-3-methyl-1-(2-methylphenyl)-1H-pyrazol-5-yl]amino}-5-ethylbenzoate

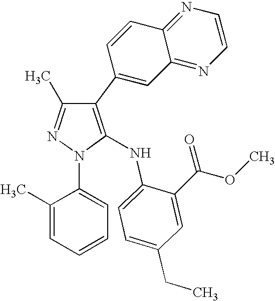

Under an argon atmosphere, 200 mg (0.467 mmol) of the compound from example 17A was dissolved in 5 ml dimethylformamide and 295 mg (1.403 mmol) quinoxalin-6-yl boric acid hydrochloride and 1.2 ml of 2N aqueous sodium carbonate solution were added. 32 mg (0.028 mmol) of tetrakis(triphenylphosphine)palladium(0) was added and the mixture was stirred for 40 min at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 110 mg (49% of theor.) of the target compound.

LC-MS (method 4): Rₜ=1.48 min; MS (EIpos): m/z=478 [M+H]⁺.

¹H-NMR (400 MHz, DMSO-D₆): δ [ppm]=0.97 (t, 3H), 2.18 (s, 3H), 2.34 (q, 2H), 2.48 (s, 3H), 3.79 (s, 3H), 6.41 (d, 1H), 7.01 (dd, 1H), 7.22-7.27 (m, 1H), 7.29-7.34 (m, 3H), 7.42 (d, 1H), 7.48 (d, 1H), 7.97 (dd, 1H), 8.04 (d, 1H), 8.11 (d, 1H), 8.88 (d, 1H), 8.90 (d, 1H) 9.10 (s, 1H).

### Example 19A

### 7-(4,4,5,5-Tetramethyl-1,3,2-dioxaborolan-2-yl)pyrido[2,3-b]pyrazine

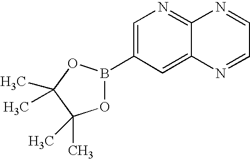

Under an argon atmosphere, 52 mg (0.057 mmol) of tris-(dibenzylidene-acetone)-dipalladium(0) and 38 mg (0.137 mmol) of tricyclohexylphosphine were dissolved in 5.8 ml dioxane. 266 mg (1.047 mmol) of 4,4,4′4′5,5,5′5′-octamethyl-2,2′-bi-1,3,2-dioxaborolan, 200 mg (0.952 mmol) of 7-bromopyrido[2,3-b]pyrazine (described in WO 2006/009734, p. 59) and 140 mg (1.428 mmol) potassium acetate were added and the mixture was stirred overnight at 80° C. After cooling, the reaction mixture was concentrated in a rotary evaporator at reduced pressure, the residue was taken up in cyclohexane/ethyl acetate (v/v=1:1) and filtered on silica gel. The cyclohexane/ethyl acetate phase was discarded and the silica gel was rinsed with dichloromethane/methanol (v/v=10:1). The filtrate was concentrated in a rotary evaporator at reduced pressure. We obtained 178 mg (38% of theor.) of the raw product, at a purity of 52% according to GC-MS. The raw product was reacted further without further purification.

GC-MS (method 8): Rₜ=7.29 min; MS (EIpos): m/z=257 [M]⁺.

### Example 20A

### Methyl-5-methoxy-2-{[3-methyl-1-(2-methylphenyl)-4-(pyrido[2,3-b]pyrazin-7-yl)-1H-pyrazol-5-yl]amino}benzoate

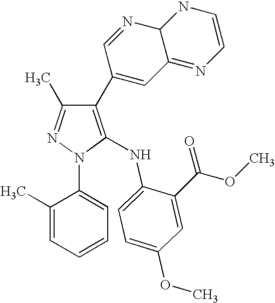

Under an argon atmosphere, 96 mg (0.223 mmol) of methyl-2-{[4-bromo-3-methyl-1-(2-methylphenyl)-1H-pyrazol-5-yl]amino}-5-methoxybenzoate (described in WO 2005/112923, p. 18) and 172 mg (0.348 mmol) of the compound from example 19A were dissolved in 3.8 ml dimethylformamide and 760 μl of 2N aqueous sodium carbonate solution and 15 mg (0.013 mmol) of tetrakis(triphenylphosphine)palladium(0) were added. The mixture was stirred for 20 min at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 61 mg (57% of theor.) of the target compound.

LC-MS (method 1): Rₜ=2.22 min; MS (EIpos): m/z=481 [M+H]⁺.

¹H-NMR (400 MHz, DMSO-D₆): δ [ppm]=2.18 (s, 3H), 3.56 (s, 3H), 3.81 (s, 3H), 6.46 (d, 1H), 6.82 (dd, 1H), 7.14 (d, 1H), 7.24-7.29 (m, 1H), 7.31-7.35 (m, 2H), 7.42 (d, 1H), 8.53 (d, 1H), 8.95 (s, 1H), 9.03 (d, 1H), 9.07 (d, 1H), 9.28 (d, 1H).

### Example 21A

### Methyl-2-{[4-(1H-indazol-5-yl)-3-methyl-1-(2-methylphenyl)-1H-pyrazol-5-yl]amino}-5-methoxybenzoate

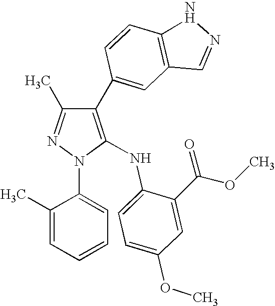

Under an argon atmosphere, 130 mg (0.303 mmol) of methyl-2-{[4-bromo-3-methyl-1-(2-methylphenyl)-1H-pyrazol-5-yl]amino}-5-methoxybenzoate (described in WO2005/112923, p. 18) and 313 mg (0.909 mmol) tert.-butyl-5-(4,4,5,5-tetramethyl-1,3,2-dioxaborolan-2-yl)-1H-indazole-1 carboxylate were dissolved in 3.2 ml dimethylformamide and 760 μl of 2N aqueous sodium carbonate solution and 21 mg (0.018 mmol) of tetrakis(triphenylphosphine)palladium(0) were added. The mixture was stirred at 110° C. overnight. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 20 mg (14% of theor.) of the target compound.

LC-MS (method 4): Rₜ=1.26 min; MS (EIpos): m/z=468 [M+H]⁺.

¹H-NMR (400 MHz, DMSO-D₆): δ [ppm]=2.15 (s, 3H), 2.36 (s, 3H), 3.57 (s, 3H), 3.78 (s, 3H), 6.42 (d, 1H), 6.83 (dd, 1H), 7.11 (d, 1H), 7.20-7.24 (m, 1H), 7.27-7.32 (m, 2H), 7.35 (d, 1H), 7.39 (dd, 1H), 7.47 (d, 1H), 7.77 (s, 1H), 8.02 (s, 1H), 8.78 (s, 1H), 13.01 (s, 1H).

### Example 22A

### Methyl-2-{[4-(quinolin-6-yl)-3-methyl-1-(2-methylphenyl)-1H-pyrazol-5-yl]amino}-5-methoxy-benzoate

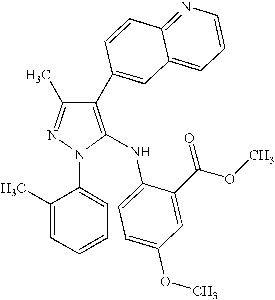

Under an argon atmosphere, 130 mg (0.303 mmol) of methyl-2-{[4-bromo-3-methyl-1-(2-methylphenyl)-1H-pyrazol-5-yl]amino}-5-methoxybenzoate (described in WO 2005/112923, p. 18) and 157 mg (0.909 mmol) of quinolin-6-yl boric acid were dissolved in 3.2 ml dimethylformamide and 760 μl of 2N aqueous sodium carbonate solution and 21 mg (0.018 mmol) of tetrakis(triphenylphosphine)palladium(0) were added. The mixture was stirred for 10 min at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 76 mg (52% of theor.) of the target compound.

LC-MS (method 3): Rₜ=1.96 min; MS (EIpos): m/z=479 [M+H]⁺.

¹H-NMR (400 MHz, DMSO-D₆): δ [ppm]=2.16 (s, 3H), 2.45 (s, 3H), 3.55 (s, 3H), 3.80 (s, 3H), 6.43 (d, 1H), 6.81 (dd, 1H), 7.12 (d, 1H), 7.22-7.27 (m, 1H), 7.30-7.34 (m, 2H), 7.39 (d, 1H), 7.50 (dd, 1H), 7.83 (dd, 1H), 7.93 (d, 1H), 8.01 (d, 1H), 8.27 (d, 1H), 8.84 (dd, 1H), 8.88 (s, 1H).

### Example 23A

### Methyl-2-{[4-(1,3-benzothiazol-5-yl)-3-methyl-1-(2-methylphenyl)-1H-pyrazol-5-yl]amino}-5-methoxybenzoate

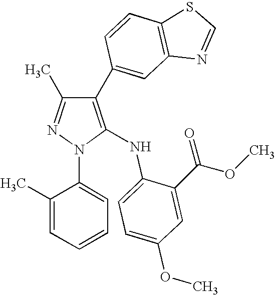

Under an argon atmosphere, 340 mg (0.790 mmol) of methyl-2-{[4-bromo-3-methyl-1-(2-methylphenyl)-1H-pyrazol-5-yl]amino}-5-methoxybenzoate (described in WO 2005/112923, p. 18) and 620 mg (2.374 mmol) of 5-(4,4,5,5-tetramethyl-1,3,2-dioxaborolan-2-yl)-1,3-benzothiazole dissolved in 8.4 ml dimethylformamide and 1.975 ml of 2N aqueous sodium carbonate solution and 55 mg (0.047 mmol) of tetrakis(triphenylphosphine)palladium(0) were added. The mixture was stirred for 15 min at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 60 mg (16% of theor.) of the target compound.

LC-MS (method 4): Rₜ=1.39 min; MS (EIpos): m/z=485 [M+H]⁺.

¹H-NMR (400 MHz, DMSO-D₆): δ [ppm]=2.16 (s, 3H), 2.41 (s, 3H), 3.57 (s, 3H), 3.78 (s, 3H), 6.43 (d, 1H), 6.84 (dd, 1H), 7.13 (d, 1H), 7.20-7.25 (m, 1H), 7.28-7.32 (m, 2H), 7.38 (d, 1H), 7.56 (dd, 1H), 8.10-8.12 (m, 2H), 8.84 (s, 1H), 9.36 (s, 1H).

### Example 24A

### Methyl-5-methoxy-2-{[3-methyl-1-(2-methylphenyl)-4-(naphthalen-2-yl)-1H-pyrazol-5-yl]amino}benzoate

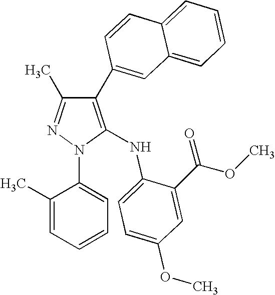

Under an argon atmosphere, 340 mg (0.790 mmol) of methyl-2-{[4-bromo-3-methyl-1-(2-methylphenyl)-1H-pyrazol-5-yl]amino}-5-methoxybenzoate (described in WO 2005/112923, p. 18) and 408 mg (2.374 mmol) naphthalen-2-yl boric acid were dissolved in 8.4 ml dimethylformamide and 1.975 ml of 2N aqueous sodium carbonate solution and 55 mg (0.047 mmol) of tetrakis(triphenylphosphine)palladium(0) were added. The mixture was stirred for 15 min at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 23 mg (6% of theor.) of the target compound.

LC-MS (method 1): Rₜ=3.00 min; MS (EIpos): m/z=478 [M+H]⁺.

### Example 25A

### 4-Bromo-1-(2-methylphenyl)-1H-pyrazol-5-amine

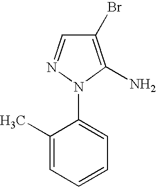

At RT, 6.92 g (43.3 mmol) bromine, dissolved in 5 ml acetic acid, was slowly added dropwise to 7.50 g (43.3 mmol) of 1-(2-methylphenyl)-1H-pyrazol-5-amine (C. Alberti, C. Tironi, Farmaco 1967, 22, 58-75) in 30 ml acetic acid. After stirring for 30 min at RT, 35 ml water was added to the mixture, and it was alkalized with potassium hydroxide powder while cooling on an ice bath. It was extracted twice with 50 ml ethyl acetate each time, the combined organic phases were dried over magnesium sulfate and then concentrated by evaporation. The residue was purified by silica-gel flash chromatography with cyclohexane/ethyl acetate in the ratio 5:1 as eluent. This gave 9.00 g of the target compound (82% of theor.).

LC-MS (method 4): Rₜ=0.94 min; MS (EIpos): m/z=252 [M+H]⁺.

¹H NMR (400 MHz, DMSO-D₆): δ [ppm]=2.04 (s, 3H), 5.19 (s, 2H), 7.24 (d, 1H), 7.29-7.36 (m, 1H), 7.36-7.43 (m, 3H).

### Example 26A

### 4-(Quinoxalin-6-yl)-1-(2-methylphenyl)-1H-pyrazol-5-amine

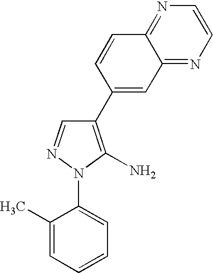

Under an argon atmosphere, 1.50 g (7.14 mmol) quinoxalin-6-yl boric acid hydrochloride, 0.34 g (0.30 mmol) of tetrakis(triphenylphosphine)palladium(0) and 10 ml of saturated aqueous sodium carbonate solution were added to 1.50 g (5.95 mmol) of 4-bromo-1-(2-methylphenyl)-1H-pyrazol-5-amine (example 25A) in 15 ml DMF. The mixture was stirred for 3 h at 110° C. and then added to 150 ml water for processing. It was extracted twice with 50 ml ethyl acetate each time, the combined organic phases were dried over magnesium sulfate and concentrated by evaporation. For purification, it was chromatographed twice on silica gel, first with cyclohexane/ethyl acetate 2:1 followed by pure ethyl acetate as eluents, then with dichloromethane/methanol 50:1, followed by 20:1 as solvent. After concentration of the product fractions by evaporation, we obtained 729 mg of the target compound with an admixture of triphenylphospine oxide (78% according to LC-MS, corresponding to a yield of 32%).

LC-MS (method 2): Rₜ=1.64 min; MS (EIpos): m/z=302 [M+H]⁺.

¹H NMR (400 MHz, DMSO-D₆): δ [ppm]=2.12 (s, 3H), 5.48 (m, 2H), 7.28-7.68 (m, 4H), 7.99 (s, 1H), 8.04 (d, 1H), 8.14 (dd, 1H), 8.18 (d, 1H), 8.82 (d, 1H), 8.89 (d, 1H).

### Example 27A

### Methyl-2-{[4-(quinoxalin-6-yl)-1-(2-methylphenyl)-1H-pyrazol-5-yl]amino}benzoate

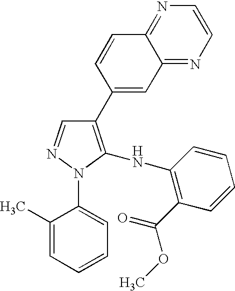

Under an argon atmosphere, 0.09 g (0.22 mmol) of palladium(II) acetate was added to 720 mg (2.39 mmol) of 4-(quinoxalin-6-yl)-1-(2-methylphenyl)-1H-pyrazol-5-amine from example 26A, 617 mg (2.87 mmol) of methyl-2-bromobenzoate, 710 mg (3.35 mmol) potassium phosphate and 96 mg (0.32 mmol) of 2-(di-tert.-butylphosphino)-biphenyl in 7 ml toluene and the reaction mixture was then heated under reflux overnight. After adding first 10 mg (0.04 mmol) of palladium(II) acetate and then a further 10 mg (0.04 mmol) of palladium(II) acetate and 25 mg (0.08 mmol) of 2-(di-tert.-butylphosphino)-biphenyl it was stirred once again in each case overnight under reflux and then processed. For this, the mixture was filtered on Celite and the eluate obtained after washing with ethyl acetate was concentrated by evaporation. After purification by preparative HPLC method 7, 200 mg of the target compound (25% of theor.) was thus obtained.

LC-MS (method 1): Rₜ=2.50 min; MS (EIpos): m/z=436 [M+H]⁺.

¹H NMR (400 MHz, DMSO-D₆): δ [ppm]=2.14 (s, 3H), 3.84 (s, 3H), 6.45 (d, 1H), 6.69 (t, 1H), 7.18-7.29 (m, 2H), 7.33 (d, 2H), 7.44 (d, 1H), 7.77 (dd, 1H), 8.05 (d, 1H), 8.20 (dd, 1H), 8.25 (d, 1H), 8.53 (s, 1H), 8.85 (d, 1H), 8.86 (d, 1H), 9.29 (s, 1H).

### Example 28A

### 4-(Quinoxalin-6-yl)-1-phenyl-1H-pyrazol-5-amine

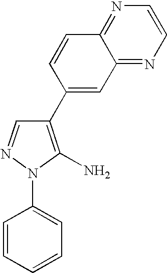

Under an argon atmosphere, 848 mg (4.03 mmol) quinoxalin-6-yl boric acid hydrochloride, 78 mg (0.067 mmol) of tetrakis(triphenylphosphine)palladium(0) and 4 ml of saturated aqueous sodium carbonate solution were added to 800 mg (3.36 mmol) of 4-bromo-1-phenyl-1H-pyrazol-5-amine (U.S. Pat. No. 5,201,938; Arch. Pharm. 1966, 299, 147) in 8 ml DMF. The mixture was stirred for 4 h at 110° C. and was then added to 100 ml water for processing. It was extracted twice with 50 ml ethyl acetate each time, the combined organic phases were filtered on kieselguhr and concentrated by evaporation. For purification, it was chromatographed on silica gel with cyclohexane/ethyl acetate, first in the ratio 5:1, then 2:1, followed by pure ethyl acetate as eluents. After concentration of the product fractions by evaporation, we thus obtained 349 mg of the target compound with an admixture of triphenylphosphine oxide (81% according to LC-MS, corresponding to a yield of 29%).

LC-MS (method 4): Rₜ=0.89 min; MS (EIpos): m/z=288 [M+H]⁺.

¹H NMR (400 MHz, DMSO-D₆): δ [ppm]=5.71 (s, 2H), 7.43 (t, 1H), 7.52-7.66 (m, 4H), 7.99 (s, 1H), 8.06 (d, 1H), 8.12 (dd, 1H), 8.19 (d, 1H), 8.84 (d, 1H), 8.90 (d, 1H).

### Example 29A

### Methyl-2-{[4-(quinoxalin-6-yl)-1-phenyl-1H-pyrazol-5-yl]amino}benzoate

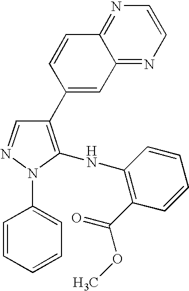

Under an argon atmosphere, 24 mg (0.11 mmol) of palladium(II) acetate was added to 340 mg (1.18 mmol) of 4-(quinoxalin-6-yl)-1-phenyl-1H-pyrazol-5-amine from example 28A, 254 mg (1.18 mmol) of methyl-2-bromobenzoate, 352 mg (1.66 mmol) potassium phosphate and 48 mg (0.16 mmol) of 2-(di-tert.-butylphosphino)-biphenyl in 4 ml toluene and the reaction mixture was then heated overnight under reflux. After adding a further 10 mg (0.04 mmol) of palladium(II) acetate it was stirred twice more overnight under reflux and then processed. For this, the mixture was added to water and then extracted twice with ethyl acetate. After drying the organic phases over magnesium sulfate and concentrating the solvent by evaporation, the residue was purified by silica-gel flash chromatography first with in the ratio 5:1, then 2:1, followed by pure ethyl acetate as eluents. This gave 100 mg of the target compound (25% of theor.).

LC-MS (method 3): Rₜ=2.11 min; MS (EIpos): m/z=422 [M+H]⁺.

¹H NMR (400 MHz, DMSO-D₆): δ [ppm]=3.91 (s, 3H), 6.28 (d, 1H), 6.68 (t, 1H), 7.18 (td, 1H), 7.34 (t, 1H), 7.44 (t, 2H), 7.66 (d, 2H), 7.82 (dd, 1H), 8.05 (d, 1H), 8.19 (dd, 1H), 8.28 (d, 1H), 8.53 (s, 1H), 8.85 (d, 1H), 8.87 (d, 1H), 9.47 (s, 1H).

### Example 30A

### 6-Bromo-7-fluoroquinoxaline

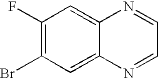

3.300 g (16.095 mmol) of 4-bromo-5-fluorobenzene-1,2-diamine (described in WO 2008/021851, p. 75-76) was dissolved in 160 ml ethanol and 1.933 g (16.095 mmol) trans-2,3-dihydroxy-1,4-dioxane was added. It was stirred for 2 h at room temperature and the reaction mixture was concentrated to 3/4 by evaporation in a rotary evaporator. The resultant precipitate was filtered off and dried under high vacuum. We obtained 2.950 g (81% of theor.) of the target compound.

LC-MS (method 4): Rₜ=1.00 min; MS (EIpos): m/z=227 [M+H]⁺.

¹H-NMR (400 MHz, DMSO-D₆): δ [ppm]=8.11 (d, 1H), 8.58 (d, 1H), 8.98-9.03 (m, 2H).

### Example 31A

### 6-Fluoro-7-(4,4,5,5-tetramethyl-1,3,2-dioxaborolan-2-yl)quinoxaline

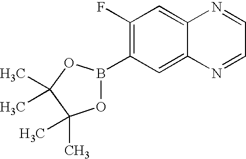

Under an argon atmosphere, 714 mg (0.780 mmol) of tris-(dibenzylidene-acetone)-dipalladium(0) and 525 mg (1.871 mmol) of tricyclohexylphosphine were dissolved in 80 ml dioxane. 3630 mg (14.293 mmol) of 4,4,4′4′5,5,5′5′-octamethyl-2,2′-bi-1,3,2-dioxaborolan, 2950 mg (12.993 mmol) of the compound from example 30A and 1913 mg (19.490 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 6530 mg of the raw product (purity around 30% according to GC-MS), which was reacted further without further purification.

GC-MS (method 6): Rₜ=6.57 min; MS (EIpos): m/z=274 [M]⁺.

### Example 32A

### Methyl-2-{[4-(7-fluoroquinoxalin-6-yl)-3-methyl-1-(2-methylphenyl)-1H-pyrazol-5-yl]amino}-5-methoxybenzoate

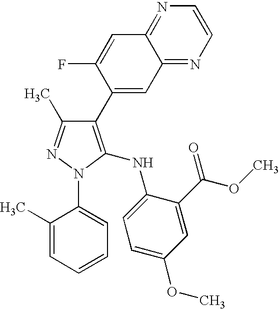

Under an argon atmosphere, 350 mg (0.813 mmol) of methyl-2-{[4-bromo-3-methyl-1-(2-methylphenyl)-1H-pyrazol-5-yl]amino}-5-methoxybenzoate (described in WO 2005/112923, p. 18) and 3000 mg (approx. 3.283 mmol) of the compound from example 31A were dissolved in 9.000 ml dimethylformamide and 2.033 ml of 2N aqueous sodium carbonate solution and 56 mg (0.049 mmol) of tetrakis(triphenylphosphine)palladium(0) were added. The mixture was stirred for 3 h at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 164 mg (purity 40%, 16% of theor.) of the target compound.

LC-MS (method 4): Rₜ=1.35 min; MS (EIpos): m/z=498 [M+H]⁺.

### Example 33A

### 4-Bromo-5-chloro-2-nitroaniline

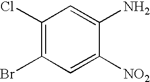

6.00 g (34.768 mmol) of 5-chloro-2-nitroaniline and 6.06 g (34.073 mmol) of N-bromosuccinimide were dissolved in 240 ml acetic acid. The mixture was boiled under reflux for 45 min. After cooling, the reaction mixture was added to 1.5 l water. The resultant precipitate was filtered off and dried under high vacuum. The residue was purified by preparative HPLC (eluent: acetonitrile/water, gradient 10:90→90:10). We obtained 6.75 g (75% of theor.) of the target compound.

LC-MS (method 4): Rₜ=1.19 min; MS (EImin): m/z=249 [M−H]⁺.

¹H-NMR (400 MHz, DMSO-D₆): δ [ppm]=7.29 (s, 1H), 7.62 (sbr, 2H), 8.24 (s, 1H).

### Example 34A

### 4-Bromo-5-chlorobenzene-1,2-diamine

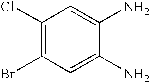

5.00 g (19.883 mmol) of the compound from example 33A was dissolved in 120 ml ethanol and 17.95 g (79.531 mmol) of tin(II) chloride dihydrate was added. The mixture was stirred overnight at 70° C. After cooling, water was added, it was made weakly alkaline with saturated aqueous sodium hydrogen carbonate solution and extracted three times with ethyl acetate. The combined organic phases were dried over sodium sulfate and concentrated in a rotary evaporator at reduced pressure. The residue was dried under high vacuum. We obtained 4.25 g (purity 77%, 74% of theor.) of the target compound.

LC-MS (method 3): Rₜ=1.36 min; MS (EIpos): m/z=221 [M+H]⁺.

¹H-NMR (400 MHz, DMSO-D₆): δ [ppm]=4.85 (s, 2H), 4.89 (s, 2H), 6.64 (s, 1H), 6.75 (s, 1H).

### Example 35A

### 6-Bromo-7-chloroquinoxaline

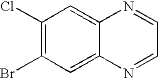

4.25 g (approx. 14.775 mmol) of the compound from example 34A was dissolved in 200 ml ethanol and 2.30 g (19.188 mmol) of trans-2,3-dihydroxy-1,4-dioxane was added. It was stirred overnight at room temperature and the mixture was left to stand over the weekend. The product that crystallized out was filtered off and dried under high vacuum. We obtained 3.33 g (94% of theor.) of the target compound.

LC-MS (method 3): Rₜ=1.77 min; MS (EIpos): m/z=243 [M+H]⁺.

¹H-NMR (400 MHz, DMSO-D₆): δ [ppm]=8.43 (s, 1H), 8.59 (s, 1H), 9.01 (d, 1H), 9.03 (d, 1H).

### Example 36A

### 6-Chloro-7-(4,4,5,5-tetramethyl-1,3,2-dioxaborolan-2-yl)quinoxaline

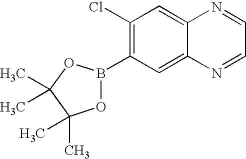

Under an argon atmosphere, 741 mg (0.809 mmol) of tris-(dibenzylidene-acetone)-dipalladium(0) and 545 mg (1.943 mmol) of tricyclohexylphosphine were dissolved in 80 ml dioxane. 3769 mg (14.840 mmol) of 4,4,4′4′5,5,5′5′-octamethyl-2,2′-bi-1,3,2-dioxaborolan, 3285 mg (13.491 mmol) of the compound from example 35A and 1986 mg (20.236 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 7300 mg of the raw product (purity around 18% according to GC-MS), which was reacted further without further purification.

GC-MS (method 6): Rₜ=7.22 min; MS (EIpos): m/z=290 [M]⁺.

### Example 37A

### Methyl-2-{[4-(7-chloroquinoxalin-6-yl)-3-methyl-1-(2-methylphenyl)-1H-pyrazol-5-yl]amino}-5-methoxybenzoate

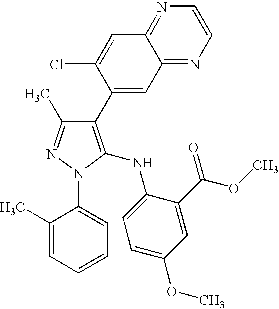

Under an argon atmosphere, 350 mg (0.813 mmol) of methyl-2-{[4-bromo-3-methyl-1-(2-methylphenyl)-1H-pyrazol-5-yl]amino}-5-methoxybenzoate (described in WO 2005/112923, p. 18) and 4000 mg (approx. 2.478 mmol) of the compound from example 36A were dissolved in 11.000 ml dimethylformamide and 2.033 ml of 2N aqueous sodium carbonate solution and 56 mg (0.049 mmol) of tetrakis(triphenylphosphine)palladium(0) were added. The mixture was stirred for 3 h at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 36 mg (purity 81%, 7% of theor.) of the target compound.

LC-MS (method 1): Rₜ=2.66 min; MS (EIpos): m/z=514 [M+H]⁺.

### Example 38A

### 4-Bromo-5-methyl-2-nitroaniline

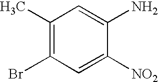

5.00 g (32.861 mmol) of 5-methyl-2-nitroaniline and 5.73 g (32.204 mmol) of N-bromosuccinimide were dissolved in 225 ml acetic acid. The mixture was boiled under reflux for 90 min. After cooling, the reaction mixture was added to 1.5 l water. The resultant precipitate was filtered off and dried under high vacuum. We obtained 6.65 g (84% of theor.) of the target compound.

LC-MS (method 1): Rₜ=2.23 min; MS (EIpos): m/z=231 [M+H]⁺.

¹H-NMR (400 MHz, DMSO-D₆): δ [ppm]=2.27 (s, 3H), 6.98 (s, 1H), 7.48 (sbr, 2H), 8.09 (s, 1H).

### Example 39A

### 4-Bromo-5-methylbenzene-1,2-diamine

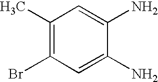

4.00 g (17.312 mmol) of the compound from example 38A was dissolved in 100 ml ethanol and 15.63 g (69.248 mmol) of tin(II) chloride dihydrate was added. The mixture was stirred overnight at 70° C. After cooling, water was added, it was made weakly alkaline with saturated aqueous sodium hydrogen carbonate solution and extracted three times with ethyl acetate. The combined organic phases were dried over sodium sulfate and concentrated in a rotary evaporator at reduced pressure. The residue was dried under high vacuum. We obtained 3.23 g (89% of theor.) of the target compound.

LC-MS (method 1): Rₜ=1.07 min; MS (EIpos): m/z=201 [M+H]⁺.

¹H-NMR (400 MHz, DMSO-D₆): δ [ppm]=2.08 (s, 3H), 4.51 (s, 2H), 4.52 (s, 2H), 6.43 (s, 1H), 6.66 (s, 1H).

### Example 40A

### 6-Bromo-7-methylquinoxaline

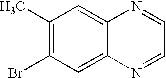

1.00 g (4.973 mmol) of the compound from example 39A was dissolved in 50 ml ethanol and 0.59 g (4.973 mmol) trans-2,3-dihydroxy-1,4-dioxane was added. It was stirred overnight at room temperature. The reaction mixture was concentrated in a rotary evaporator at reduced pressure, taken up in ethyl acetate and purified on silica gel. We obtained 994 mg (84% of theor.) of the target compound.

LC-MS (method 3): Rₜ=1.65 min; MS (EIpos): m/z=224 [M+H]⁺.

¹H-NMR (400 MHz, DMSO-D₆): δ [ppm]=2.60 (s, 3H), 8.11 (s, 1H), 8.39 (s, 1H), 8.92 (d, 1H), 8.96 (d, 1H).

### Example 41A

### 6-Methyl-7-(4,4,5,5-tetramethyl-1,3,2-dioxaborolan-2-yl)quinoxaline

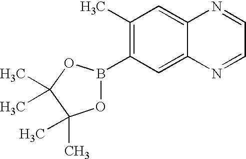

Under an argon atmosphere, 243 mg (0.265 mmol) of tris-(dibenzylidene-acetone)-dipalladium(0) and 179 mg (0.637 mmol) of tricyclohexylphosphine were dissolved in 30 ml dioxane. 1236 mg (4.865 mmol) of 4,4,4′4′5,5,5′5′-octamethyl-2,2′-bi-1,3,2-dioxaborolan, 987 mg (4.423 mmol) of the compound from example 40A and 651 mg (6.635 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 2321 mg of the raw product (purity 42% according to

LC-MS, 82% of theor.), which was reacted further without further purification.

GC-MS (method 6): Rₜ=7.01 min; MS (EIpos): m/z=270 [M]⁺.

### Example 42A

### Methyl-5-methoxy-2-{[3-methyl-4-(7-methylquinoxalin-6-yl)-1-(2-methylphenyl)-1H-pyrazol-5-yl]amino}benzoate

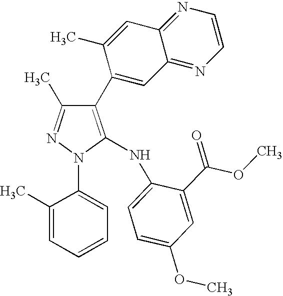

Under an argon atmosphere, 350 mg (0.813 mmol) of methyl-2-{[4-bromo-3-methyl-1-(2-methylphenyl)-1H-pyrazol-5-yl]amino}-5-methoxybenzoate (described in WO 2005/112923, p. 18) and 2321 mg (approx. 3.608 mmol) of the compound from example 41A were dissolved in 20.000 ml dimethylformamide and 2.033 ml of 2N aqueous sodium carbonate solution and 56 mg (0.049 mmol) of tetrakis(triphenylphosphine)palladium(0) were added. The mixture was stirred for 3 h at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 36 mg (purity 88%, 31% of theor.) of the target compound.

LC-MS (method 3): Rₜ=2.18 min; MS (EIpos): m/z=494 [M+H]⁺.

### Example 43A

### 4-Bromo-3-chloro-2-nitroaniline

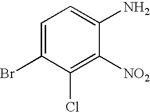

5.00 g (28.973 mmol) of 3-chloro-2-nitroaniline and 5.05 g (28.394 mmol) of N-bromosuccinimide were dissolved in 250 ml acetic acid. The mixture was boiled under reflux for 45 min. After cooling, the reaction mixture was added to 1.5 l water. The resultant precipitate was filtered off and dried under high vacuum. We obtained 5.25 g (72% of theor.) of the target compound.

LC-MS (method 9): Rₜ=2.16 min; MS (EImin): m/z=251 [M−H]⁺.

¹H-NMR (400 MHz, DMSO-D₆): δ [ppm]=6.40 (sbr, 2H), 6.83 (d, 1H), 7.56 (d, 1H).

### Example 44A

### 4-Bromo-3-chlorobenzene-1,2-diamine

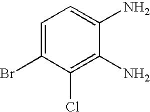

5.25 g (20.877 mmol) of the compound from example 43A was dissolved in 120 ml ethanol and 18.84 g (83.508 mmol) of tin(II) chloride dihydrate was added. The mixture was stirred overnight at 70° C. After cooling, water was added, it was made weakly alkaline with saturated aqueous sodium hydrogen carbonate solution and extracted three times with ethyl acetate. The combined organic phases were dried over sodium sulfate and concentrated in a rotary evaporator at reduced pressure. The residue was dried under high vacuum. We obtained 4.40 g (purity 74%, 70% of theor.) of the target compound.

LC-MS (method 3): Rₜ=1.42 min; MS (EIpos): m/z=221 [M+H]⁺.

### Example 45A

### 6-Bromo-5-choroquinoxaline

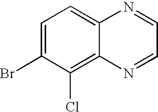

4.40 g (approx. 14.700 mmol) of the compound from example 44A was dissolved in 200 ml ethanol and 2.39 g (19.865 mmol) trans-2,3-dihydroxy-1,4-dioxane was added. It was stirred overnight at room temperature. The reaction mixture was concentrated by evaporation to 2/3, the product that crystallized out was filtered off, washed with a little ethanol and dried under high vacuum. We obtained 3.33 g (89% of theor.) of the target compound.

LC-MS (method 4): Rₜ=1.04 min; MS (EIpos): m/z=243 [M+H]⁺.

¹H-NMR (400 MHz, DMSO-D₆): δ [ppm]=8.04 (d, 1H), 8.19 (d, 1H), 9.09 (d, 1H), 9.10 (d, 1H).

### Example 46A

### 5-Chloro-6-(4,4,5,5-tetramethyl-1,3,2-dioxaborolan-2-yl)quinoxaline

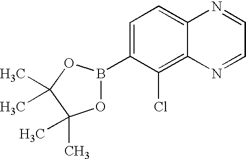

Under an argon atmosphere, 722 mg (0.789 mmol) of tris-(dibenzylidene-acetone)-dipalladium(0) and 531 mg (1.892 mmol) of tricyclohexylphosphine were dissolved in 80 ml dioxane. 3671 mg (14.456 mmol) of 4,4,4′4′5,5,5′5′-octamethyl-2,2′-bi-1,3,2-dioxaborolan, 3200 mg (13.142 mmol) of the compound from example 45A and 1935 mg (19.713 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 7520 mg of the raw product (purity 57% according to GC-MS), which was reacted further without further purification.

GC-MS (method 6): Rₜ=7.54 min; MS (EIpos): m/z=290 [M]⁺.

### Example 47A

### Methyl-2-{[4-(5-chloroquinoxalin-6-yl)-3-methyl-1-(2-methylphenyl)-1H-pyrazol-5-yl]amino}-5-methoxybenzoate

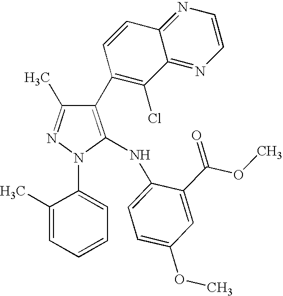

Under an argon atmosphere, 350 mg (0.813 mmol) of methyl-2-{[4-bromo-3-methyl-1-(2-methylphenyl)-1H-pyrazol-5-yl]amino}-5-methoxybenzoate (described in WO 2005/112923, p. 18) and 3000 mg (approx. 5.885 mmol) of the compound from example 46A were dissolved in 10.000 ml dimethylformamide and 2.033 ml of 2N aqueous sodium carbonate solution and 56 mg (0.049 mmol) of tetrakis(triphenylphosphine)palladium(0) were added. The mixture was stirred for 3 h at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water, gradient 10:90→90:10). We obtained 172 mg (purity 50%, 21% of theor.) of the target compound.

LC-MS (method 3): Rₜ=2.19 min; MS (EIpos): m/z=514 [M+H]⁺.

### Example 48A

N-(5-Bromopyridin-2-yl)-N′-hydroxyimidoformamide

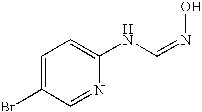

10.00 g (57.797 mmol) of 2-amino-5-bromopyridine was suspended in 20 ml isopropanol and 10.05 ml (75.137 mmol) of dimethylformamide-dimethylacetal was added dropwise at room temperature. The mixture was boiled under reflux for 3 h. It was cooled to 50° C., 5.221 g (75.137 mmol) of hydroxylamine hydrochloride was added and it was stirred at 50° C. overnight. After cooling, the reaction mixture was concentrated in a rotary evaporator. The raw product was purified by preparative HPLC (eluent: acetonitrile/water, gradient 10:90→90:10). We obtained 10.20 g (82% of theor.) of the target compound.

LC-MS (method 4): Rₜ=0.78 min; MS (EIpos): m/z=216 [M+H]⁺.

### Example 49A

### 6-Bromo[1,2,4]triazolo[1,5-a]pyridine

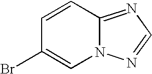

6.00 g (27.772 mmol) of the compound from example 48A was suspended in 60 ml THF and cooled on an ice bath. 6.42 g (30.549 mmol) of trifluoroacetic anhydride was added dropwise within 5-10 min, so that the internal temperature did not exceed 20° C. The ice bath was removed and it was stirred for 3.5 h at room temperature. After adding 150 ml of 5% aqueous sodium hydrogen carbonate solution, it was extracted three times with tert.-butylmethyl ether. The combined organic phases were washed with a little 5% aqueous sodium hydrogen carbonate solution, dried over sodium sulfate and concentrated in a rotary evaporator. We obtained 4.60 g (84% of theor.) of the target compound.

LC-MS (method 1): Rₜ=1.15 min; MS (EIpos): m/z=198 [M+H]⁺.

¹H-NMR (400 MHz, DMSO-D₆): δ [ppm]=7.82 (dd, 1H), 7.86 (d, 1H), 8.54 (s, 1H), 9.41 (m, 1H).

### Example 50A

### Methyl-5-methoxy-2-{[3-methyl-1-(2-methylphenyl)-4-([1,2,4]triazolo[1,5-a]pyridin-6-yl)-1H-pyrazol-5-yl]amino}benzoate

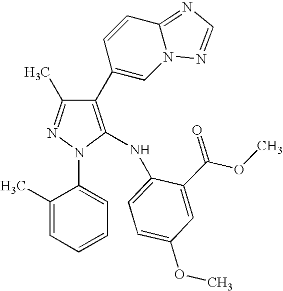

Under an argon atmosphere, 277 mg (0.303 mmol) of tris-(dibenzylidene-acetone)-dipalladium(0) and 224 mg (0.727 mmol) of tricyclohexylphosphine were dissolved in 30 ml dioxane. 1410 mg (5.555 mmol) of 4,4,4′4′5,5,5′5′-octamethyl-2,2′-bi-1,3,2-dioxaborolan, 1000 mg (5.050 mmol) of the compound from example 49A and 743 mg (7.575 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 2520 mg of 6-(4,4,5,5-tetramethyl-1,3,2-dioxaborolan-2-yl)[1,2,4]triazolo[1,5-a]pyridine as raw product, which was reacted further without further purification.

Under an argon atmosphere, 350 mg (0.813 mmol) of methyl-2-{[4-bromo-3-methyl-1-(2-methylphenyl)-1H-pyrazol-5-yl]amino}-5-methoxybenzoate (described in WO 2005/112923, p. 18) and 2279 mg of 6-(4,4,5,5-tetramethyl-1,3,2-dioxaborolan-2-yl)[1,2,4]triazolo[1,5-a]pyridine (raw product) were dissolved in 13.000 ml dimethylformamide and 2.033 ml of aqueous 2N sodium carbonate solution and 56 mg (0.049 mmol) of tetrakis(triphenylphosphine)palladium(0) were added. The mixture was stirred for 2 h at 110° C. After cooling, water was added and it was extracted with ethyl acetate. The combined organic phases were dried over sodium sulfate and concentrated in a rotary evaporator at reduced pressure. The residue was purified by preparative HPLC (eluent: acetonitrile/water+0.5% TFA, gradient 10:90→90:10). We obtained 198 mg (52% of theor.) of the target compound.

LC-MS (method 4): Rₜ=1.20 min; MS (EIpos): m/z=469 [M+H]⁺.

¹H-NMR (400 MHz, DMSO-D₆): δ [ppm]=2.16 (s, 3H), 2.40 (s, 3H), 3.57 (s, 3H), 3.79 (s, 3H), 6.43 (d, 1H), 6.85 (dd, 1H), 7.13 (d, 1H), 7.21-7.26 (m, 1H), 7.29-7.35 (m, 3H), 7.75 (dd, 1H), 7.81 (d, 1H), 8.47 (s, 1H), 8.83 (s, 1H), 9.03 (sbr, 1H).

### Example 51A

### 1-(2-Methoxyphenyl)-3-methyl-1H-pyrazol-5-amine

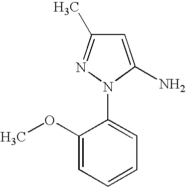

2.000 g (11.453 mmol) of 2-methoxyphenylhydrazine hydrochloride was put in 10 ml of 1N hydrochloric acid and 0.997 g (12.140 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. The product was dried under high vacuum. We obtained 2.430 g (purity according to LC-MS 91%, 95% of theor.) of the target compound.

LC-MS (method 3): Rₜ=0.31 min; MS (EIpos): m/z=204 [M+H]⁺.

### Example 52A

### Methyl-5-methoxy-2-{[1-(2-methoxyphenyl)-3-methyl-1H-pyrazol-5-yl]amino}benzoate

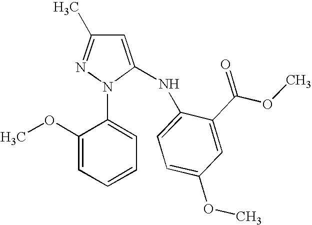

Under an argon atmosphere, 2.300 g (approx. 10.298 mmol) of the compound from example 51A was dissolved in 20 ml toluene and 2.103 g (8.585 mmol) of methyl-2-bromo-5-methoxybenzoate, 2.551 g (12.014 mmol) potassium phosphate, 0.346 g (1.159 mmol) of (2-biphenyl)di-tert.-butylphosphine and 0.173 g (0.772 mmol) of palladium(II) acetate were added. The mixture was boiled under reflux overnight. After cooling, it was extracted with ethyl acetate. The combined organic phases were washed with water, dried over sodium sulfate and concentrated in a rotary evaporator at reduced pressure. The residue was purified by preparative HPLC (eluent: acetonitrile/water, gradient 10:90→90:10). We obtained 135 mg (4% of theor.).

LC-MS (method 4): Rₜ=1.28 min; MS (EIpos): m/z=368 [M+H]⁺.

¹H-NMR (400 MHz, DMSO-D₆): δ [ppm]=2.20 (s, 3H), 3.70 (s, 3H), 3.80 (s, 3H), 3.84 (s, 3H), 6.07 (s, 1H), 7.04 (dt, 1H), 7.11 (dd, 1H), 7.16-7.24 (m, 2H), 7.30 (d, 1H), 7.34 (dd, 1H), 7.41-7.46 (m, 1H), 9.14 (sbr, 1H).

### Example 53A

### Methyl-2-{[4-bromo-1-(2-methoxyphenyl)-3-methyl-1H-pyrazol-5-yl]amino}-5-methoxybenzoate

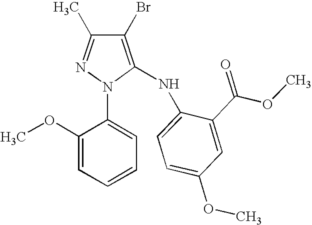

132 mg (0.359 mmol) of the compound from example 52A was dissolved in 2 ml dichloromethane and, at 0-5° C., 51 mg (0.180 mmol) of 1,3-dibromo-5,5-dimethylhydantoin was added. After stirring for 20 min, it was left to warm to room temperature. Dichloromethane was added and the organic phase was washed twice with 10% aqueous sodium thiosulfate solution and saturated aqueous sodium chloride solution and water. The organic phase was dried over sodium sulfate and concentrated in a rotary evaporator at reduced pressure. We obtained 148 mg (92% of theor.) of the target compound.

LC-MS (method 4): Rₜ=1.41 min; MS (EIpos): m/z=446 [M+H]⁺.

¹H-NMR (400 MHz, DMSO-D₆): δ [ppm]=2.23 (s, 3H), 3.67 (s, 3H), 3.82 (s, 3H), 3.83 (s, 3H), 7.02 (dt, 1H), 7.06 (dd, 1H), 7.18 (d, 1H), 7.27 (d, 1H), 7.36 (dd, 1H), 7.39-7.44 (m, 1H), 8.88 (sbr, 1H).

### Example 54A

### Methyl-2-{[4-(quinoxalin-6-yl)-1-(2-methoxyphenyl)-3-methyl-1H-pyrazol-5-yl]amino}-5-methoxybenzoate

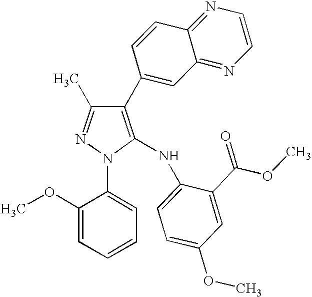

Under an argon atmosphere, 145 mg (0.325 mmol) of the compound from example 53A was dissolved in 3.5 ml dimethylformamide and 205 mg (0.976 mmol) quinoxalin-6-yl boric acid hydrochloride and 0.8 ml of 2N aqueous sodium carbonate solution were added. 23 mg (0.019 mmol) of tetrakis(triphenylphosphine)palladium(0) was added and the mixture was heated at 110° C. for 15 min. After cooling, it was extracted with ethyl acetate. The combined organic phases were washed with water, dried over sodium sulfate and concentrated in a rotary evaporator at reduced pressure. The residue was purified by preparative HPLC (eluent: acetonitrile/water, gradient 10:90→90:10). We obtained 94 mg (58% of theor.) of the target compound.

LC-MS (method 4): Rₜ=1.26 min; MS (EIpos): m/z=496 [M+H]⁺.

¹H-NMR (400 MHz, DMSO-D₆): δ [ppm]=2.46 (s, 3H), 3.53 (s, 3H), 3.84 (s, 3H), 3.86 (s, 3H), 6.43 (d, 1H), 6.73 (dd, 1H), 7.03 (dt, 1H), 7.14 (d, 1H), 7.20 (d, 1H), 7.39-7.44 (m, 2H), 7.91 (dd, 1H), 8.00 (d, 1H), 8.05 (d, 1H), 8.87 (d, 1H), 8.89 (d, 1H) 9.10 (sbr, 1H).

### Example 55A

### 1-(2-Ethoxyphenyl)-3-methyl-1H-pyrazol-5-amine

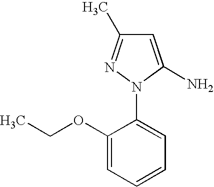

2.000 g (10.601 mmol) of 2-ethoxyphenylhydrazine hydrochloride was put in 10 ml of 1N hydrochloric acid and 0.923 g (11.237 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. The product was dried under high vacuum. We obtained 2.100 g (purity according to LC-MS 91%, 83% of theor.) of the target compound.

LC-MS (method 3): Rₜ=0.57 min; MS (EIpos): m/z=218 [M+H]⁺.

### Example 56A

### Methyl-5-methoxy-2-{[1-(2-ethoxyphenyl)-3-methyl-1H-pyrazol-5-yl]amino}benzoate

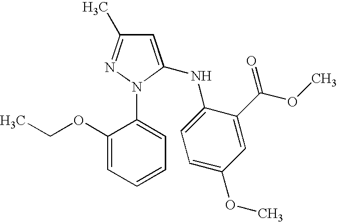

Under an argon atmosphere, 2.000 g (approx. 8.377 mmol) of the compound from example 55A was dissolved in 16 ml toluene and 1.711 g (6.980 mmol) of methyl-2-bromo-5-methoxybenzoate, 2.075 g (9.773 mmol) potassium phosphate, 0.281 g (0.942 mmol) of (2-biphenyl)di-tert.-butylphosphine and 0.141 g (0.628 mmol) of palladium(II) acetate were added. The mixture was boiled under reflux overnight. After cooling, it was extracted with ethyl acetate. The combined organic phases were washed with water, dried over sodium sulfate and concentrated in a rotary evaporator at reduced pressure. The residue was purified by preparative HPLC (eluent: acetonitrile/water, gradient 10:90→90:10). We obtained 1.323 mg (41% of theor.).

LC-MS (method 4): Rₜ=1.34 min; MS (EIpos): m/z=382 [M+H]⁺.

¹H-NMR (400 MHz, DMSO-D₆): δ [ppm]=1.20 (t, 3H), 2.21 (s, 3H), 3.70 (s, 3H), 3.79 (s, 3H), 4.10 (q, 2H), 6.09 (s, 1H), 7.03 (dt, 1H), 7.11 (dd, 1H), 7.17-7.21 (m, 2H), 7.30 (d, 1H), 7.33 (dd, 1H), 7.38-7.42 (m, 1H), 9.13 (sbr, 1H).

### Example 57A

### Methyl-2-{[4-bromo-1-(2-ethoxyphenyl)-3-methyl-1H-pyrazol-5-yl]amino}-5-methoxybenzene carboxylate

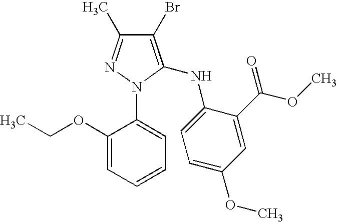

1280 mg (3.356 mmol) of the compound from example 56A was dissolved in 18 ml dichloromethane and, at 0-5° C., 478 mg (1.678 mmol) of 1,3-dibromo-5,5-dimethylhydantoin was added. After stirring for 20 min, it was left to warm to room temperature. Dichloromethane was added and the organic phase was washed twice with 10% aqueous sodium thiosulfate solution and saturated aqueous sodium chloride solution and water. The organic phase was dried over sodium sulfate and concentrated in a rotary evaporator at reduced pressure. We obtained 1423 mg (92% of theor.) of the target compound.

LC-MS (method 4): Rₜ=1.48 min; MS (EIpos): m/z=460 [M+H]⁺.

¹H-NMR (400 MHz, DMSO-D₆): δ [ppm]=1.30 (t, 3H), 2.24 (s, 3H), 3.67 (s, 3H), 3.83 (s, 3H), 4.10 (q, 2H), 6.50 (d, 1H), 7.00 (dt, 1H), 7.05 (dd, 1H), 7.17 (d, 1H), 7.26 (d, 1H), 7.35 (dd, 1H), 7.37-7.41 (m, 1H), 8.87 (sbr, 1H).

### Example 58A

### Methyl-2-{[4-(quinoxalin-6-yl)-1-(2-ethoxyphenyl)-3-methyl-1H-pyrazol-5-yl]amino}-5-methoxybenzoate

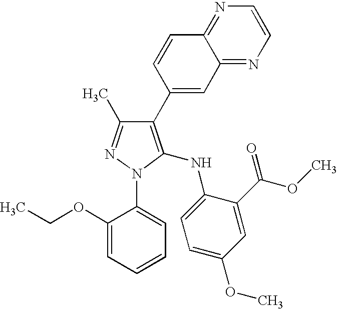

Under an argon atmosphere, 350 mg (0.760 mmol) of the compound from example 57A was dissolved in 8 ml dimethylformamide and 481 mg (2.285 mmol) quinoxalin-6-yl boric acid hydrochloride and 1.9 ml of 2N aqueous sodium carbonate solution were added. 53 mg (0.046 mmol) of tetrakis(triphenylphosphine)palladium(0) was added and the mixture was heated at 110° C. for 45 min. After cooling, it was extracted with ethyl acetate. The combined organic phases were washed with water, dried over sodium sulfate and concentrated in a rotary evaporator at reduced pressure. The residue was purified by preparative HPLC (eluent: acetonitrile/water, gradient 10:90→90:10). We obtained 215 mg (55% of theor.) of the target compound.

LC-MS (method 4): Rₜ=1.32 min; MS (EIpos): m/z=510 [M+H]⁺.

¹H-NMR (400 MHz, DMSO-D₆): δ [ppm]=1.37 (t, 3H), 2.47 (s, 3H), 3.52 (s, 3H), 3.84 (s, 3H), 4.16 (q, 2H), 6.42 (d, 1H), 6.71 (dd, 1H), 7.02 (dt, 1H), 7.13 (d, 1H), 7.20 (d, 1H), 7.37-7.43 (m, 2H), 7.90 (dd, 1H), 8.01 (d, 1H), 8.03 (d, 1H), 8.87 (d, 1H), 8.89 (d, 1H) 9.13 (sbr, 1H).

### Example 59A

### Methyl-2-bromo-5-hydroxybenzene carboxylate

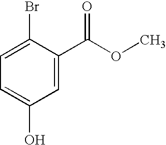

5.00 g (20.4 mmol) of methyl-2-bromo-5-methoxybenzene carboxylate was dissolved in 95 ml dichloromethane and cooled to −78° C. Then 61.0 ml (61.0 mmol) of boron tribromide, as 1N solution in dichloromethane, was slowly added dropwise. Then it was heated to room temperature and stirred overnight. Next it was submitted to solvolysis with methanol, cautiously and with ice cooling. The reaction solution was concentrated in a rotary evaporator, taken up in water and then extracted with dichloromethane (2×). The combined organic phases were dried over magnesium sulfate and the solvent was removed by distillation under reduced pressure. The raw product was purified by MPLC (Puriflash Analogix: 40M: isohexane/ethyl acetate=4/1). We obtained 3.19 g (68% of theor.) of the target compound.

LC-MS (method 4): Rₜ=0.93 min; MS (EIpos): m/z=231 [M]⁺.

¹H-NMR (400 MHz, DMSO-D₆): δ [ppm]=3.83 (s, 3H), 6.88 (dd, 1H), 7.14 (d, 1H), 7.50 (d, 1H), 10.09 (s, 1H).

### Example 60A

### Methyl-2-bromo-5-(difluoromethoxy)benzene carboxylate

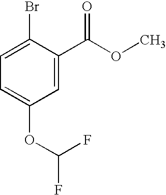

1.00 g (4.33 mmol) of the compound from example 59A was dissolved in 60 ml dimethylformamide. Then 1.65 g (10.8 mmol) difluorochlorosodium acetate and 164 mg (4.11 mmol) sodium hydroxide were added. It was stirred overnight at 100° C. and again for 24 h at 120° C. The reaction mixture was concentrated in a rotary evaporator and the residue was taken up in water. Then it was extracted with ethyl acetate (3×). The combined organic phases were then dried over magnesium sulfate and the solvent was removed by distillation under reduced pressure. The raw product was purified by MPLC (Puriflash Analogix: 40M: isohexane/ethyl acetate=95/5). We obtained 105 mg (9% of theor.) of the target compound.

LC-MS (method 4): Rₜ=1.21 min; MS (EIpos): m/z=281 [M]⁺.

¹H-NMR (400 MHz, DMSO-D₆): δ [ppm]=3.87 (s, 3H), 7.31 (t, 1H), 7.33 (dd, 1H), 7.57 (d, 1H), 7.81 (d, 1H).

### Example 61A

### Methyl-2-{[4-(quinoxalin-6-yl)-3-methyl-1-(2-methylphenyl)-1H-pyrazol-5-yl]amino}-5-(difluoromethoxy)benzene carboxylate

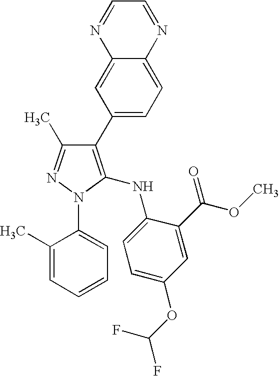

227 mg (0.534 mmol) of the compound from example 1A was dissolved in 5 ml toluene. Then it was rinsed with argon, and 4.0 mg (0.018 mmol) of palladium(II) acetate and 19.2 mg (0.036 mmol) of bis(2-diphenylphosphinophenyl)ether were added. It was stirred for 5 min at room temperature. A brown solution formed. Then 100 mg (0.356 mmol) of the compound from example 60A and 163 mg (0.498 mmol) cesium carbonate were added and the mixture was reacted overnight at 95° C. The reaction mixture was filtered on kieselguhr. It was washed again with ethyl acetate. The solvent was removed by distillation under reduced pressure. The residue was purified by preparative HPLC (eluent: acetonitrile/water, gradient 10:90→90:10). We obtained 70 mg (38% of theor.) of the target compound.

LC-MS (method 10): Rₜ=1.21 min; MS (EIpos): m/z=516 [M]⁺.

¹H-NMR (400 MHz, DMSO-D₆): δ [ppm]=2.18 (s, 3H), 2.48 (s, 3H), 3.82 (s, 3H), 6.51 (d, 1H), 6.95 (t, 1H), 7.05 (dd, 1H), 7.25 (m, 1H), 7.30-7.34 (m, 2H), 7.40 (d, 1H), 7.44 (d, 1H), 7.99 (dd, 1H), 8.05 (d, 1H), 8.13 (d, 1H), 8.89 (d, 1H), 8.90 (d, 1H), 9.16 (s, 1H).

### Example 62A

### 2,3-Difluoro-6-nitroaniline

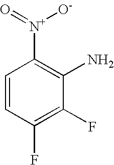

Ten batches, each of 2.00 g (11.3 mmol) of 1,2,3-trifluoro-4-nitrobenzene and 4.2 ml (29.6 mmol) of 7N methanolic ammonia solution, were reacted sequentially for 90 min at 70° C. in a single-mode microwave. The resultant solutions were combined and the volatile components were removed in a rotary evaporator. The residue was taken up in ethyl acetate, washed with water and dried over sodium sulfate. The solvent was removed by distillation at reduced pressure and the residue was recrystallized from methanol/water (1:1). We obtained 14.4 g of the target compound (73% of theor., based on the total amount of 1,2,3-trifluoro-4-nitrobenzene used).

GC-MS (method 6): Rₜ=4.09 min; MS (EIpos): m/z=174 [M]⁺.

¹H-NMR (400 MHz, DMSO-D₆): δ [ppm]=6.72 (m, 1H), 7.54 (sbr, 2H), 7.93 (m, 1H).

### Example 63A

### 3-Amino-2-fluoro-4-nitrophenol

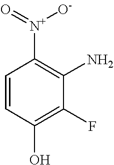

5.00 g (28.7 mmol) of example 62A was taken up in 50 ml dioxane and 6.44 g (114.9 mmol) potassium hydroxide, as solution in 20 ml water, was added. Then it was stirred overnight at the reflux temperature. The reaction mixture was added to 80 ml of 1M citric acid solution. The resultant solution was extracted with ethyl acetate (2×100 ml). The combined organic phases were washed with water and saturated aqueous sodium chloride solution and dried over magnesium sulfate. The solvent was removed and the residue was purified by MPLC (Puriflash Analogix: 40M: isohexane/ethyl acetate=3/1). We obtained 2.60 g (49% of theor.) of the target compound.

LC-MS (method 10): Rₜ=0.62 min; MS (EIneg): m/z=172 [M−H]⁻.

¹H-NMR (400 MHz, DMSO-D₆): δ [ppm]=6.30 (dd, 1H), 7.19 (sbr, 2H), 7.75 (dd, 1H), 11.09 (s, 1H).

### Example 64A

### 2-Fluoro-3-methoxy-6-nitroaniline

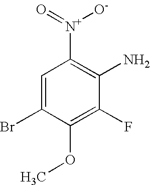

1.70 g (9.88 mmol) of example 63A was taken up in 20 ml tetrahydrofuran and 0.40 ml (9.88 mmol) methanol was added. After adding 2.85 g (10.9 mmol) triphenylphosphine it was cooled on an ice bath and 2.10 ml (10.9 mmol) diisopropylazodicarboxylate was added dropwise in the space of 5 min. The reaction mixture was stirred at room temperature for 2.5 h. The mixture was concentrated by evaporation and purified by column chromatography (silica gel: cyclohexane/ethyl acetate=3/1). We obtained 3.50 g (95% of theor.) of the target compound at 50% purity (LC-MS). The contaminant was found to be diisopropylhydrazine-1,2-dicarboxylate. The material thus obtained was used without further purification.

¹H-NMR (400 MHz, DMSO-D₆): δ [ppm]=3.92 (s, 3H), 6.61 (dd, 1H), 7.14 (sbr, 2H), 7.90 (dd, 1H).

### Example 65A

### 4-Bromo-2-fluoro-3-methoxy-6-nitroaniline

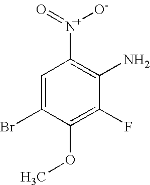

1.80 g (approx. 4.83 mmol) of example 64A was taken up in 25 ml acetic acid and 946 mg (5.32 mmol) of N-bromosuccinimide was added. It was stirred for 4 h at the reflux temperature. Then it was added to 100 ml water and the precipitated solid was separated by filtration. The solid was washed with water and was then dried under high vacuum. We obtained 1.08 g (84% of theor.) of the target compound.

¹H-NMR (400 MHz, DMSO-D₆): δ [ppm]=4.04 (d, 3H), 7.49 (sbr, 2H), 8.05 (d, 1H).

### Example 66A

### 5-Bromo-3-fluoro-4-methoxybenzene-1,2-diamine

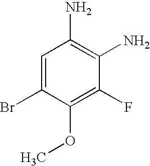

1.08 g (4.15 mmol) of example 65A was taken up in 50 ml ethanol and 10 ml water and heated to 80° C. Then 2.89 g (16.6 mmol) of sodium dithionite, as solution in 15 ml water, was added. After reaction for 60 min reaction at 80° C., a further 1.08 g (6.22 mmol) sodium dithionite was added and reaction was continued for 45 min at the same temperature. The mixture was concentrated by evaporation to the maximum possible extent and the resultant precipitate was filtered off. This was washed with water and dried under high vacuum. We obtained 600 mg (62% of theor.) of the target compound.

LC-MS (method 10): Rₜ=0.72 min; MS (EIpos): m/z=235 [M]⁺.

¹H-NMR (400 MHz, DMSO-D₆): δ [ppm]=3.67 (s, 3H), 4.64 (sbr, 2H), 4.80 (sbr, 2H), 6.50 (d, 1H).

### Example 67A

### 7-Bromo-5-fluoro-6-methoxyquinoxaline

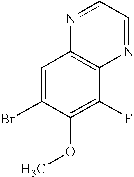

600 mg (2.55 mmol) of example 66A was dissolved in 25 ml ethanol, 307 mg (2.55 mmol) of 1,4-dioxane-2,3-diol was added and it was stirred overnight at room temperature. The mixture was concentrated by evaporation and the residue was taken up in 50 ml dichloromethane. The organic phase was washed with water and saturated aqueous sodium chloride solution. Then it was dried over magnesium sulfate and the solvent was removed in a rotary evaporator. The residue was finally dried under high vacuum. We obtained 377 mg (57% of theor.) of the target compound.

LC-MS (method 10): Rₜ=0.90 min; MS (EIpos): m/z=258 [M+H]⁺.

¹H-NMR (400 MHz, DMSO-D₆): δ [ppm]=4.12 (s, 3H), 8.32 (d, 1H), 8.97 (d, 1H), 9.01 (d, 1H).

### Example 68A

### Methyl-2-{[3-methyl-1-(2-methylphenyl)-1H-pyrazol-5-yl]amino}benzene carboxylate

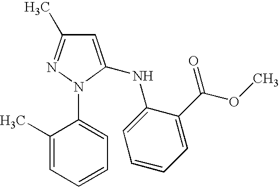

Under an argon atmosphere, 6.53 g (34.9 mmol) of 3-methyl-1-(2-methylphenyl)-1H-pyrazol-5-amine (described in WO 2004/050650, p. 30) was dissolved in 100 ml abs. toluene. It was flushed with argon, and 261 mg (1.16 mmol) of palladium(II) acetate and 1.25 g (2.33 mmol) of bis(2-diphenylphosphinophenyl)ether (DPEphos) were added. Then it was stirred for 5 min at room temperature. Then 5.00 g (23.3 mmol) of methyl-2-bromobenzoate and 5.00 g (23.3 mmol) cesium carbonate were added and then stirred overnight at 95° C. The reaction mixture was cooled and filtered on kieselguhr. It was washed again with ethyl acetate. Then the combined organic phases were concentrated in a rotary evaporator and the residue was purified by column chromatography (silica gel: cyclohexane/ethyl acetate=7/3). We obtained 7.37 g (98% of theor.) of the target compound.

LC-MS (method 4): Rₜ=1.34 min; MS (EIpos): m/z=322 [M+H]⁺.

¹H-NMR (400 MHz, DMSO-D₆): δ [ppm]=2.03 (s, 3H), 2.24 (s, 3H), 3.72 (s, 3H), 6.21 (s, 1H), 6.83 (t, 1H), 7.24 (d, 1H), 7.29-7.34 (m, 2H), 7.36-7.41 (m, 2H), 7.49 (mc, 1H), 7.83 (dd, 1H), 9.27 (s, 1H).

### Example 69A

### Methyl-2-{[4-bromo-3-methyl-1-(2-methylphenyl)-1H-pyrazol-5-yl]amino}benzene carboxylate

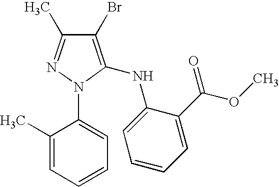

7.35 g (22.9 mmol) of the compound from example 68A was dissolved in 150 ml dichloromethane and, while cooling with ice, 3.27 g (11.5 mmol) of 1,3-dibromo-5,5-dimethylhydantoin was added. After reaction for 10 minutes, it was with diluted with 150 ml dichloromethane and washed with 150 ml each of water, saturated aqueous sodium hydrogen carbonate solution and with 10% aqueous sodium thiosulfate solution. Then it was dried over magnesium sulfate and the solvent was removed in a rotary evaporator. We obtained 8.28 g (90% of theor.) of the target compound.

LC-MS (method 10): Rₜ=1.33 min; MS (EIpos): m/z=400 [M]⁺.

¹H-NMR (400 MHz, DMSO-D₆): δ [ppm]=2.09 (s, 3H), 2.27 (s, 3H), 3.80 (s, 3H), 6.55 (d, 1H), 6.79 (t, 1H), 7.23 (m, 1H), 7.28-7.36 (m, 3H), 7.39 (t, 1H), 7.79 (dd, 1H), 8.99 (s, 1H).

### Example 70A

### Methyl-2-{[4-(8-fluoro-7-methoxyquinoxalin-6-yl)-3-methyl-1-(2-methylphenyl)-1H-pyrazol-5-yl]amino}benzene carboxylate

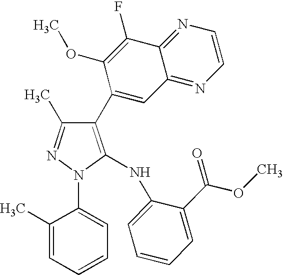

### Stage 1

Under an argon atmosphere, 375 mg (1.46 mmol) of example 67A was taken up in 15 ml dioxane. Then 258 mg (2.63 mmol) potassium acetate, 95 mg (0.12 mmol) of 1,1′-bis-(diphenylphosphino)ferrocene palladium(II) chloride-dichloromethane complex and 407 mg (1.61 mmol) of 4,4,4′4′5,5,5′5′-octamethyl-2,2′-bi-1,3,2-dioxaborolan were added. The reaction mixture was stirred overnight at 130° C. oil bath temperature. After cooling, dioxane was added to the reaction mixture and it was filtered on kieselguhr. It was washed again with ethyl acetate. The filtrate was concentrated in a rotary evaporator at reduced pressure and dried under high vacuum. We obtained 610 mg of 5-fluoro-6-methoxy-7-(4,4,5,5-tetramethyl-1,3,2-dioxaborolan-2-yl)quinoxaline as raw product. This was reacted subsequently without further purification.

### Stage 2

Under an argon atmosphere, 209 mg of the 5-fluoro-6-methoxy-7-(4,4,5,5-tetramethyl-1,3,2-dioxaborolan-2-yl)quinoxaline raw product thus obtained, together with 300 mg (0.813 mmol) of example 69A, 25 mg (0.090 mmol) of tricyclohexylphosphine, 34 mg (0.037 mmol) of tris(dibenzylidene-acetone)-dipalladium and 1.27 ml (1.27 mmol) of 1M potassium phosphate solution, was taken up in 5 ml dioxane. Then it was reacted overnight at 100° C. After cooling, the mixture was filtered on kieselguhr, washed again with ethyl acetate and the volatile components were removed in a rotary evaporator. The residue was purified by preparative HPLC (eluent: acetonitrile/water, gradient 10:90→90:10). We obtained 161 mg (43% of theor.) of the target compound at approx. 50% purity (LC-MS). The contaminant was identified as methyl-2-{[3-methyl-1-(2-methylphenyl)-1H-pyrazol-5-yl]amino}benzene carboxylate. The material thus obtained was used subsequently without further processing.

LC-MS (method 10): Rₜ=1.23 min; MS (EIpos): m/z=498 [M+H]⁺.

### Example 71A

### Methyl-2-[(3-methyl-1-phenyl-1H-pyrazol-5-yl)amino]benzene carboxylate

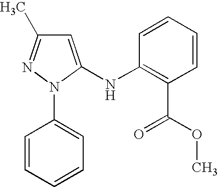

Under an argon atmosphere, 8.00 g (46.184 mmol) of 5-amino-3-methyl-1-phenylpyrazole was dissolved in 130 ml toluene and 0.346 g (1.539 mmol) of palladium(II) acetate and 1.658 g (3.079 mmol) bis[2-diphenylphosphino)-phenyl]-ether were added. The mixture was stirred for 5 min at room temperature. Then 6.621 g (30.789 mmol) of methyl-2-bromobenzoate and 14.045 g (43.105 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by preparative HPLC (eluent: acetonitrile/water, gradient 20:80→90:10). We obtained 9.36 g (66% of theor.) of the target compound.

LC-MS (method 10): Rₜ=1.18 min; MS (EIpos): m/z=308 [M+H]⁺.

¹H-NMR (400 MHz, DMSO-D₆): δ [ppm]=2.25 (s, 3H), 3.80 (s, 3H), 6.23 (s, 1H), 6.82 (t, 1H), 6.94 (d, 1H), 7.33 (t, 1H), 7.37-7.49 (m, 3H), 7.49-7.58 (m, 2H), 7.86 (d, 1H), 9.38 (s, 1H).

### Example 72A

### Methyl-2-[(4-bromo-3-methyl-1-phenyl-1H-pyrazol-5-yl)amino]benzene carboxylate

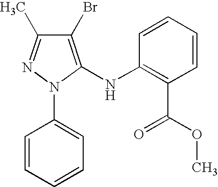

9.370 g (30.486 mmol) of the compound from example 71A was dissolved in 160 ml dichloromethane and, while cooling with ice, 4.358 g (15.243 mmol) of 1,3-dibromo-5,5-dimethylhydantoin was added in portions. Reaction testing by HPLC showed that reaction was completed after 20 min. Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 11.441 g (96% of theor.) of the target compound.

LC-MS (method 10): Rₜ=1.32 min; MS (EIpos): m/z=386 [M+H]⁺.

¹H-NMR (400 MHz, DMSO-D₆): δ [ppm]=2.28 (s, 3H), 3.86 (s, 3H), 6.36 (d, 1H), 6.79 (t, 1H), 7.26-7.37 (m, 2H), 7.37-7.46 (m, 2H), 7.50-7.60 (m, 2H), 7.84 (d, 1H), 9.14 (s, 1H).

### Example 73A

### Methyl-2-{[4-(8-fluoro-7-methoxyquinoxalin-6-yl)-3-methyl-1-phenyl-1H-pyrazol-5-yl]amino}benzene carboxylate

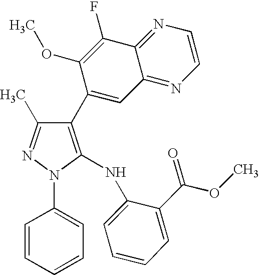

Under an argon atmosphere, 215 mg of 5-fluoro-6-methoxy-7-(4,4,5,5-tetramethyl-1,3,2-dioxaborolan-2-yl)quinoxaline (raw product, see example 70A Stage 1), together with 300 mg (0.777 mmol) of example 72A, 26 mg (0.093 mmol) of tricyclohexylphosphine, 36 mg (0.039 mmol) of tris(dibenzylidene-acetone)-dipalladium and 1.32 ml (1.32 mmol) of 1M potassium phosphate solution, was taken up in 5 ml dioxane. Then it was reacted overnight at 100° C. After cooling, the mixture was filtered on kieselguhr, washed again with ethyl acetate and the volatile components were removed in a rotary evaporator. The residue was purified by preparative HPLC (eluent: acetonitrile/water, gradient 10:90→90:10). We obtained 162 mg (43% of theor.) of the target compound at approx. 60% purity (LC-MS). The contaminant was identified as methyl-2-{[3-methyl-1-(2-methylphenyl)-1H-pyrazol-5-yl]amino}benzene carboxylate. The material thus obtained was used subsequently without further processing.

LC-MS (method 10): Rₜ=1.23 min; MS (EIpos): m/z=483 [M+H]⁺.

### Example 74A

### 7-Bromo-2-methoxyquinoxaline

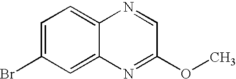

600 mg (2.67 mmol) of 7-bromoquinoxalin-2(1H)-one [Lumma et al., J. Med. Chem. 1981, 24, 93] and 1.73 ml (3.47 mmol) trimethylsilyldiazomethane were taken up in 5.25 ml methanol/acetonitrile/dichloromethane (1/10/10). Then 0.483 ml (3.47 mmol) triethylamine was added and it was stirred overnight at room temperature. The mixture was concentrated by evaporation and the residue was purified by MPLC (Puriflash Analogix: 40M: isohexane/ethyl acetate=9/1→isohexane/ethyl acetate=3/1). We obtained 140 mg (22% of theor.) of the target compound.

LC-MS (method 10): Rₜ=1.09 min; MS (EIpos): m/z=240 [M+H]⁺.

¹H-NMR (400 MHz, DMSO-D₆): δ [ppm]=4.04 (s, 3H), 7.78 (dd, 1H), 7.96 (d, 1H), 8.06 (d, 1H), 8.64 (s, 1H).

### Example 75A

### Methyl-2-{[4-(3-methoxyquinoxalin-6-yl)-3-methyl-1-(2-methylphenyl)-1H-pyrazol-5-yl]amino}benzene carboxylate

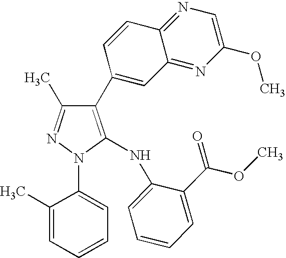

### Stage 1

Under an argon atmosphere, 140 mg (0.586 mmol) of example 74A was dissolved in 15 ml dioxane. Then 172 mg (1.76 mmol) potassium acetate, 38 mg (0.047 mmol) of 1,1′-bis-(diphenylphosphino)ferrocene palladium(II) chloride-dichloromethane complex and 163 mg (0.644 mmol) of 4,4,4′4′5,5,5′5′-octamethyl-2,2′-bi-1,3,2-dioxaborolan were added. The reaction mixture was stirred overnight at 130° C. oil bath temperature. After cooling, dioxane was added to the reaction mixture and it was filtered on kieselguhr. It was washed again with ethyl acetate. The filtrate was concentrated in a rotary evaporator at reduced pressure and dried under high vacuum. We obtained 152 mg of 2-methoxy-7-(4,4,5,5-tetramethyl-1,3,2-dioxaborolan-2-yl)quinoxaline as raw product. This was reacted subsequently without further purification.

### Stage 2

Under an argon atmosphere, 150 mg of the 2-methoxy-7-(4,4,5,5-tetramethyl-1,3,2-dioxaborolan-2-yl)quinoxaline raw product thus obtained, together with 142 mg (0.356 mmol) of example 69A, 12 mg (0.043 mmol) of tricyclohexylphosphine, 16 mg (0.018 mmol) of tris(dibenzylidene-acetone)-dipalladium and 0.61 ml (0.61 mmol) of 1M potassium phosphate solution was taken up in 10 ml dioxane. Then it was reacted overnight at 100° C. After cooling, the mixture was filtered on kieselguhr, washed again with ethyl acetate and the volatile components were removed in a rotary evaporator. The mixture was concentrated by evaporation and the residue was purified by MPLC (Puriflash Analogix: 40S: isohexane/ethyl acetate=95/5→isohexane/ethyl acetate=10/90). We obtained 30 mg (approx. 14% of theor.) of the target compound at approx. 80% purity (LC-MS). The material thus obtained was used subsequently without further processing.

LC-MS (method 10): Rₜ=1.34 min; MS (EIpos): m/z=480 [M+H]⁺.

### Example 76A

### Methyl-2-{[4-(quinolin-7-yl)-3-methyl-1-(2-methylphenyl)-1H-pyrazol-5-yl]amino}benzene carboxylate

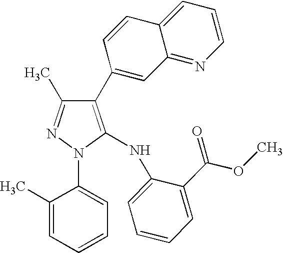

### Stage 1

Under an argon atmosphere, 600 mg (2.884 mmol) of 7-bromoquinoline [Butler et al., J. Heterocycl. Chem. 1975, 12, 1015] was dissolved in 25 ml dioxane. Then 509 mg (5.19 mmol) potassium acetate, 188 mg (0.231 mmol) of 1,1′-bis-(diphenylphosphino)ferrocene palladium(II) chloride-dichloromethane complex and 805 mg (3.17 mmol) of 4,4,4′4′5,5,5′5′-octamethyl-2,2′-bi-1,3,2-dioxaborolan were added. The reaction mixture was stirred overnight at 130° C. oil bath temperature. After cooling, dioxane was added to the reaction mixture and it was filtered on kieselguhr. It was washed again with ethyl acetate. The filtrate was concentrated in a rotary evaporator at reduced pressure and dried under high vacuum. We obtained 1100 mg of 7-(4,4,5,5-tetramethyl-1,3,2-dioxaborolan-2-yl)quinoline as raw product. This was reacted subsequently without further purification.

### Stage 2

Under an argon atmosphere, 202 mg of the 7-(4,4,5,5-tetramethyl-1,3,2-dioxaborolan-2-yl)quinoline raw product thus obtained, together with 150 mg (0.468 mmol) of example 69A, 13 mg (0.045 mmol) of tricyclohexylphosphine, 19 mg (0.019 mmol) of tris(dibenzylidene-acetone)-dipalladium and 0.637 ml (0.637 mmol) of 1M potassium phosphate solution, was taken up in 5 ml dioxane. Then it was reacted overnight at 100° C. The solid constituents were separated by filtration, the solvent was removed in a rotary evaporator and the reaction mixture was purified by HPLC (Puriflash Analogix: 40S: isohexane/ethyl acetate=95/5→isohexane/ethyl acetate=10/90). We obtained 37 mg (22% of theor.) of the target compound.

LC-MS (method 4): Rₜ=1.27 min; MS (EIpos): m/z=449 [M+H]⁺.

¹H-NMR (400 MHz, DMSO-D₆): δ [ppm]=2.18 (s, 3H), 2.47 (s, 3H), 3.79 (s, 3H), 6.48 (d, 1H), 6.59 (t, 1H), 7.16 (t, 1H), 7.23 (m, 1H), 7.27-7.36 (m, 2H), 7.43 (d, 1H), 7.47 (dd, 1H), 7.65 (dd, 1H), 7.71 (dd, 1H), 7.92 (d, 1H), 8.04 (s, 1H), 8.29 (d, 1H), 8.85 (d, 1H), 9.21 (s, 1H).

### Example 77A

### 7-Bromo-N,N-dimethylquinoxalin-2-amine

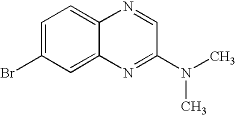

500 mg (2.053 mmol) of 2-chloro-7-bromoquinoline [Wolf et al., J. Am. Chem. Soc., 1949, 71, 6] and 5.13 ml (10.27 mmol) of dimethylamine were reacted in a single mode microwave for 8 h at 120° C. Then the volatile components were removed in a rotary evaporator and the residue was purified by HPLC (Puriflash Analogix: 40S: isohexane/ethyl acetate=95/5→isohexane/ethyl acetate=10/90). We obtained 230 mg (44% of theor.) of the target compound.

LC-MS (method 10): Rₜ=1.05 min; MS (EIpos): m/z=252 [M]⁺.

¹H-NMR (400 MHz, DMSO-D₆): δ [ppm]=3.23 (s, 6H), 7.47 (dd, 1H), 7.70-7.79 (m, 2H), 8.71 (s, 1H).

### Example 78A

### Methyl-2-({4-[3-(dimethylamino)quinoxalin-6-yl]-3-methyl-1-(2-methylphenyl)-1H-pyrazol-5-yl}amino)benzene carboxylate

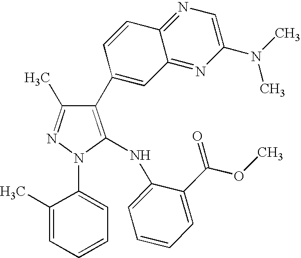

### Stage 1

Under an argon atmosphere, 230 mg (0.912 mmol) of example 77A was dissolved in 10 ml dioxane. Then 269 mg (2.74 mmol) potassium acetate, 59 mg (0.073 mmol) of 1,1′-bis-(diphenylphosphino)ferrocene palladium(II) chloride-dichloromethane complex and 255 mg (1.003 mmol) of 4,4,4′4′5,5,5′5′-octamethyl-2,2′-bi-1,3,2-dioxaborolan were added. The reaction mixture was stirred overnight at 130° C. oil bath temperature. After cooling, dichloromethane was added to the reaction mixture and it was filtered on kieselguhr. It was washed again with ethyl acetate. The filtrate was concentrated in a rotary evaporator at reduced pressure and dried under high vacuum. We obtained 280 mg of N,N-dimethyl-7-(4,4,5,5-tetramethyl-1,3,2-dioxaborolan-2-yl)quinoxalin-2-amine [42%, also contains 45% of the corresponding boric acid (LC-MS)] as raw product. This was reacted subsequently without further purification.

### Stage 2

Under an argon atmosphere, 272 mg of the N,N-dimethyl-7-(4,4,5,5-tetramethyl-1,3,2-dioxaborolan-2-yl)quinoxalin-2-amine raw product thus obtained, together with 242 mg (0.606 mmol) of example 69A, 20 mg (0.073 mmol) of tricyclohexylphosphine, 28 mg (0.030 mmol) of tris(dibenzylidene-acetone)-dipalladium and 1.03 ml (1.03 mmol) of 1M potassium phosphate solution, was taken up in 17 ml dioxane. Then it was reacted overnight at 100° C. After cooling, the mixture was filtered on kieselguhr, washed again with ethyl acetate and the volatile components were removed in a rotary evaporator. The mixture was concentrated by evaporation and the residue was purified by MPLC (Puriflash Analogix: 40S: isohexane/ethyl acetate=95/5→isohexane/ethyl acetate=10/90). We obtained 79 mg (26% of theor.) of the target compound.

LC-MS (method 10): Rₜ=1.27 min; MS (EIpos): m/z=493 [M+H]⁺.

¹H-NMR (400 MHz, DMSO-D₆): δ [ppm]=2.17 (s, 3H), 2.41 (s, 3H), 3.20 (s, 6H), 3.79 (s, 3H), 6.46 (d, 1H), 6.59 (t, 1H), 7.15 (t, 1H), 7.23 (mc, 1H), 7.27-7.35 (m, 2H), 7.41 (t, 2H), 7.60-7.67 (m, 2H), 7.71 (d, 1H), 8.61 (s, 1H), 9.17 (s, 1H).

### Example 79A

### 3-Amino-6-bromo-2-fluoro-4-nitrophenol

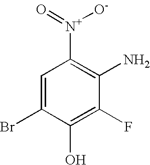

2.60 g (15.1 mmol) of example 63A was taken up in 80 ml acetic acid and 2.96 g (16.6 mmol) of N-bromosuccinimide was added. Then it was stirred for 4 h at the reflux temperature. The solvent was removed and the residue was purified by column chromatography (silica gel: cyclohexane/ethyl acetate=1/1). We obtained 2.10 g (46% of theor.) of the target compound.

LC-MS (method 10): Rₜ=0.81 min; MS (EIpos): m/z=251 [M]⁺.

¹H-NMR (400 MHz, DMSO-D₆): δ [ppm]=7.34 (sbr, 2H), 8.01 (d, 1H), 11.84 (sbr, 1H).

### Example 80A

### 3,4-Diamino-6-bromo-2-fluorophenol

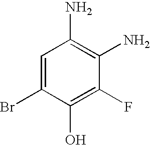

2.10 g (8.37 mmol) of example 79A was taken up in 50 ml ethanol and 7.56 g (33.5 mmol) of tin(II) chloride dihydrate was added. Then it was stirred overnight at 70° C. The mixture was concentrated by evaporation and the residue was taken up in 100 ml water. Then it was made weakly alkaline with saturated aqueous sodium hydrogen carbonate solution and 100 ml ethyl acetate was added. It was filtered on kieselguhr and the filter cake was washed again with ethyl acetate. The organic phase was separated and the aqueous phase was extracted with ethyl acetate (2×). The combined organic phases were dried and the solvent was removed in a rotary evaporator. In this way we obtained 1.10 g (58% of theor.) of the target compound at a purity of 97% (LC-MS).

LC-MS (method 4): Rₜ=0.26 min; MS (EIpos): m/z=221 [M]⁺.

### Example 81A

### 7-Bromo-5-fluoroquinoxalin-6-ol

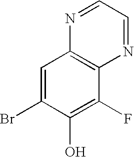

1.10 g (4.98 mmol) of example 80A was taken up in 40 ml ethanol and 598 mg (4.98 mmol) of 1,4-dioxane-2,3-diol was added. Then it was stirred overnight at room temperature. The volatile components were removed and the residue was taken up in dichloromethane. It was washed with water and saturated aqueous sodium chloride solution. Then it was dried over magnesium sulfate. The mixture was concentrated by evaporation and the residue was purified by MPLC (Puriflash Analogix: 40M: isohexane/ethyl acetate=1/1). We obtained 603 mg (49% of theor.) of the target compound.

LC-MS (method 10): Rₜ=0.68 min; MS (EIpos): m/z=243 [M]⁺.

¹H-NMR (400 MHz, DMSO-D₆): δ [ppm]=8.24 (d, 1H), 8.85 (d, 1H), 8.93 (d, 1H), 11.61 (s, 1H).

### Example 82A

### Methyl-2-{[4-(8-fluoro-7-hydroxyquinoxalin-6-yl)-3-methyl-1-(2-methylphenyl)-1H-pyrazol-5-yl]amino}benzene carboxylate

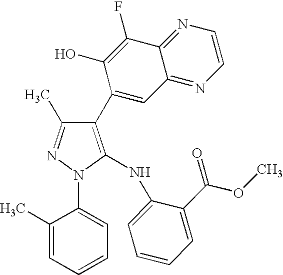

### Stage 1

Under an argon atmosphere, 230 mg (0.946 mmol) of example 81A was taken up in 10 ml dioxane. Then 168 mg (1.70 mmol) potassium acetate, 62 mg (0.076 mmol) of 1,1′-bis-(diphenylphosphino)ferrocene palladium(II) chloride-dichloromethane complex and 264 mg (1.04 mmol) of 4,4,4′4′5,5,5′5′-octamethyl-2,2′-bi-1,3,2-dioxaborolan were added. The reaction mixture was stirred overnight at 130° C. oil bath temperature. After cooling, dioxane was added to the reaction mixture and it was filtered on kieselguhr. It was washed again with ethyl acetate. The filtrate was concentrated in a rotary evaporator at reduced pressure and dried under high vacuum. We obtained 474 mg of 8-fluoro-7-hydroxyquinoxalin-6-yl)boric acid as raw product at 32% purity (LC-MS). This was reacted subsequently without further purification.

### Stage 2

Under an argon atmosphere, 202 mg of the 8-fluoro-7-hydroxyquinoxalin-6-yl)boric acid raw product thus obtained, together with 100 mg (0.813 mmol) of example 69A, 8.4 mg (0.030 mmol) of tricyclohexylphosphine, 11.4 mg (0.012 mmol) of tris(dibenzylidene-acetone)-dipalladium and 0.425 ml (0.425 mmol) of 1M potassium phosphate solution, was taken up in 5 ml dioxane. Then it was reacted overnight at 100° C. After cooling, the mixture was filtered on kieselguhr, washed again with ethyl acetate and the volatile components were removed in a rotary evaporator. The residue was purified by preparative HPLC (eluent: acetonitrile/water, gradient 10:90→90:10). We obtained 5 mg (4% of theor.) of the target compound.

LC-MS (method 10): Rₜ=1.05 min; MS (EIpos): m/z=484 [M+H]⁺.

### Example 83A

### 7-Bromo-6-ethoxy-5-fluoroquinoxaline

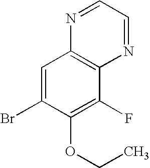

150 mg (0.617 mmol) of example 81A was taken up in 5 ml tetrahydrofuran and 0.036 ml (0.617 mmol) ethanol was added. After adding 178 mg (0.679 mmol) triphenylphosphine, it was cooled on an ice bath and 0.131 ml (0.679 mmol) diisopropylazodicarboxylate was added dropwise in the space of 5 min. The reaction mixture was heated slowly to room temperature and then stirred for a further 30 min at ambient temperature. The volatile components were removed in a rotary evaporator and the residue was then purified by preparative HPLC (eluent: acetonitrile/water, gradient 10:90→90:10). We obtained 100 mg (60% of theor.) of the target compound.

LC-MS (method 2): Rₜ=2.07 min; MS (EIpos): m/z=271 [M]⁺.

¹H-NMR (400 MHz, DMSO-D₆): δ [ppm]=1.43 (t, 3H), 4.37 (q, 2H), 8.32 (d, 1H), 8.98 (d, 1H), 9.01 (d, 1H).

### Example 84A

### Methyl-2-{[4-(7-ethoxy-8-fluoroquinoxalin-6-yl)-3-methyl-1-(2-methylphenyl)-1H-pyrazol-5-yl]amino}benzene carboxylate

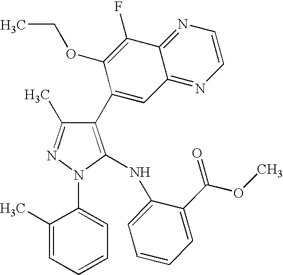

### Stage 1

Under an argon atmosphere, 100 mg (0.369 mmol) of example 83A was dissolved in 4 ml dioxane. Then 65.1 mg (0.664 mmol) potassium acetate, 24.1 mg (0.030 mmol) of 1,1′-bis-(diphenylphosphino)ferrocene palladium(II) chloride-dichloromethane complex and 103 mg (0.406 mmol) of 4,4,4′4′5,5,5′5′-octamethyl-2,2′-bi-1,3,2-dioxaborolan were added. The reaction mixture was stirred overnight at 130° C. oil bath temperature. After cooling, dichloromethane was added to the reaction mixture and it was filtered on kieselguhr. It was washed again with ethyl acetate. The filtrate was concentrated in a rotary evaporator at reduced pressure and dried under high vacuum. We obtained 200 mg of 6-ethoxy-5-fluoro-7-(4,4,5,5-tetramethyl-1,3,2-dioxaborolan-2-yl)quinoxaline [87%, (LC-MS)] as raw product. This was reacted subsequently without further purification.

### Stage 2

Under an argon atmosphere, 100 mg of the 6-ethoxy-5-fluoro-7-(4,4,5,5-tetramethyl-1,3,2-dioxaborolan-2-yl)quinoxaline raw product thus obtained, together with 73 mg (0.312 mmol) of example 69A, 8.4 mg (0.030 mmol) of tricyclohexylphosphine, 11.4 mg (0.012 mmol) of tris(dibenzylidene-acetone)-dipalladium and 0.425 ml (0.425 mmol) of 1M potassium phosphate solution, was taken up in 4 ml dioxane. Then it was reacted overnight at the reflux temperature. The volatile components were removed in a rotary evaporator and the residue was then purified by preparative HPLC (eluent: acetonitrile/water, gradient 10:90→90:10). We obtained 37 mg (28% of theor.) of the target compound.

LC-MS (method 10): Rₜ=1.26 min; MS (EIpos): m/z=512 [M+H]⁺.

¹H-NMR (400 MHz, DMSO-D₆): δ [ppm]=1.30 (t, 3H), 2.21 (s, 3H), 2.31 (s, 3H), 3.78 (s, 3H), 4.20 (q, 2H), 6.48 (d, 1H), 6.56 (t, 1H), 7.12 (t, 1H), 7.16-7.35 (m, 3H), 7.39 (d, 1H), 7.59 (d, 1H), 7.93 (s, 1H), 8.91 (d, 2H), 9.15 (s, 1H).

### Example 85A

### 2-[(7-Bromo-5-fluoroquinoxalin-6-yl)oxy]ethyl acetate

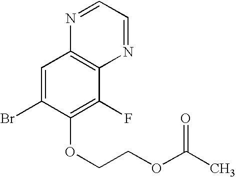

150 mg (0.617 mmol) of example 81A was taken up in 5 ml tetrahydrofuran and 0.128 ml (0.617 mmol, 50%) 2-acetoxyethanol was added. After adding 178 mg (0.679 mmol) triphenylphosphine it was cooled on an ice bath and 0.131 ml (0.679 mmol) diisopropylazodicarboxylate was added dropwise in the space of 5 min. The reaction mixture was heated slowly to room temperature and then stirred for a further 30 min at room temperature. The volatile components were removed in a rotary evaporator and the residue was then purified by preparative HPLC (eluent: acetonitrile/water, gradient 10:90→90:10). We obtained 230 mg of the target compound at 30% purity (LC-MS). Triphenylphosphine oxide was identified as the contaminant. The material thus obtained was reacted without further purification steps.

LC-MS (method 10): Rₜ=0.90 min; MS (EIpos): m/z=329 [M]⁺.

### Example 86A

### Methyl-2-{[4-{7-[2-(acetyloxy)ethoxy]-8-fluoroquinoxalin-6-yl}-3-methyl-142-methylphenyl)-1H-pyrazol-5-yl]amino}benzene carboxylate

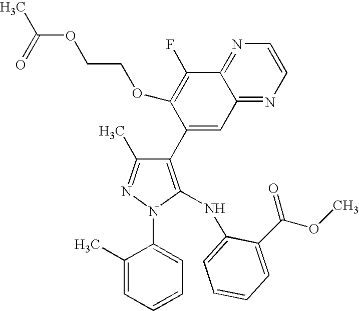

### Stage 1

Under an argon atmosphere, 164 mg of example 85A was dissolved in 5 ml dioxane. Then 88.3 mg (0.900 mmol) potassium acetate, 32.7 mg (0.040 mmol) of 1,1′-bis-(diphenylphosphino)ferrocene palladium(II) chloride-dichloromethane complex and 139 mg (0.550 mmol) of 4,4,4′4′5,5,5′5′-octamethyl-2,2′-bi-1,3,2-dioxaborolan were added. The reaction mixture was stirred overnight at 110° C. oil bath temperature. After cooling, dichloromethane was added to the reaction mixture and it was filtered on kieselguhr. It was washed again with ethyl acetate. The filtrate was concentrated in a rotary evaporator at reduced pressure and dried under high vacuum. We obtained 400 mg of {7-[2-(acetyloxy)ethoxy]-8-fluoroquinoxalin-6-yl}boric acid [17%, (LC-MS)] as raw product. This was reacted subsequently without further purification.

### Stage 2

Under an argon atmosphere, 100 mg of the {7-[2-(acetyloxy)ethoxy]-8-fluoroquinoxalin-6-yl}boric acid raw product thus obtained, together with 73 mg (0.312 mmol) of example 69A, 8.4 mg (0.030 mmol) of tricyclohexylphosphine, 11.4 mg (0.012 mmol) of tris(dibenzylidene-acetone)-dipalladium and 0.425 ml (0.425 mmol) of 1M potassium phosphate solution, was taken up in 4 ml dioxane. Then it was reacted overnight at the reflux temperature. The volatile components were removed in a rotary evaporator and the residue was then purified by preparative HPLC (eluent: acetonitrile/water, gradient 10:9090:10). We obtained 40 mg (23% of theor., relative to example 69A) of the target compound.

LC-MS (method 1): Rₜ=2.56 min; MS (EIpos): m/z=570 [M+H]⁺.

### Example 87A

### 6-Bromo-4-methoxyquinoline

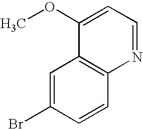

300 mg (1.24 mmol) of 6-bromo-4-chloroquinoline [Lin et al., J. Med. Chem. 1978, 21, 268] was taken up in 4 ml methanol and 1.15 ml (6.19 mmol) methanolic sodium methylate solution (30 wt. %) was added. Then it was reacted in a single mode microwave for 1 h at 120° C. The solvent was removed in a rotary evaporator and the residue was partitioned between water and ethyl acetate. The aqueous phase was extracted with ethyl acetate and the combined organic phases were dried over magnesium sulfate. The solvent was removed by distillation at reduced pressure. In this way we obtained 150 mg (36% of theor.) of the target compound.

LC-MS (method 2): Rₜ=1.24 min; MS (EIpos): m/z=238 [M]⁺.

### Example 88A

### Methyl-2-{[4-(4-methoxyquinolin-6-yl)-3-methyl-1-(2-methylphenyl)-1H-pyrazol-5-yl]amino}benzene carboxylate

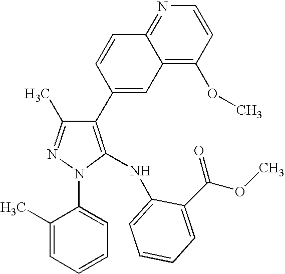

### Stage 1

Under an argon atmosphere, 150 mg of example 87A was dissolved in 4 ml dioxane. Then 111.3 mg (1.134 mmol) potassium acetate, 41.2 mg (0.050 mmol) of 1,1′-bis-(diphenylphosphino)-ferrocene palladium(II) chloride-dichloromethane complex and 176 mg (0.693 mmol) of 4,4,4′4′5,5,5′5′-octamethyl-2,2′-bi-1,3,2-dioxaborolan were added. The reaction mixture was stirred overnight at 110° C. oil bath temperature. After cooling, dichloromethane was added to the reaction mixture and it was filtered on kieselguhr. It was washed again with ethyl acetate. The filtrate was concentrated in a rotary evaporator at reduced pressure and dried under high vacuum. We obtained 227 mg of 4-methoxy-6-(4,4,5,5-tetramethyl-1,3,2-dioxaborolan-2-yl)quinoline [47%, (LC-MS)] as raw product. This was reacted subsequently without further purification.

### Stage 2

Under an argon atmosphere, 134 mg of the 4-methoxy-6-(4,4,5,5-tetramethyl-1,3,2-dioxaborolan-2-yl)quinoline raw product thus obtained, together with 150 mg (0.375 mmol) of example 69A, 12.6 mg (0.045 mmol) of tricyclohexylphosphine, 17.2 mg (0.019 mmol) of tris(dibenzylidene-acetone)-dipalladium and 0.637 ml (0.637 mmol) of 1M potassium phosphate solution, was taken up in 4 ml dioxane. Then it was reacted overnight at the reflux temperature. After filtration, the volatile components were removed in a rotary evaporator. The residue was then purified by preparative HPLC (eluent: acetonitrile/water, gradient 10:90→90:10). We obtained 99 mg (54% of theor., relative to example 69A) of the target compound.

LC-MS (method 10): Rₜ=0.96 min; MS (EIpos): m/z=479 [M+H]⁺.

¹H-NMR (400 MHz, DMSO-D₆): δ [ppm]=2.18 (s, 3H), 2.44 (s, 3H), 3.80 (s, 3H), 3.97 (s, 3H), 6.46 (d, 1H), 6.59 (t, 1H), 6.97 (d, 1H), 7.15 (mc, 1H), 7.25 (mc, 1H), 7.28-7.35 (m, 2H), 7.44 (d, 1H), 7.67 (dd, 1H), 7.82 (dd, 1H), 7.87 (d, 1H), 8.11 (d, 1H), 8.66 (d, 1H), 9.18 (s, 1H).

### Example 89A

### 5-Bromo-3-fluorobenzene-1,2-diamine

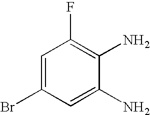

5.00 g (21.275 mmol) of 4-bromo-2fluoro-6-nitroaniline was dissolved in 190 ml ethanol, 19.20 g (85.100 mmol) of tin(II) chloride dihydrate was added and it was stirred overnight at 70° C. After cooling, the mixture was concentrated in a rotary evaporator, water was added and it was made weakly alkaline with saturated aqueous sodium hydrogen carbonate solution. The mixture was extracted three times with ethyl acetate, the combined organic phases were dried over sodium sulfate and concentrated in a rotary evaporator. The residue was dried under high vacuum. We obtained 4.18 g (92% of theor.) of the target compound, which was reacted further without further purification.

LC-MS (method 3): Rₜ=1.29 min; MS (EIpos): m/z=205 [M+H]⁺.

¹H-NMR (400 MHz, DMSO-D₆): δ [ppm]=4.52 (sbr, 2H), 5.10 (sbr, 2H), 6.49-6.52 (m, 2H).

### Example 90A

### 7-Bromo-5-fluoroquinoxaline

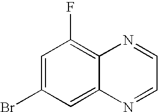

4.00 g (19.509 mmol) of the compound from example 89A was dissolved in 100 ml ethanol and 2.42 g (19.509 mmol) of 2,3-dihydroxy-1,4-dioxane was added. The mixture was stirred for 4 h at room temperature and a further 2.42 g (19.509 mmol) of 2,3-dihydroxy-1,4-dioxane was added. After stirring for 24 h at room temperature, the mixture was concentrated in a rotary evaporator and the residue was purified by silica-gel chromatography (eluent: dichloromethane/methanol=30:1). We obtained 3.60 g (80% of theor.) of the target compound.

LC-MS (method 1): Rₜ=1.79 min; MS (EIpos): m/z=227 [M+H]⁺.

¹H-NMR (400 MHz, DMSO-D₆): δ [ppm]=8.06 (dd, 1H), 8.24 (t, 1H), 9.05 (d, 1H), 9.07 (d, 1H).

### Example 91A

### 5-Fluoro-7-(4,4,5,5-tetramethyl-1,3,2-dioxaborolan-2-yl)quinoxaline

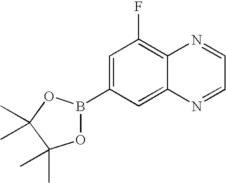

Under an argon atmosphere, 750 mg (0.819 mmol) of tris-(dibenzylidene-acetone)-dipalladium(0) and 551 mg (1.966 mmol) of tricyclohexylphosphine were dissolved in 80 ml dioxane. 3814 mg (15.019 mmol) of 4,4,4′4′5,5,5′5′-octamethyl-2,2′-bi-1,3,2-dioxaborolan, 3100 mg (13.654 mmol) of the compound from example 90A and 2010 mg (20.4813 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 7.08 g of the raw product (purity 69% according to GC-MS), which was reacted further without further purification.

GC-MS (method 6): Rₜ=6.78 min; MS (EIpos): m/z=274 [M]⁺.

### Example 92A

### Methyl-2-{[4-(8-fluoroquinoxalin-6-yl)-3-methyl-1-(2-methylphenyl)-1H-pyrazol-5-yl]amino}benzene carboxylate

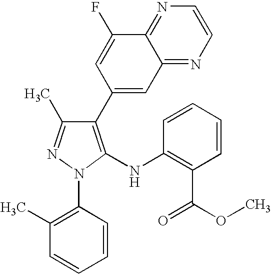

69 mg (0.075 mmol) of tris-(dibenzylidene-acetone)-dipalladium(0) and 50 mg (0.180 mmol) of tricyclohexylphosphine were added to 2.054 g of the raw product from example 91A. It was evacuated 5 times and was ventilated with argon in each case. Then 4 ml dioxane, 0.600 g (1.499 mmol) of the compound from example 69A and 2 ml of 1.27 M aqueous potassium phosphate solution were added. It was stirred for 2 h at 100° C. After cooling, dioxane was added and it was filtered on Celite. The filtrate was concentrated by evaporation and purified by preparative HPLC (eluent: acetonitrile/water, gradient 10:90→90:10). We obtained 661 mg (94% of theor., relative to example 69A) of the target compound.

LC-MS (method 4): Rₜ=1.37 min; MS (EIpos): m/z=468 [M+H]⁺.

### Example 93A

### Methyl-2-{[4-(8-fluoroquinoxalin-6-yl)-3-methyl-1-phenyl-1H-pyrazol-5-yl]amino}benzene carboxylate

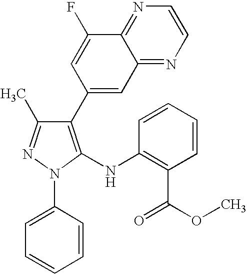

59 mg (0.065 mmol) of tris-(dibenzylidene-acetone)-dipalladium(0) and 44 mg (0.155 mmol) of tricyclohexylphosphine were added to 1.774 g of the raw product from example 91A. It was evacuated 5 times and was ventilated with argon in each case. Then 3.5 ml dioxane, 0.500 g (1.294 mmol) of the compound from example 72A and 1.7 ml of 1.27 M aqueous potassium phosphate solution were added. It was stirred for 2 h at 100° C. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 284 mg (48% of theor.) of the target compound.

LC-MS (method 2): Rₜ=2.36 min; MS (EIpos): m/z=454 [M+H]⁺.

### Example 94A

### 2,3-Difluoro-6-nitroaniline

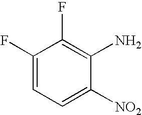

10.00 g (56.471 mmol) of 2,3,4-trifluornitrobenzene was dissolved in a 2M methanolic ammonia solution and heated in an autoclave for 2 h at 70° C. After cooling, the solvent was removed in a rotary evaporator and the residue was taken up in ethyl acetate. The organic phase was washed with water, dried over sodium sulfate and concentrated in a rotary evaporator. The resultant solid was recrystallized from 120 ml methanol/water (v/v=1:1). We obtained 7.21 g (73% of theor.) of the target compound.

GC-MS (method 6): Rₜ=4.10 min; MS (EIpos): m/z=174 [M]⁺.

¹H-NMR (400 MHz, DMSO-D₆): δ [ppm]=6.72 (dt, 1H), 7.53 (sbr, 2H), 7.93 (ddd, 1H).

### Example 95A

### 4-Bromo-2,3-difluoro-6-nitroaniline

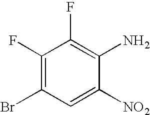

4.00 g (22.974 mmol) of the compound from example 94A was dissolved in 65 ml DMF and 5.15 g (28.948 mmol) of N-bromosuccinimide was added. The reaction solution was stirred for 1 h at 90° C. After cooling, the mixture was put in ice water and extracted with ethyl acetate. The combined organic phases were washed with water and saturated sodium chloride solution, dried over sodium sulfate and concentrated in a rotary evaporator. The residue was taken up in ethyl acetate and the organic phase was washed with water and saturated aqueous sodium chloride solution. The organic phase was dried over sodium sulfate and concentrated in a rotary evaporator. We obtained 5.63 g (97% of theor.) of the target compound.

GC-MS (method 6): Rₜ=5.30 min; MS (EIpos): m/z=252 [M]⁺.

¹H-NMR (400 MHz, DMSO-D₆): δ [ppm]=7.69 (sbr, 2H), 8.17 (dd, 1H).

### Example 96A

### 5-Bromo-3,4-difluorobenzene-1,2-diamine

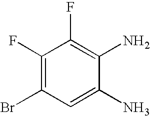

5.00 g (19.762 mmol) of the compound from example 95A was dissolved in 110 ml ethanol and 17.84 g (79.049 mmol) of tin(II) chloride dihydrate was added. The reaction solution was stirred overnight at 70° C. After cooling, it was diluted with water and it was made weakly alkaline with saturated aqueous sodium hydrogen carbonate solution. The ethanol was removed from the mixture in a rotary evaporator, and the mixture was filtered on Celite and washed again with ethyl acetate. The organic phase was removed and the aqueous phase was extracted three times with ethyl acetate. The combined organic phases were dried over sodium sulfate and concentrated in a rotary evaporator. The residue was dried under high vacuum. We obtained 3.96 g (87% of theor.) of the target compound.

LC-MS (method 10): Rₜ=0.81 min; MS (EIpos): m/z=223 [M+H]⁺.

¹H-NMR (400 MHz, DMSO-D₆): δ [ppm]=4.89 (sbr, 2H), 4.92 (sbr, 2H), 6.51 (dd, 1H).

### Example 97A

### 7-Bromo-5,6-difluoroquinoxaline

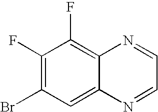

3.91 g (17.532 mmol) of the compound from example 96A was dissolved in 230 ml ethanol, 2.85 g (22.768 mmol) of 2,3-dihydroxy-1,4-dioxane was added and it was stirred overnight at room temperature. The reaction mixture was concentrated in a rotary evaporator, wherein the product crystallized out. It was cooled with ice water, the product was filtered off and dried under vacuum. We obtained 3.04 g (71% of theor.) of the target compound.

LC-MS (method 10): Rₜ=0.92 min; MS (EIpos): m/z=245 [M+H]⁺.

¹H-NMR (400 MHz, DMSO-D₆): δ [ppm]=8.46 (dd, 1H), 9.06-9.09 (m, 2H).

### Example 98A

### 5,6-Difluoro-7-(4,4,5,5-tetramethyl-1,3,2-dioxaborolan-2-yl)quinoxaline

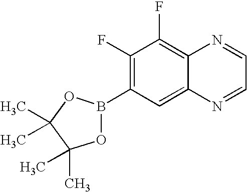

Under an argon atmosphere, 333 mg (0.364 mmol) of tris-(dibenzylidene-acetone)-dipalladium(0) and 245 mg (0.874 mmol) of tricyclohexylphosphine were dissolved in 40 ml dioxane. It was stirred for 30 min at room temperature. Then 1695 mg (6.675 mmol) of 4,4,4′4′5,5,5′5′-octamethyl-2,2′-bi-1,3,2-dioxaborolan, 1487 mg (6.069 mmol) of the compound from example 97A and 893 mg (9.103 mmol) potassium acetate were added and the mixture was stirred overnight at 80° C. After cooling, the reaction mixture was concentrated in a rotary evaporator, taken up in dichloromethane and filtered on Celite. The filtrate was concentrated in a rotary evaporator at reduced pressure and dried under high vacuum. We obtained 3.36 g of the raw product (purity 45% according to GC-MS), which was used in the next experiment without further purification.

GC-MS (method 6): Rₜ=6.80 min; MS (EIpos): m/z=292 [M]⁺.

### Example 99A

### Methyl-2-{[4-(7,8-difluoroquinoxalin-6-yl)-3-methyl-1-(2-methylphenyl)-1H-pyrazol-5-yl]amino}benzene carboxylate

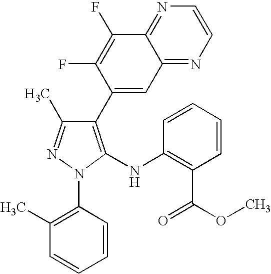

29 mg (0.031 mmol) of tris-(dibenzylidene-acetone)-dipalladium(0) and 21 mg (0.075 mmol) of tricyclohexylphosphine were added to 608 mg of the raw product from example 98A. It was evacuated 5 times and was ventilated with argon in each case. Then 2 ml dioxane, 250 mg (0.625 mmol) of the compound from example 69A and 840 μl a 1.27 M aqueous potassium phosphate solution were added. It was stirred overnight at 100° C. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 150 mg (19% of theor.) of the target compound with a purity according to LC-MS of 38%.

LC-MS (method 4): Rₜ=1.42 min; MS (EIpos): m/z=486 [M+H]⁺.

### Example 100A

### Methyl-2-{[4-(7,8-difluoroquinoxalin-6-yl)-3-methyl-1-phenyl-1H-pyrazol-5-yl]amino}benzene carboxylate

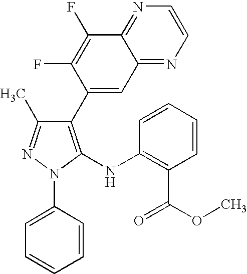

32 mg (0.035 mmol) of tris-(dibenzylidene-acetone)-dipalladium(0) and 23 mg (0.083 mmol) of tricyclohexylphosphine were added to 1.02 g of the raw product from example 98A. It was evacuated 5 times and ventilated with argon. Then 2 ml dioxane, 269 mg (0.696 mmol) of the compound from example 72A and 930 μl a 1.27 M potassium phosphate solution were added. It was stirred overnight at 100° C. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 148 mg (39% of theor.) of the target compound with a purity according to LC-MS of 86%.

LC-MS (method 2): Rₜ=2.46 min; MS (EIpos): m/z=472 [M+H]⁺.

### Example 101A

### N-(4-Bromopyridin-2-yl)-N′-hydroxyimidoformamide hydrochloride

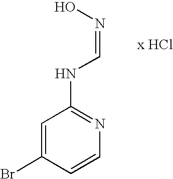

5.00 g (28.899 mmol) of 2-amino-4-bromopyridine was suspended in 10 ml isopropanol and 4.48 g (37.568 mmol) of dimethylformamide-dimethylacetal was added dropwise at room temperature. The mixture was boiled under reflux for 2 h (bath temperature 90° C.). It was cooled to 50° C., 2.61 g (37.568 mmol) of hydroxylamine hydrochloride was added and it was stirred for 10 min at 50° C. It was allowed to return to room temperature, the resultant precipitate was filtered off and the residue was dried under high vacuum. We obtained 4.37 g (57% of theor.) of the target compound.

LC-MS (method 10): Rₜ=0.67 min; MS (EIpos): m/z=216 [M+H]⁺.

¹H-NMR (400 MHz, DMSO-D₆): δ [ppm]=7.07 (dd, 1H), 7.32 (d, 1H), 7.81 (d, 1H), 8.03 (d, 1H), 8.45 (sbr, 1H), 9.55 (d, 1H), 10.24 (s, 1H).

### Example 102A

### 7-Bromo [1,2,4]triazolo[1,5-a]pyridine

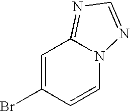

4.00 g (15.176 mmol) of the compound from example 101A was suspended in 60 ml THF. It was cooled on an ice bath and 3.51 g (16.694 mmol) of trifluoroacetic anhydride was dropwise added within 10 min, so that the internal temperature did not exceed 20° C. The ice bath was removed and the reaction mixture was stirred overnight at room temperature. 200 ml of saturated aqueous sodium hydrogen carbonate solution was added and it was extracted three times with tert.-butylmethyl ether. The combined organic phases were washed with saturated aqueous sodium hydrogen carbonate solution, dried over sodium sulfate and concentrated in a rotary evaporator. We obtained 2.85 g (76% of theor.) of the target compound at a purity of 80% according to LC-MS. For analytical purposes a small amount was purified by preparative HPLC (eluent: acetonitrile/water, gradient 10:90→90:10).

LC-MS (method 4) Rₜ=0.61 min; MS (EIpos): m/z=198 [M+H]⁺.

¹H-NMR (400 MHz, DMSO-D₆): δ [ppm]=7.39 (dd, 1H), 8.23-8.25 (m, 1H), 8.54 (s, 1H), 8.94 (d, 1H).

### Example 103A

### 7-(4,4,5,5-Tetramethyl-1,3,2-dioxaborolan-2-yl)[1,2,4]triazolo[1,5-a]pyridine

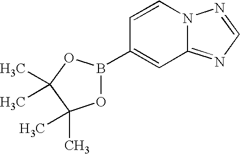

Under an argon atmosphere, 277 mg (0.303 mmol) of tris-(dibenzylidene-acetone)-dipalladium(0) and 204 mg (0.727 mmol) of tricyclohexylphosphine were dissolved in 30 ml dioxane. It was stirred for 30 min at room temperature. Then 1411 mg (5.555 mmol) of 4,4,4′4′5,5,5′5′-octamethyl-2,2′-bi-1,3,2-dioxaborolan, 1000 mg (5.050 mmol) of the compound from example 102A and 743 mg (7.575 mmol) potassium acetate were added and the mixture was stirred overnight at 80° C. After cooling, the reaction mixture was concentrated in a rotary evaporator, taken up in dichloromethane and filtered on Celite. The filtrate was concentrated in a rotary evaporator at reduced pressure and dried under high vacuum. We obtained 2415 mg of the raw product (purity 58% according to GC-MS), which was used in the next experiment without further purification.

GC-MS (method 6): Rₜ=6.33 min; MS (EIpos): m/z=245 [M]⁺.

### Example 104A

### Methyl-2-{[3-methyl-1-(2-methylphenyl)-4-([1,2,4]triazolo[1,5-a]pyridin-7-yl)-1H-pyrazol-5-yl]amino}benzene carboxylate

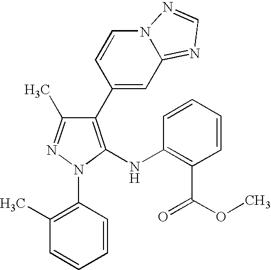

40 mg (0.044 mmol) of tris-(dibenzylidene-acetone)-dipalladium(0) and 29 mg (0.105 mmol) of tricyclohexylphosphine were added to 1225 mg of the raw product from example 103A. It was evacuated 5 times and ventilated with argon. Then 2.5 ml dioxane, 350 mg (0.874 mmol) of the compound from example 69A and 1.17 ml of 1.27 M aqueous potassium phosphate solution were added. It was stirred for 3 h at 100° C. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 205 mg (53% of theor.) of the target compound.

LC-MS (method 10): Rₜ=1.07 min; MS (EIpos): m/z=439 [M+H]⁺.

¹H-NMR (400 MHz, DMSO-D₆): δ [ppm]=2.16 (s, 3H), 2.47 (s, 3H), 3.80 (s, 3H), 6.46 (d, 1H), 6.63 (t, 1H), 7.16-7.32 (m, 5H), 7.40 (d, 1H), 7.68 (dd, 1H), 8.44 (s, 1H), 8.88 (d, 1H), 9.21 (s, 1H).

### Example 105A

Ethyl-[5-bromopyridin-2-yl)carbamothioyl]carbamate

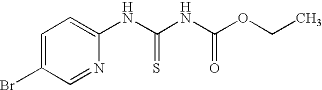

1.00 g (5.780 mmol) of 2-amino-5-bromopyridine was dissolved in 20 ml dioxane and 0.76 g (5.780 mmol) of ethylisothiocyanate-carbonate was added dropwise at room temperature. It was stirred at room temperature for 15 h and then the mixture was concentrated in a rotary evaporator. The residue was taken up in a little ethyl acetate and filtered on silica gel. It was washed again with ethyl acetate and the product-containing fractions were combined, dried over sodium sulfate and concentrated in a rotary evaporator. The residue was dried under high vacuum. We obtained 8.70 g (99% of theor.) of the target compound.

LC-MS (method 10): Rₜ=1.07 min; MS (EIpos): m/z=305 [M+H]⁺.

¹H-NMR (400 MHz, DMSO-D₆): δ [ppm]=1.26 (t, 3H), 4.23 (q, 2H), 8.13 (dd, 1H), 8.54 (d, 1H), 8.62 (sbr, 1H), 11.71 (sbr, 1H), 12.19 (s, 1H).

### Example 106A

### 6-Bromo[1,2,4]triazolo[1,5-a]pyridine-2-amine

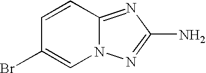

12.83 g (184.632 mmol) of hydroxylamine hydrochloride and 14.32 g (110.779 mmol) N,N-diisopropylamine were dissolved in 70 ml methanol/ethanol (v/v=1:1). 10.4 g of the compound from example 105A was added and the mixture was stirred at room temperature for 2 h and at 60° C. for 3 h. After cooling, water and ethyl acetate were added, the organic phase was removed and the aqueous phase was extracted twice with ethyl acetate. The combined organic phases were dried over sodium sulfate, concentrated in a rotary evaporator and the residue was dried under high vacuum. We obtained 7.02 g (96% of theor.) of the target compound.

GC-MS (method 6): Rₜ=6.11 min; MS (EIpos): m/z=212 [M]⁺.

¹H-NMR (400 MHz, DMSO-D₆): δ [ppm]=6.13 (s, 2H), 7.33 (d, 1H), 7.55 (dd, 1H), 8.92 (d, 1H).

### Example 107A

### 6-(4,4,5,5-Tetramethyl-1,3,2-dioxaborolan-2-yl) [1,2,4]triazolo[1,5-a]pyridine-2-amine

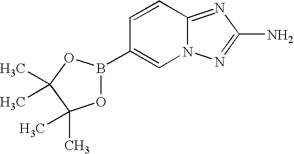

Under an argon atmosphere, 1.238 g (1.352 mmol) of tris-(dibenzylidene-acetone)-dipalladium(0) and 0.910 g (3.246 mmol) of tricyclohexylphosphine were dissolved in 140 ml dioxane. It was stirred for 30 min at room temperature. Then 6.296 g (24.792 mmol) of 4,4,4′4′5,5,5′5′-octamethyl-2,2′-bi-1,3,2-dioxaborolan, 4.860 g (22.539 mmol) of the compound from example 106A and 3.318 g (33.808 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 18.1 g of the raw product (purity 4% according to GC-MS), which was used in the next experiment without further purification.

GC-MS (method 6): Rₜ=7.49 min; MS (EIpos): m/z=260 [M]⁺.

### Example 108A

### Methyl-2-{[4-(2-amino [1,2,4]triazolo[1,5-a]pyridin-6-yl)-3-methyl-142-methylphenyl)-1H-pyrazol-5-yl]amino}benzene carboxylate

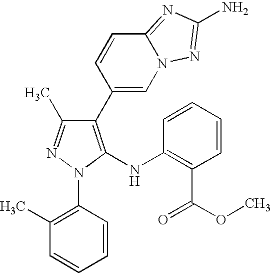

Under an argon atmosphere, 250 mg (0.625 mmol) of the compound from example 69A was dissolved in 5.5 ml DMF and 3000 mg of the raw product from example 107A and 1.3 ml (4.196 mmol) of 2N sodium carbonate solution were added. 43 mg (0.037 mmol) of tetrakis(triphenylphosphine)palladium(0) was added and the reaction mixture was heated at 110° C. for 4 h. After cooling, ethyl acetate was added and the organic phase was separated. The organic phase was washed with water, dried over sodium sulfate and concentrated in a rotary evaporator. The residue was purified by preparative HPLC (eluent: acetonitrile/water, gradient 10:90→90:10). We obtained 43 mg (15% of theor.) of the target compound.

LC-MS (method 4): Rₜ=1.12 min; MS (EIpos): m/z=454 [M+H]⁺.

### Example 109A

Ethyl-[(4-bromopyridin-2-yl)carbamothioyl]carbamate

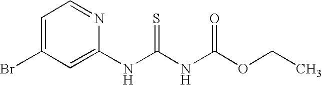

5.00 g (28.899 mmol) of 2-amino-4-bromopyridine was dissolved in 100 ml dioxane and 3.79 g (28.899 mmol) of ethylisothiocyanate-carbonate was added dropwise at room temperature. It was stirred at room temperature for 15 h and then the mixture was concentrated in a rotary evaporator. The residue was taken up in a little ethyl acetate and filtered on silica gel. It was washed again with ethyl acetate and the product-containing fractions were combined, dried over sodium sulfate and concentrated in a rotary evaporator. The residue was dried under high vacuum. We obtained 8.60 g (98% of theor.) of the target compound.

LC-MS (method 10): Rₜ=1.06 min; MS (EIpos): m/z=304 [M+H]⁺.

¹H-NMR (400 MHz, DMSO-D₆): δ [ppm]=1.27 (t, 3H), 4.23 (q, 2H), 7.53 (dd, 1H), 8.31 (d, 1H), 8.91 (sbr, 1H), 11.84 (sbr, 1H), 12.21 (s, 1H).

### Example 110A

### 7-Bromo[1,2,4]triazolo[1,5-a]pyridine-2-amine

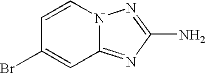

10.61 g (152.677 mmol) of hydroxylamine hydrochloride and 11.84 g (91.606 mmol) N,N-diisopropylamine were dissolved in 60 ml methanol/ethanol (v/v=1:1). 8.6 g (28.273 mmol) of the compound from example 109A was added and the mixture was stirred at room temperature for 2 h and at 60° C. for 3 h. After cooling, water and ethyl acetate were added, the organic phase was removed and the aqueous phase was extracted twice with ethyl acetate. The combined organic phases were dried over sodium sulfate, concentrated in a rotary evaporator and the residue was dried under high vacuum. We obtained 4.05 g (59% of theor.) of the target compound at a purity of 87% according to

LC-MS.

LC-MS (method 10): Rₜ=0.50 min; MS (EIpos): m/z=213 [M+H]⁺.

¹H-NMR (400 MHz, DMSO-D₆): δ [ppm]=6.13 (s, 2H), 7.02 (dd, 1H), 7.65 (dd, 1H), 8.49 (dd, 1H).

### Example 111A

### 7-(4,4,5,5-Tetramethyl-1,3,2-dioxaborolan-2-yl) [1,2,4]triazolo[1,5-a]pyridine-2-amine

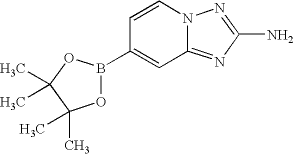

Under an argon atmosphere, 0.686 g (0.749 mmol) of tris-(dibenzylidene-acetone)-dipalladium(0) and 0.504 g (1.798 mmol) of tricyclohexylphosphine were dissolved in 75 ml dioxane. It was stirred for 30 min at room temperature. Then 3.488 g (13.734 mmol) of 4,4,4′4′5,5,5′5′-octamethyl-2,2′-bi-1,3,2-dioxaborolan, 2.660 g (12.486 mmol) of the compound from example 110A and 1.838 g (18.729 mmol) potassium acetate were added and the mixture was stirred overnight at 80° C. After cooling, dioxane was added to the reaction mixture and it was filtered on Celite. The filtrate was concentrated in a rotary evaporator at reduced pressure and dried under high vacuum. We obtained 9.300 g of the raw product (purity 25% according to GC-MS), which was used in the next experiment without further purification.

GC-MS (method 6): Rₜ=7.54 min; MS (EIpos): m/z=260 [M]⁺.

### Example 112A

### Methyl-2-{[442-amino [1,2,4]triazolo[1,5-a]pyridin-7-yl)-3-methyl-142-methylphenyl)-1H-pyrazol-5-yl]amino}benzene carboxylate

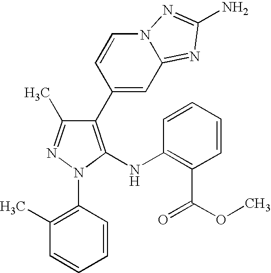

Under an argon atmosphere, 250 mg (0.625 mmol) of the compound from example 69A was dissolved in 5.5 ml DMF and 3000 mg of the raw product from example 111A and 1.3 ml (4.196 mmol) of 2N aqueous sodium carbonate solution were added. 43 mg (0.037 mmol) of tetrakis(triphenylphosphine)palladium(0) was added and the reaction mixture was heated for 4 h at 110° C. After cooling, ethyl acetate was added and the organic phase was separated. The organic phase was washed with water, dried over sodium sulfate and concentrated in a rotary evaporator. The residue was purified by preparative HPLC (eluent: acetonitrile/water, gradient 10:90→90:10). We obtained 159 mg (56% of theor.) of the target compound.

LC-MS (method 10): Rₜ=0.97 min; MS (EIpos): m/z=454 [M+H]⁺.

### Example 113A

### Methyl-2-{[4-(1-aminoisoquinolin-7-yl)-3-methyl-142-methylphenyl)-1H-pyrazol-5-yl]amino}benzene carboxylate

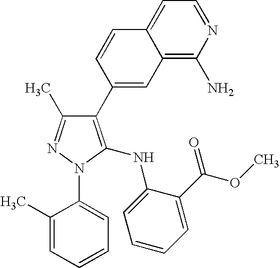

### Stage 1

Under an argon atmosphere, 500 mg (2.241 mmol) of 7-bromo-isoquinolin-1-amine [Rewinkel et al., Bioorg. Med. Chem. Lett. 1999, 9, 2837] was dissolved in 4 ml dioxane. Then 660 mg (6.72 mmol) potassium acetate, 146 mg (0.179 mmol) of 1,1′-bis-(diphenylphosphino)ferrocene-palladium(II) chloride-dichloromethane complex and 626 mg (2.465 mmol) of 4,4,4′4′5,5,5′5′-octamethyl-2,2′-bi-1,3,2-dioxaborolan were added. The reaction mixture was stirred overnight at an oil bath temperature of 130° C. After cooling, dichloromethane was added and it was filtered on kieselguhr. Then it was washed again with ethyl acetate. The filtrate was concentrated in a rotary evaporator at reduced pressure and dried under high vacuum. We obtained 776 mg of 7-(4,4,5,5-tetramethyl-1,3,2-dioxaborolan-2-yl)isoquinolin-1-amine [63%, (LC-MS)] as raw product. This was reacted subsequently without further purification.

### Stage 2

Under an argon atmosphere, 388 mg of the 7-(4,4,5,5-tetramethyl-1,3,2-dioxaborolan-2-yl)isoquinolin-1-amine raw product thus obtained, together with 383 mg (0.958 mmol) of example 69A, 32.2 mg (0.115 mmol) of tricyclohexylphosphine, 43.8 mg (0.048 mmol) of tris(dibenzylidene-acetone)-dipalladium and 1.63 ml (1.63 mmol) of 1M potassium phosphate solution, was taken up in 4 ml dioxane. Then it was reacted overnight at the reflux temperature. The volatile components were removed in a rotary evaporator and the residue was then purified by preparative HPLC (eluent: acetonitrile/water, gradient 10:90→90:10). We obtained 100 mg (23% of theor.) of the target compound.

LC-MS (method 4): Rₜ=1.06 min; MS (EIpos): m/z=464 [M+H]⁺.

### Example 114A

### 6-Bromo-8-fluoroquinoline

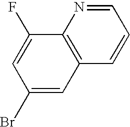

5.00 g (26.3 mmol) of 4-bromo-2-fluoroaniline, 5.31 g (57.89 mmol) glycerol and 9.89 g (43.94 mmol) of 3-nitrophenylsulfonic acid-sodium salt were prepared and homogenized. Then 25 ml of 70% sulfuric acid was added dropwise. The mixture was stirred overnight at 135° C. The cooled black reaction mixture was made alkaline, cautiously and with ice cooling, with 50% sodium hydroxide solution, and then filtered on a large bed of silica gel and kieselguhr. It was washed again with water and ethyl acetate. The phases collected were combined, then the organic phase was separated. The aqueous phase that remained was then extracted with ethyl acetate (2×). Then the organic phases thus obtained and the phase already separated previously were combined. It was dried over magnesium sulfate and the volatile components were removed in a rotary evaporator. The residue was finally purified by MPLC (Puriflash Analogix: 40M: isohexane/ethyl acetate=4/1). We obtained 3.56 g (60% of theor.) of the target compound.

¹H-NMR (400 MHz, DMSO-D₆): δ [ppm]=7.70 (dd, 2H), 7.89 (dd, 1H), 8.17 (s, 1H), 8.44 (d, 1H), 9.00 (d, 1H).

### Example 115A

### Methyl-2-{[4-(8-fluoroquinolin-6-yl)-3-methyl-1-phenyl-1H-pyrazol-5-yl]amino}benzene carboxylate

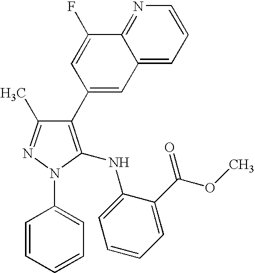

### Stage 1

Under an argon atmosphere, 1.00 g (4.42 mmol) of example 114A was dissolved in 20 ml dioxane. Then 1.302 g (13.27 mmol) potassium acetate, 289 mg (0.354 mmol) of 1,1′-bis-(diphenyl-phosphino)ferrocene-palladium(II) chloride-dichloromethane complex and 1.69 g (6.64 mmol) of 4,4,4′4′5,5,5′5′-octamethyl-2,2′-bi-1,3,2-dioxaborolan were added. The reaction mixture was stirred overnight at an oil bath temperature of 130° C. After cooling, dichloromethane was added and it was filtered on kieselguhr. Then it was washed again with ethyl acetate. The filtrate was concentrated in a rotary evaporator at reduced pressure and dried under high vacuum. We obtained 2.01 g of 8-fluoroquinolin-6-yl)boric acid [73%, (LC-MS)] as raw product. This was reacted subsequently without further purification.

### Stage 2

Under an argon atmosphere, 148 mg of the 8-fluoroquinolin-6-yl)boric acid raw product thus obtained, together with 200 mg (0.518 mmol) of example 72A, 174 mg (0.621 mmol) of tricyclohexylphosphine, 24 mg (0.026 mmol) of tris(dibenzylidene-acetone)-dipalladium and 0.880 ml (0.880 mmol) of 1M potassium phosphate solution, was taken up in 3 ml dioxane. Then it was reacted overnight at the reflux temperature. The volatile components were removed in a rotary evaporator and the residue was then purified by preparative HPLC (eluent: acetonitrile/water, gradient 10:90→90:10). We obtained 73 mg (31% of theor.) of the target compound.

LC-MS (method 10): Rₜ=1.21 min; MS (EIpos): m/z=453 [M+H]⁺.

### Example 116A

### 7-Bromo-4-methoxyquinoline

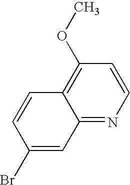

500 mg (2.06 mmol) of 7-bromo-4-chloroquinoline [De et al., J. Med. Chem. 1998, 41, 4918] was dissolved in 3 ml dioxane. Then 1.86 g (10.31 mmol) sodium methylate in 3 ml methanol was added and then reacted in a single mode microwave for 60 min at a temperature of 120° C. The mixture was filtered and washed with a little methanol. After drying, 250 mg (51% of theor.) of the target compound was obtained.

LC-MS (method 2): Rₜ=1.19 min; MS (EIpos): m/z=238 [M]⁺.

¹H-NMR (400 MHz, DMSO-D₆): δ [ppm]=7.09 (d, 1H), 7.70 (dd, 1H), 8.08 (d, 1H), 8.16 (d, 1H), 8.77 (s, 1H).

### Example 117A

### Methyl-2-{[4-(4-methoxyquinolin-7-yl)-3-methyl-1-(2-methylphenyl)-1H-pyrazol-5-yl]amino}benzene carboxylate

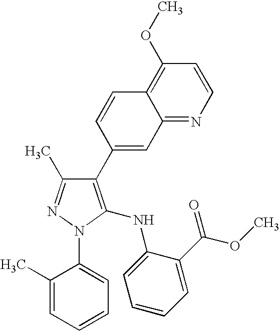

### Stage 1

Under an argon atmosphere, 250 mg (1.05 mmol) of example 116A was dissolved in 5 ml dioxane. Then 185 mg (1.89 mmol) potassium acetate, 68 mg (0.084 mmol) of 1,1′-bis-(diphenyl-phosphino)ferrocene-palladium(II) chloride-dichloromethane complex and 293 mg (1.16 mmol) of 4,4,4′4′5,5,5′5′-octamethyl-2,2′-bi-1,3,2-dioxaborolan were added. The reaction mixture was stirred overnight at an oil bath temperature of 130° C. After cooling, dichloromethane was added and it was filtered on kieselguhr. Then it was washed again with ethyl acetate. The filtrate was concentrated in a rotary evaporator at reduced pressure and dried under high vacuum. We obtained 560 mg (4-methoxyquinolin-7-yl)boric acid [94%, (LC-MS)] as raw product. This was reacted subsequently without further purification.

### Stage 2

Under an argon atmosphere, 202 mg of the (4-methoxyquinolin-7-yl)boric acid raw product thus obtained, together with 300 mg (0.749 mmol) of example 69A, 25.2 mg (0.090 mmol) of tricyclohexylphosphine, 34.3 mg (0.037 mmol) of tris(dibenzylidene-acetone)-dipalladium and 1.274 ml (1.274 mmol) of 1M aqueous potassium phosphate solution, was taken up in 7.5 ml dioxane. Then it was reacted overnight at the reflux temperature. The volatile components were removed in a rotary evaporator and the residue was then purified by preparative HPLC (eluent: acetonitrile/water, gradient 10:90→90:10). We obtained 130 mg (36% of theor.) of the target compound.

LC-MS (method 10): Rₜ=0.96 min; MS (EIpos): m/z=479 [M+H]⁺.

¹H-NMR (400 MHz, DMSO-D₆): δ [ppm]=2.17 (s, 3H), 2.45 (s, 3H), 3.79 (s, 3H), 4.01 (s, 3H), 6.47 (d, 1H), 6.59 (t, 1H), 6.97 (d, 1H), 7.15 (t, 1H), 7.23 (mc, 1H), 7.27-7.35 (m, 2H), 7.42 (d, 1H), 7.66 (d, 2H), 7.96 (s, 1H), 8.04 (d, 1H), 8.68 (d, 1H), 9.19 (s, 1H).

### Example 118A

### 7-Bromo-5-fluoroquinazoline

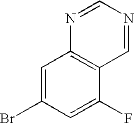

1000 mg (4.53 mmol) of 4-bromo-2,6-difluorobenzaldehyde [Wang et al., Org. Lett. 2007, 9, 5629] was taken up in 30 ml acetonitrile. Then it was rinsed with argon, 659 mg (6.34 mmol) potassium carbonate and then 800 mg activated molecular sieve (4 Å) were added. Then it was stirred overnight at the reflux temperature. After it had cooled, the reaction mixture was filtered on Celite. It was washed again with ethyl acetate. Then the volatile components were removed in a rotary evaporator. The residue was finally purified by MPLC (Puriflash Analogix: 40M: isohexane/ethyl acetate=4/1). We obtained 331 mg (32% of theor.) of the target compound.

GC-MS: (method 6): Rₜ=4.57 min; MS (EIpos): m/z=228 [M+H]⁺.

¹H-NMR (400 MHz, DMSO-D₆): δ [ppm]=7.96 (dd, 1H), 8.18 (s, 1H), 9.42 (s, 1H), 9.76 (s, 1H).

### Example 119A

### Methyl-2-{[4-(5-fluoroquinazolin-7-yl)-3-methyl-1-(2-methylphenyl)-1H-pyrazol-5-yl]amino}benzoate

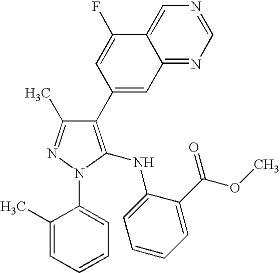

Under an argon atmosphere, 331 mg (1.46 mmol) of example 118A was dissolved in 10 ml dioxane. Then 429 mg (4.37 mmol) potassium acetate, 95.2 mg (0.117 mmol) of 1,1′-bis-(diphenylphosphino)-ferrocene palladium(II) chloride-dichloromethane complex and 407 mg (1.64 mmol) of 4,4,4′4′5,5,5′5′-octamethyl-2,2′-bi-1,3,2-dioxaborolan were added. The reaction mixture was stirred overnight at an oil bath temperature of 110° C. After cooling, dichloromethane was added and it was filtered on kieselguhr. It was washed again with ethyl acetate. Then the filtrate was concentrated in a rotary evaporator at reduced pressure and dried under high vacuum. Under an argon atmosphere, the raw product thus obtained, together with 862 mg (2.16 mmol) of example 69A, 72.5 mg (0.259 mmol) of tricyclohexylphosphine, 98.6 mg (0.108 mmol) of tris(dibenzylidene-acetone)-dipalladium and 3.67 ml (3.67 mmol) of 1M potassium phosphate solution, was taken up in 10 ml dioxane. Then it was reacted overnight at 100° C. The reaction mixture was concentrated by evaporation and was then submitted to column filtration on a silica gel bed. The volatile components were removed in a rotary evaporator and the reaction mixture was separated by preparative HPLC (eluent: acetonitrile/water, gradient 10:90→90:10). In order to separate a by-product, it was finally purified again by MPLC (Puriflash Analogix: 40M: isohexane/ethyl acetate=10/1->1/1). We obtained 155 mg (15% of theor., relative to example 69A) of the target compound.

LC-MS (method 10): Rₜ=1.24 min; MS (EIpos): m/z=468 [M+H]⁺.

¹H-NMR (400 MHz, DMSO-D₆): δ [ppm]=2.18 (s, 3H), 3.81 (s, 3H), 6.46 (d, 1H), 6.61 (t, 1H), 7.16 (mc, 1H), 7.25 (m, 1H), 7.29-7.35 (m, 2H), 7.42 (d, 1H), 7.67 (dd, 1H), 7.74 (d, 1H), 7.91 (s, 1H), 9.25 (s, 1H), 9.32 (s, 1H), 9.63 (s, 1H).

### Example 120A

### 3-Methyl-1-[2-(trifluoromethyl)phenyl]-1H-pyrazol-5-amine

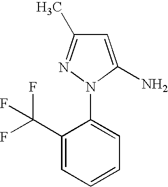

2.789 g (15.83 mmol) of 2-(trifluoromethyl)-phenylhydrazine was prepared in 15 ml of 1N hydrochloric acid and 1.378 g (16.781 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. The product was dried at high vacuum and purified by preparative HPLC (eluent: acetonitrile/water, gradient 20:80→90:10). We obtained 2.500 g (60% of theor., purity 91% according to HPLC) of the target compound.

LC-MS (method 10): Rₜ=1.09 min; MS (EIpos): m/z=242 [M+H]⁺.

### Example 121A

### Methyl-2-({3-methyl-1-[2-(trifluoromethyl)phenyl]-1H-pyrazol-5-yl}amino)benzoate

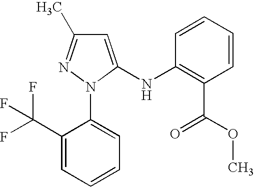

Under an argon atmosphere, 2.430 g (10.074 mmol) of 3-methyl-1-[2-(trifluoromethyl)phenyl]-1H-pyrazol-5-amine (compound from example 120A) was dissolved in 30 ml toluene and 75 mg (0.336 mmol) of palladium(II) acetate and 0.362 g (0.672 mmol) bis-[2-diphenylphosphino)-phenyl]ether were added. The mixture was stirred for 5 min at room temperature. Then 1.444 g (6.716 mmol) of methyl-2-bromobenzoate and 3.064 g (9.402 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by preparative HPLC (eluent: acetonitrile/water, gradient 20:80→90:10). We obtained 2.500 g (65% of theor.) of the target compound.

LC-MS (method 10): Rₜ=1.23 min; MS (EIpos): m/z=376 [M+H]⁺.

¹H-NMR (400 MHz, DMSO-D₆): δ [ppm]=2.23 (s, 3H), 3.72 (s, 3H), 6.22 (s, 1H), 6.82-6.86 (m 1H), 7.20 (d, 1H), 7.46-7.50 (m, 1H), 7.60 (d, 1H), 7.75 (t, 1H), 7.80-7.85 (m, 2H), 7.93-7.96 (m, 1H), 9.27 (s, 1H).

### Example 122A

### Methyl-2-({4-bromo-3-methyl-1-[2-(trifluoromethyl)phenyl]-1H-pyrazol-5-yl}amino)benzoate

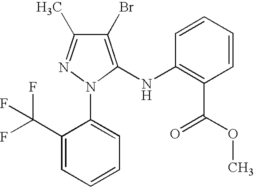

2.375 g (6.327 mmol) of the compound from example 121A was dissolved in 35 ml dichloromethane and, while cooling with ice, 0.905 g (3.164 mmol) of 1,3-dibromo-5,5-dimethylhydantoin was added in portions. Reaction testing by HPLC showed that reaction was completed after 60 min. Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 2.800 g (97% of theor.) of the target compound.

LC-MS (method 2): Rₜ=2.72 min; MS (EIpos): m/z=456 [M+H]⁺.

¹H-NMR (400 MHz, DMSO-D₆): δ [ppm]=2.26 (s, 3H), 3.79 (s, 3H), 6.58 (d, 1H), 6.81 (dt, 1H), 7.41 (dt, 1H), 7.68-7.73 (m, 2H), 7.76-7.80 (m, 2H), 7.90 (d, 1H), 9.04 (s, 1H).

### Example 123A

### Methyl-2-({4-(8-fluoroquinoxalin-6-yl)-3-methyl-1-[2-(trifluoromethyl)phenyl]-1H-pyrazol-5-yl}amino)benzoate

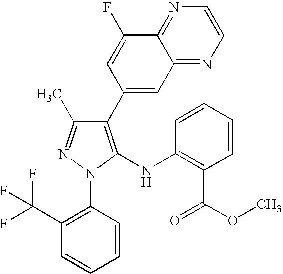

53 mg (0.058 mmol) of tris-(dibenzylidene-acetone)-dipalladium(0) and 39 mg (0.138 mmol) of tricyclohexylphosphine were added to 1.186 g of the raw product from example 91A. It was evacuated 5 times and ventilated with argon. Then 3.0 ml dioxane, 0.524 g (1.154 mmol) of the compound from example 122A and 1.5 ml of 1.27 M aqueous potassium phosphate solution were added. It was stirred for 2 h at 100° C. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 400 mg (53% of theor., purity 80%) of the target compound.

LC-MS (method 10): Rₜ=1.22 min; MS (EIpos): m/z=522 [M+H]⁺.

### Example 124A

### 1-(2,5-Dimethylphenyl)-3-methyl-1H-pyrazol-5-amine

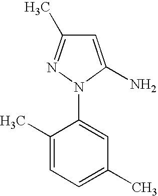

4.00 g (23.167 mmol) of 2,5-dimethylphenylhydrazine hydrochloride was suspended in 23 ml of 1N hydrochloric acid and 2.016 g (24.557 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. We obtained 4.301 g (92% of theor.) of the target compound.

LC-MS (method 2): Rₜ=1.05 min; MS (EIpos): m/z=202 [M+H]⁺.

¹H-NMR (400 MHz, DMSO-D₆): δ [ppm]=2.00 (s, 3H) 2.03 (s, 3H), 2.29 (s, 3H), 4.82 (s, 2H), 5.20 (s, 1H), 7.01 (s, 1H), 7.13 (dd, 1H), 7.21 (d, 1H).

### Example 125A

### Methyl-2-{[1(2,5-dimethylphenyl)-3-methyl-1H-pyrazol-5-yl]amino}benzoate

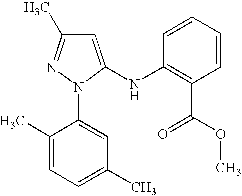

Under an argon atmosphere, 4.200 g (20.867 mmol) of the compound from example 124A was dissolved in 60 ml toluene and 156 mg (0.696 mmol) of palladium(II) acetate and 0.749 g (1.391 mmol) bis[2-diphenylphosphino)-phenyl]-ether were added. The mixture was stirred for 5 min at room temperature. Then 2.992 g (13.911 mmol) of methyl-2-bromobenzoate and 6.346 g (19.476 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by preparative HPLC (eluent: acetonitrile/water, gradient 10:90→90:10). We obtained 4.65 g (66% of theor.) of the target compound.

LC-MS (method 10): Rₜ=1.29 min; MS (EIpos): m/z=336 [M+H]⁺.

¹H-NMR (400 MHz, DMSO-D₆): δ [ppm]=1.98 (s, 3H), 2.23 (s, 3H), 2.29 (s, 3H), 3.73 (s, 3H), 6.19 (s, 1H), 6.82-6.86 (m 1H), 7.12 (s, 1H), 7.19 (dd, 1H), 7.24-7.27 (m, 2H), 7.47-7.51 (m, 1H), 7.83 (dd, 1H), 9.28 (s, 1H).

### Example 126A

### Methyl-2-{[4-bromo-1-(2,5-dimethylphenyl)-3-methyl-1H-pyrazol-5-yl]amino}benzoate

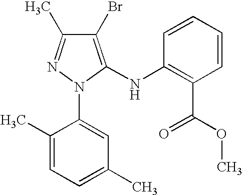

4.568 g (13.620 mmol) of the compound from example 125A was dissolved in 75 ml dichloromethane and, while cooling with ice, 1.947 g (6.810 mmol) of 1,3-dibromo-5,5-dimethylhydantoin was added in portions. Reaction testing by HPLC showed that reaction was completed after 30 min. Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 5.402 g (96% of theor.) of the target compound.

LC-MS (method 10): Rₜ=1.37 min; MS (EIpos): m/z=414 [M+H]⁺.

¹H-NMR (400 MHz, DMSO-D₆): δ [ppm]=2.02 (s, 3H), 2.22 (s, 3H), 2.26 (s, 3H), 3.80 (s, 3H), 6.54 (d, 1H), 6.79 (t, 1H), 7.11-7.19 (m, 3H), 7.38-7.42 (m, 1H), 7.79 (dd, 1H), 8.97 (s, 1H).

### Example 127A

### Methyl-2-{[1-(2,5-dimethylphenyl)-4-(8-fluoroquinoxalin-6-yl)-3-methyl-1H-pyrazol-5-yl]amino}benzoate

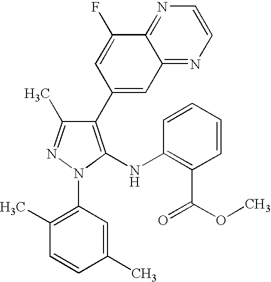

55 mg (0.060 mmol) of tris-(dibenzylidene-acetone)-dipalladium(0) and 41 mg (0.145 mmol) of tricyclohexylphosphine were added to 1.103 g of the raw product from example 91A. It was evacuated 5 times and ventilated with argon. Then 3.0 ml dioxane, 0.500 g (1.207 mmol) of the compound from example 126A and 1.6 ml of 1.27 M potassium phosphate solution were added. It was stirred overnight at 100° C. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 395 mg (68% of theor.) of the target compound.

LC-MS (method 10): Rₜ=1.30 min; MS (EIpos): m/z=482 [M+H]⁺.

### Example 128A

### 1-(2,4-Difluorophenyl)-3-methyl-1H-pyrazol-5-amine

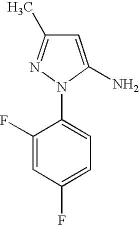

5.040 g (27.911 mmol) 2,4-difluorophenylhydrazine hydrochloride was suspended in 28 ml of 1N hydrochloric acid and 2.429 g (29.586 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. The residue was purified by silica-gel chromatography (eluent: iso-hexane/ethyl acetate, gradient 85:15→10:90). We obtained 4.740 g (81% of theor.) of the target compound.

LC-MS (method 4): Rₜ=0.52 min; MS (EIpos): m/z=210 [M+H]⁺.

¹H-NMR (400 MHz, DMSO-D₆): δ [ppm]=2.03 (s, 3H), 5.14 (sbr, 2H), 5.22 (s, 1H), 7.15-7.21 (m, 1H), 7.14-7.50 (m, 2H).

### Example 129A

### Methyl-2-{[1-(2,4-difluorophenyl)-3-methyl-1H-pyrazol-5-yl]amino}benzoate

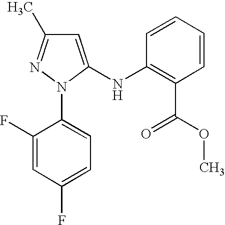

Under an argon atmosphere, 2.000 g (20.867 mmol) of the compound from example 128A was dissolved in 30 ml toluene and 177 mg (0.319 mmol) of palladium(II) acetate and 0.849 g (0.637 mmol) bis[2-diphenylphosphino)-phenyl]-ether were added. The mixture was stirred for 5 min at room temperature. Then 1.371 g (6.373 mmol) of methyl-2-bromobenzoate and 7.19 g (8.923 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by preparative HPLC (eluent: acetonitrile/water, gradient 10:90→90:10). We obtained 2.23 g (68% of theor.) of the target compound.

LC-MS (method 4): Rₜ=1.36 min; MS (EIpos): m/z=344 [M+H]⁺.

¹H-NMR (400 MHz, DMSO-D₆): δ [ppm]=2.24 (s, 3H), 3.79 (s, 3H), 6.26 (s, 1H), 6.84 (t, 1H) 7.08 (d, 1H), 7.22-7.27 (m, 1H), 7.43-7.48 (m, 1H), 7.65 (dt, 1H), 7.84 (dd, 1H), 9.37 (s, 1H).

### Example 130A

### Methyl-2-{[4-bromo-1-(2,4-difluorophenyl)-3-methyl-1H-pyrazol-5-yl]amino}benzoate

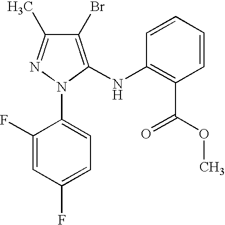

2.221 g (6.470 mmol) of the compound from example 129A was dissolved in 35 ml dichloromethane and, while cooling with ice, 0.925 g (3.235 mmol) of 1,3-dibromo-5,5-dimethylhydantoin was added in portions. Reaction testing by HPLC showed that reaction was completed after 30 min. Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 2.540 g (92% of theor.) of the target compound.

LC-MS (method 10): Rₜ=1.29 min; MS (EIpos): m/z=422 [M+H]⁺.

¹H-NMR (400 MHz, DMSO-D₆): δ [ppm]=2.27 (s, 3H), 3.83 (s, 3H), 6.49 (d, 1H), 6.81 (t, 1H) 7.19-7.24 (m, 1H), 7.36-7.40 (m, 1H), 7.46-7.51 (m, 1H), 7.68 (dt, 1H), 7.82 (dd, 1H), 9.09 (s, 1H).

### Example 131A

### Methyl-2-{[1-(2,4-difluorophenyl)-4-(8-fluoroquinoxalin-6-yl)-3-methyl-1H-pyrazol-5-yl]amino}benzoate

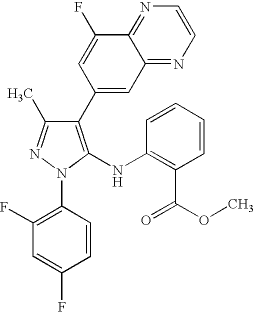

54 mg (0.059 mmol) of tris-(dibenzylidene-acetone)-dipalladium(0) and 40 mg (0.142 mmol) of tricyclohexylphosphine were added to 1.082 g of the raw product from example 91A. It was evacuated 5 times and ventilated with argon. Then 3.0 ml dioxane, 0.500 g (1.184 mmol) of the compound from example 130A and 1.6 ml of 1.27 M aqueous potassium phosphate solution were added. It was stirred at 100° C. for 5 h. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 488 mg (84% of theor.) of the target compound.

LC-MS (method 10): Rₜ=1.30 min; MS (EIpos): m/z=490 [M+H]⁺.

### Example 132A

### 1-(3-Fluoro-2-methylphenyl)-3-methyl-1H-pyrazol-5-amine

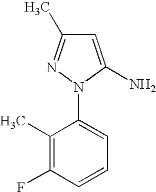

2.000 g (11.324 mmol) of 2-methyl-3-fluorophenylhydrazine hydrochloride was suspended in 11 ml of 1N hydrochloric acid and 0.986 g (12.003 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. We obtained 1.92 g (75% of theor., purity 91% according to HPLC) of the target compound.

LC-MS (method 10): Rₜ=0.53 min; MS (EIpos): m/z=206 [M+H]⁺.

¹H-NMR (400 MHz, DMSO-D₆): δ [ppm]=1.97 (d, 3H), 2.04 (s, 3H), 5.00 (sbr, 2H), 5.22 (s, 1H), 7.09 (d, 1H), 7.22-7.35 (m, 2H).

### Example 133A

### Methyl-2-{[1-(3-fluoro-2-methylphenyl)-3-methyl-1H-pyrazol-5-yl]amino}benzoate

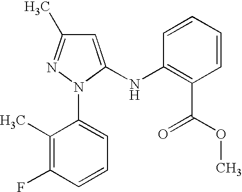

Under an argon atmosphere, 1.90 g (9.258 mmol) of the compound from example 132A was dissolved in 26 ml toluene and 69 mg (0.309 mmol) of palladium(II) acetate and 0.332 g (0.617 mmol) bis-[2-diphenylphosphino)-phenyl]ether were added. The mixture was stirred for 5 min at room temperature. Then 1.327 g (6.172 mmol) of methyl-2-bromobenzoate and 2.815 g (8.640 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by silica-gel chromatography (eluent: iso-hexane/ethyl acetate, gradient 90:10→10:90). We obtained 1.80 g (57% of theor.) of the target compound.

LC-MS (method 10): Rₜ=1.26 min; MS (EIpos): m/z=340 [M+H]⁺.

¹H-NMR (400 MHz, DMSO-D₆): δ [ppm]=1.95 (d, 3H), 2.24 (s, 3H), 3.74 (s, 3H), 6.24 (s, 1H), 6.85 (dt, 1H) 7.20-7.22 (m, 2H), 7.29-7.39 (m, 2H), 7.46-7.51 (m, 1H), 7.84 (dd, 1H), 9.30 (s, 1H).

### Example 134A

### Methyl-2-{[4-bromo-1-(3-fluoro-2-methylphenyl)-3-methyl-1H-pyrazol-5-yl]amino}benzoate

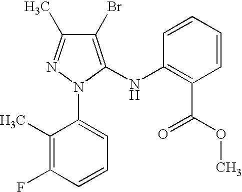

1.686 g (4.969 mmol) of the compound from example 133A was dissolved in 30 ml dichloromethane and, while cooling with ice, 0.710 g (2.484 mmol) of 1,3-dibromo-5,5-dimethylhydantoin was added in portions. Reaction testing by HPLC showed that reaction was completed after 20 min Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 5.402 g (92% of theor.) of the target compound.

LC-MS (method 2): Rₜ=2.78 min; MS (EIpos): m/z=418 [M+H]⁺.

¹H-NMR (400 MHz, DMSO-D₆): δ [ppm]=2.01 (d, 3H), 2.27 (s, 3H), 3.81 (s, 3H), 6.52 (d, 1H), 6.77-6.81 (m, 1H), 7.22-7.31 (m, 3H), 7.37-7.41 (m, 1H), 7.79 (dd, 1H), 9.00 (s, 1H).

### Example 135A

### Methyl-2-{[4-(8-fluoroquinoxalin-6-yl)-1-(3-fluoro-2-methylphenyl)-3-methyl-1H-pyrazol-5-yl]amino}benzoate

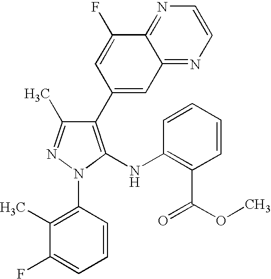

36 mg (0.039 mmol) of tris-(dibenzylidene-acetone)-dipalladium(0) and 26 mg (0.094 mmol) of tricyclohexylphosphine were added to 537 mg of the raw product from example 91A. It was evacuated 5 times and ventilated with argon. Then 2 ml dioxane, 328 mg (0.784 mmol) of the compound from example 134A and 1.05 ml of 1.27 M aqueous potassium phosphate solution were added. It was stirred at 100° C. for 5 h. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 277 mg (68% of theor.) of the target compound.

LC-MS (method 10): Rₜ=1.26 min; MS (EIpos): m/z=486 [M+H]⁺.

### Example 136A

### 1-(2,5-Difluorophenyl)-3-methyl-1H-pyrazol-5-amine

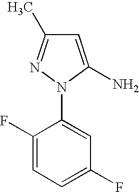

5.00 g (34.692 mmol) of 2,5-difluorophenylhydrazine was suspended in 35 ml of 1N hydrochloric acid and 3.019 g (36.774 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. We obtained 1.92 g (88% of theor.) of the target compound.

LC-MS (method 4): Rₜ=0.60 min; MS (EIpos): m/z=210 [M+H]⁺.

### Example 137A

### Methyl-2-{[1-(2,5-difluorophenyl)-3-methyl-1H-pyrazol-5-yl]amino}benzoate

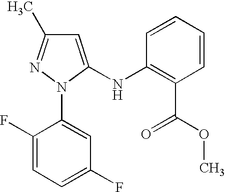

Under an argon atmosphere, 6.830 g (30.363 mmol) of the compound from example 136A was dissolved in 90 ml toluene and 227 mg (1.012 mmol) of palladium(II) acetate and 1.090 g (2.024 mmol) bis[2-diphenylphosphino)-phenyl]-ether were added. The mixture was stirred for 5 min at room temperature. Then 4.353 g (20.242 mmol) of methyl-2-bromobenzoate and 9.234 g (28.339 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by silica-gel chromatography (eluent: iso-hexane/ethyl acetate, gradient 90:10→10:90). We obtained 8.100 g (78% of theor.) of the target compound.

LC-MS (method 10): Rₜ=1.19 min; MS (EIpos): m/z=344 [M+H]⁺.

¹H-NMR (400 MHz, DMSO-D₆): δ [ppm]=2.25 (s, 3H), 3.80 (s, 3H), 6.28 (s, 1H), 6.84 (t, 1H) 7.07 (d, 1H), 7.36-7.57 (m, 4H), 7.85 (dd, 1H), 9.42 (s, 1H).

### Example 138A

### Methyl-2-{[4-bromo-1-(2,5-difluorophenyl)-3-methyl-1H-pyrazol-5-yl]amino}benzoate

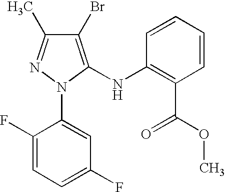

4.00 g (11.65 mmol) of the compound from example 137A was dissolved in 65 ml dichloromethane and, while cooling with ice, 1.67 g (5.825 mmol) of 1,3-dibromo-5,5-dimethylhydantoin was added in portions. Reaction testing by HPLC showed that reaction was completed after 20 min. Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 4.50 g (89% of theor.) of the target compound.

LC-MS (method 10): Rₜ=1.33 min; MS (EIpos): m/z=421 [M+H]⁺.

¹H-NMR (400 MHz, DMSO-D₆): δ [ppm]=2.28 (s, 3H), 3.84 (s, 3H), 6.48 (d, 1H), 6.81 (t, 1H), 7.33-7.49 (m, 2H), 7.57-7.62 (m, 1H), 7.82 (dd, 1H), 9.13 (s, 1H).

### Example 139A

### Methyl-2-{[1-(2,5-difluorophenyl)-4-(8-fluoroquinoxalin-6-yl)-3-methyl-1H-pyrazol-5-yl]amino}benzoate

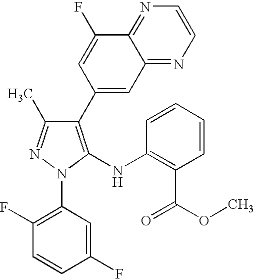

54 mg (0.059 mmol) of tris-(dibenzylidene-acetone)-dipalladium(0) and 40 mg (0.142 mmol) of tricyclohexylphosphine were added to 1.082 mg of the raw product from example 91A. It was evacuated 5 times and was ventilated with argon in each case. Then 3 ml dioxane, 500 mg (1.184 mmol) of the compound from example 138A and 1.59 ml of 1.27 M aqueous potassium phosphate solution were added. It was stirred for 2 h at 100° C. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 430 mg (74% of theor.) of the target compound.

LC-MS (method 10): Rₜ=1.20 min; MS (EIpos): m/z=490 [M+H]⁺.

### Example 140A

### 1-(4-Fluoro-2-methylphenyl)-3-methyl-1H-pyrazol-5-amine

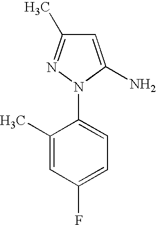

15.413 g (87.267 mmol) of 4-fluoro-2-methylphenylhydrazine hydrochloride (for preparation see WO 2007/077961 p. 294 example 107) was suspended in 90 ml of 1N hydrochloric acid and 7.595 g (92.503 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. The residue was purified by silica-gel chromatography (eluent: iso-hexane/ethyl acetate, gradient 70:30→30:70). We obtained 10.400 g (58% of theor.) of the target compound.

LC-MS (method 4): Rₜ=0.47 min; MS (EIpos): m/z=206 [M+H]⁺.

¹H-NMR (400 MHz, DMSO-D₆): δ [ppm]=2.03 (s, 3H), 2.04 (s, 3H), 4.91 (s, 2H), 5.21 (s, 1H), 7.07-7.12 (m, 1H) 7.19-7.25 (m, 2H).

### Example 141A

### Methyl-2-{[1-(4-fluoro-2-methylphenyl)-3-methyl-1H-pyrazol-5-yl]amino}benzoate

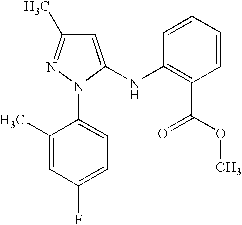

Under an argon atmosphere, 5.00 g (24.36 mmol) of the compound from example 140A was dissolved in 70 ml toluene and 182 mg (0.812 mmol) of palladium(II) acetate and 0.875 g (1.624 mmol) bis-[2-diphenylphosphino)-phenyl]ether were added. The mixture was stirred for 5 min at room temperature. Then 3.493 g (16.241 mmol) of methyl-2-bromobenzoate and 7.409 g (22.738 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by preparative HPLC (eluent: acetonitrile/water, gradient 10:90→90:10). We obtained 8.100 g (78% of theor.) of the target compound.

LC-MS (method 10): Rₜ=1.24 min; MS (EIpos): m/z=340 [M+H]⁺.

¹H-NMR (400 MHz, DMSO-D₆): δ [ppm]=2.02 (s, 3H), 2.23 (s, 3H), 3.74 (s, 3H), 6.21 (s, 1H), 6.84 (t, 1H) 7.16 (dt, 1H), 7.23 (d, 1H), 7.27 (dd, 1H), 7.38 (dd, 1H), 7.49 (dt, 1H), 7.84 (dd, 1H), 9.28 (s, 1H).

### Example 142A

### Methyl-2-{[4-bromo-1-(4-fluoro-2-methylphenyl)-3-methyl-1H-pyrazol-5-yl]amino}benzoate

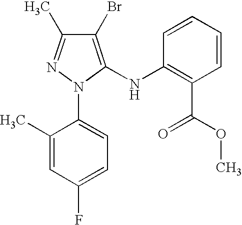

5.006 g (14.750 mmol) of the compound from example 141A was dissolved in 80 ml dichloromethane and, while cooling with ice, 2.109 g (7.375 mmol) of 1,3-dibromo-5,5-dimethylhydantoin was added in portions. Reaction testing by HPLC showed that reaction was completed after 20 min Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 5.821 g (94% of theor.) of the target compound.

LC-MS (method 4): Rₜ=1.52 min; MS (EIpos): m/z=418 [M+H]⁺.

¹H-NMR (400 MHz, DMSO-D₆): δ [ppm]=2.08 (s, 3H), 2.26 (s, 3H), 3.81 (s, 3H), 6.54 (d, 1H), 6.79 (dt, 1H) 7.09 (dt, 1H), 7.20 (dd, 1H), 7.37-7.44 (m, 2H), 7.79 (dd, 1H), 8.97 (s, 1H).

### Example 143A

### Methyl-2-{[4-(8-fluoroquinoxalin-6-yl)-1-(4-fluoro-2-methylphenyl)-3-methyl-1H-pyrazol-5-yl]amino}benzoate

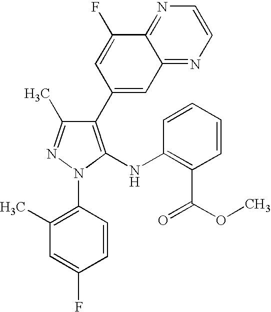

49 mg (0.054 mmol) of tris-(dibenzylidene-acetone)-dipalladium(0) and 36 mg (0.129 mmol) of tricyclohexylphosphine were added to 1.474 mg of the raw product from example 91A. It was evacuated 5 times and ventilated with argon. Then 3 ml dioxane, 450 mg (1.076 mmol) of the compound from example 142A and 1.44 ml of 1.27 M aqueous potassium phosphate solution were added. It was stirred at 100° C. for 5 h. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 430 mg (82% of theor.) of the target compound.

LC-MS (method 1): Rₜ=2.67 min; MS (EIpos): m/z=486 [M+H]⁺.

### Example 144A

### 1-(2-Fluoro-6-methylphenyl)-3-methyl-1H-pyrazol-5-amine

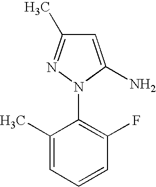

3.682 g (18.428 mmol) of 6-fluoro-2-methylphenylhydrazine hydrochloride (preparation similar to WO 2007/077961 p. 294 example 107) was suspended in 20 ml of 1N hydrochloric acid and 1.603 g (19.534 mmol) of 3-aminocrotonic nitrile was added. The mixture was stirred for 18 h at 100° C. After cooling, the pH value of the mixture was adjusted with 1N sodium hydroxide solution to pH>12. It was extracted with dichloromethane three times. The combined organic phases were washed with saturated aqueous sodium chloride solution, dried over sodium sulfate and concentrated in a rotary evaporator at reduced pressure. We obtained 3.88 g (100% of theor.) of the target compound.

LC-MS (method 10): Rₜ=0.51 min; MS (EIpos): m/z=206 [M+H]⁺.

¹H-NMR (400 MHz, DMSO-D₆): δ [ppm]=2.02 (s, 3H), 2.04 (s, 3H), 4.98 (s, 2H), 5.22 (s, 1H), 7.15-7.19 (m, 2H) 7.36-7.41 (m, 1H).

### Example 145A

### Methyl-2-{[1-(2-fluoro-6-methylphenyl)-3-methyl-1H-pyrazol-5-yl]amino}benzoate

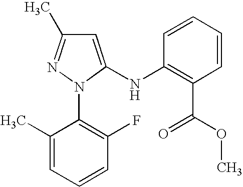

Under an argon atmosphere, 2.00 g (9.745 mmol) of the compound from example 144A was dissolved in 30 ml toluene and 73 mg (0.325 mmol) of palladium(II) acetate and 0.350 g (0.650 mmol) bis[2-diphenylphosphino)-phenyl]-ether were added. The mixture was stirred for 5 min at room temperature. Then 1.397 g (6.497 mmol) of methyl-2-bromobenzoate and 2.963 g (9.095 mmol) cesium carbonate were added and the mixture was stirred overnight at 95° C. After cooling, it was filtered on silica gel, the suction filter cake was washed with ethyl acetate and the filtrate was concentrated by evaporation. The residue was purified by silica-gel chromatography (eluent: iso-hexane/ethyl acetate, gradient 90:10→05:95). We obtained 2.26 g (68% of theor.) of the target compound.

LC-MS (method 10): Rₜ=1.23 min; MS (EIpos): m/z=340 [M+H]⁺.

¹H-NMR (400 MHz, DMSO-D₆): δ [ppm]=2.06 (s, 3H), 2.25 (s, 3H), 3.74 (s, 3H), 6.26 (s, 1H), 6.84-6.88 (m, 1H) 7.23-7.29 (m, 3H), 7.43-7.52 (m, 2H), 7.84 (dd, 1H), 9.34 (s, 1H).

### Example 146A

### Methyl-2-{[4-bromo-1-(2-fluoro-6-methylphenyl)-3-methyl-1H-pyrazol-5-yl]amino}benzoate

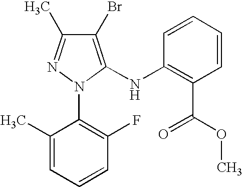

2.233 g (6.580 mmol) of the compound from example 145A was dissolved in 36 ml dichloromethane and, while cooling with ice, 0.941 g (3.290 mmol) of 1,3-dibromo-5,5-dimethylhydantoin was added in portions. Reaction testing by HPLC showed that reaction was completed after 20 min Dichloromethane was added and the organic phase was washed with water, saturated aqueous sodium hydrogen carbonate solution and 10% aqueous sodium thiosulfate solution. Then the organic phase was dried over sodium sulfate and concentrated in a rotary evaporator. We obtained 2.599 g (94% of theor.) of the target compound.

LC-MS (method 10): Rₜ=1.35 min; MS (EIpos): m/z=418 [M+H]⁺.

¹H-NMR (400 MHz, DMSO-D₆): δ [ppm]=2.11 (s, 3H), 2.28 (s, 3H), 3.80 (s, 3H), 6.59 (d, 1H), 6.80-6.84 (m, 1H) 7.19-7.25 (m, 2H), 7.38-7.45 (m, 2H), 7.80 (dd, 1H), 9.05 (s, 1H).

### Example 147A

### Methyl-2-{[4-(8-fluoroquinoxalin-6-yl)-1-(2-fluoro-6-methylphenyl)-3-methyl-1H-pyrazol-5-yl]amino}benzoate

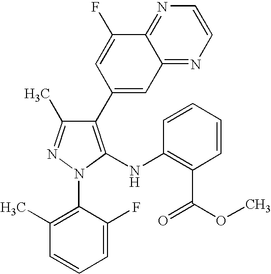

54 mg (0.060 mmol) of tris-(dibenzylidene-acetone)-dipalladium(0) and 40 mg (0.143 mmol) of tricyclohexylphosphine were added to 1092 mg of the raw product from example 91A. It was evacuated 5 times and ventilated with argon. Then 3 ml dioxane, 500 mg (1.195 mmol) of the compound from example 146A and 1.60 ml of 1.27 M aqueous potassium phosphate solution were added. It was stirred at 100° C. for 5 h. After cooling, water was added and it was extracted with ethyl acetate. The combined organic phases were dried over sodium sulfate and concentrated in a rotary evaporator. The residue was purified by preparative HPLC (eluent: acetonitrile/water, gradient 10:90→90:10). We obtained 317 mg (45% of theor., purity 83% according to HPLC) of the target compound.

LC-MS (method 10): Rₜ=1.21 min; MS (EIpos): m/z=486 [M+H]⁺.

### EXAMPLES OF APPLICATION

### Example 1

### 2-{[4-(Quinoxalin-6-yl)-3-methyl-1-(2-methylphenyl)-1H-pyrazol-5-yl]amino}-5-fluorobenzoic acid

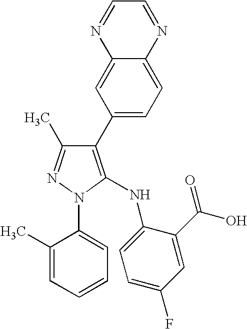

10 mg (0.021 mmol) of example 2A was dissolved in 1.25 ml dioxane/water (4/1) and 43 μl (0.043 mmol) of 2M sodium hydroxide solution was added. Then it was reacted overnight at room temperature. After completion of reaction had been detected by analytical HPLC, the volatile components were removed by distillation at reduced pressure. The residue was taken up in 20 ml water and the resultant solution was neutralized with saturated aqueous ammonium hydrochloride solution. Then it was extracted with 20 ml ethyl acetate (2×). Then the combined organic phases were dried over magnesium sulfate. The solvent was removed in a rotary evaporator and the product was finally dried under high vacuum. In this way we obtained 9.0 mg (88% of theor.) of the target compound.

LC-MS (method 3): Rₜ=1.89 min; MS (EIpos): m/z=454 [M+H]⁺.

¹H-NMR (400 MHz, DMSO-D₆): δ [ppm]=2.17 (s, 3H), 2.48 (s, 3H), 6.47 (dd, 1H), 7.03 (mc, 1H), 7.24 (m, 1H). 7.31-7.34 (m, 2H), 7.37 (dd, 1H), 7.39 (d, 1H), 7.97 (dd, 1H), 8.05 (d, 1H), 8.10 (d, 1H), 8.89 (d, 1H), 8.90 (d, 1H), 9.37 (sbr, 1H), 13.39 (sbr, 1H).

### Example 2

### 2-{[4-(Quinoxalin-6-yl)-3-methyl-1-(2-methylphenyl)-1H-pyrazol-5-yl]amino}-5-chlorobenzoic acid

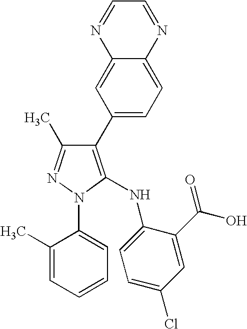

57 mg (0.118 mmol) of example 3A was dissolved in 2.5 ml dioxane/water (4/1) and 236 μl (0.236 mmol) of 1M sodium hydroxide solution was added. Then it was reacted overnight at room temperature. After completion of reaction had been detected by analytical HPLC, the volatile components were removed by distillation at reduced pressure. The residue was taken up in 20 ml water and the resultant solution was neutralized with saturated aqueous ammonium hydrochloride solution. Then it was extracted with 20 ml ethyl acetate (2×). Then the combined organic phases were dried over magnesium sulfate. The solvent was removed in a rotary evaporator and the product was finally dried under high vacuum. In this way we obtained 45 mg (81% of theor.) of the target compound.

LC-MS (method 1): Rₜ=2.45 min; MS (EIpos): m/z=470 [M+H]⁺.

¹H-NMR (400 MHz, DMSO-D₆): δ [ppm]=2.17 (s, 3H), 2.47 (s, 3H), 6.36 (d, 1H), 6.93 (d, 1H), 7.24 (m, 1H). 7.28-7.40 (m, 3H), 7.64 (sbr, 1H), 7.93 (d, 1H), 8.00 (d, 1H), 8.06 (d, 1H), 8.87 (d, 1H), 8.89 (d, 1H).

### Example 3

### 2-{[4-(Quinoxalin-6-yl)-3-methyl-1-(2-methylphenyl)-1H-pyrazol-5-yl]amino}-5-methylbenzoic acid

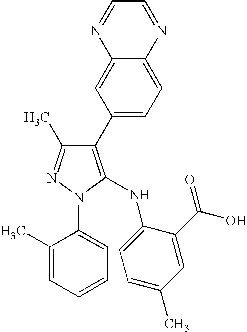

18 mg (0.038 mmol) of example 4A was dissolved in 1.25 ml dioxane/water (4/1) and 75 μl (0.075 mmol) of 1M sodium hydroxide solution was added. Then it was reacted overnight at room temperature. After completion of reaction had been detected by analytical HPLC, the volatile components were removed by distillation at reduced pressure. The residue was taken up in 20 ml water and the resultant solution was neutralized with saturated aqueous ammonium hydrochloride solution. Then it was extracted with 20 ml ethyl acetate (2×). Then the combined organic phases were dried over magnesium sulfate. The solvent was removed in a rotary evaporator and the product was finally dried under high vacuum. In this way we obtained 10 mg (59% of theor.) of the target compound.

LC-MS (method 2): Rₜ=2.19 min; MS (EIpos): m/z=450 [M+H]⁺.

¹H-NMR (400 MHz, DMSO-D₆): δ [ppm]=2.03 (s, 3H), 2.16 (s, 3H), 2.48 (s, 3H), 6.37 (d, 1H), 6.94 (m, 1H), 7.23 (m, 1H), 7.30-7.34 (m, 2H), 7.37 (d, 1H), 7.47 (d, 1H), 7.96 (dd, 1H), 8.03 (d, 1H), 8.09 (d, 1H), 8.88 (d, 1H), 8.90 (d, 1H), 12.97 (s, 1H).

### Example 4

### 2-{[4-(Quinoxalin-6-yl)-3-methyl-1-(2-methylphenyl)-1H-pyrazol-5-yl]amino}-benzoic acid

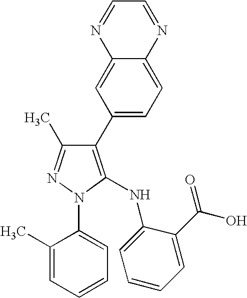

27 mg (0.060 mmol) of example 5A was dissolved in 1.5 ml dioxane/water (4/1) and 120 μl (0.120 mmol) of 1M sodium hydroxide solution was added. Then it was reacted overnight at room temperature. After completion of reaction had been detected by analytical HPLC, the volatile components were removed by distillation at reduced pressure. The residue was taken up in 20 ml water and the resultant solution was neutralized with saturated aqueous ammonium hydrochloride solution. Then it was extracted with 20 ml ethyl acetate (2×). Then the combined organic phases were dried over magnesium sulfate. The resultant raw product was purified by preparative HPLC (eluent: acetonitrile/water, gradient 10:90→90:10). This gave 15 mg (57% of theor.) of the target compound.

LC-MS (method 1): Rₜ=2.21 min; MS (EIpos): m/z=436 [M+H]⁺.

¹H-NMR (400 MHz, DMSO-D₆): δ [ppm]=2.17 (s, 3H), 2.48 (s, 3H), 6.46 (d, 1H), 6.57 (t, 1H), 7.10 (mc, 1H), 7.24 (mc, 1H), 7.29-7-35 (m, 2H), 7.40 (d, 1H), 7.66 (dd, 1H), 7.97 (dd, 1H), 8.04 (d, 1H), 8.10 (d, 1H), 8.88 (d, 1H), 8.89 (d, 1H), 9.58 (sbr, 1H), 13.04 (sbr, 1H).

### Example 5

### 2-{[4-(Quinoxalin-6-yl)-1-(2-chlorophenyl)-3-methyl-1H-pyrazol-5-yl]amino}-5-methoxybenzoic acid

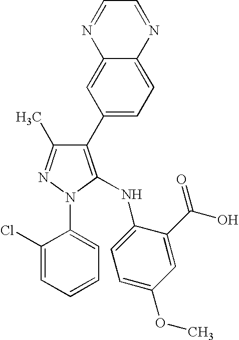

50 mg (0.100 mmol) of example 8A was dissolved in 4.0 ml dioxane/water (4/1) and 200 μl (0.200 mmol) of 1M sodium hydroxide solution was added. Then it was reacted overnight at room temperature. After completion of reaction had been detected by analytical HPLC, the volatile components were removed by distillation at reduced pressure. The residue was taken up in 20 ml water and the resultant solution was neutralized with 1N hydrochloric acid. Then it was extracted with 20 ml ethyl acetate (2×). Then the combined organic phases were dried over magnesium sulfate. The resultant product was dried under high vacuum. This gave 48 mg (99% of theor.) of the target compound.

LC-MS (method 1): Rₜ=2.20 min; MS (EIpos): m/z=486 [M+H]⁺.

¹H-NMR (400 MHz, DMSO-D₆): δ [ppm]=2.48 (s, 3H), 3.55 (s, 3H), 6.46 (d, 1H), 6.77 (dd, 1H), 7.15 (d, 1H), 7.41-7.58 (m, 2H), 7.60-7.67 (mc, 2H), 7.93 (dd, 1H), 8.04 (d, 1H), 8.07 (d, 1H), 8.89 (d, 1H), 8.90 (d, 1H), 9.31 (sbr, 1H), 13.20 (sbr, 1H).

### Example 6

### 2-{[4-(Quinoxalin-6-yl)-1-(2-methylphenyl)-3-(trifluoromethyl)-1H-pyrazol-5-yl]amino}-5-chlorobenzoic acid

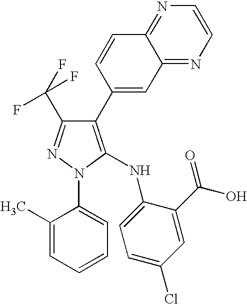

138 mg (0.209 mmol) of example 11A and 1.58 g (7.51 mmol) quinoxalin-6-yl boric acid hydrochloride were dissolved in 1.5 ml DMF. After adding 0.420 ml (8.00 mmol) of 2M aqueous sodium carbonate solution it was outgassed with argon. 21.7 mg (0.019 mmol) of tetrakis(triphenylphosphine)palladium(0) was added. Then was held at 110° C. for 4 h. By checking with LC-MS, in addition to the expected methyl ester we already detected a significant proportion of the corresponding acid. The mixture was filtered on kieselguhr. It was rewashed with dichloromethane and the filtrate was concentrated in a rotary evaporator. The organic phase was dried over sodium sulfate and the solvent was removed in a rotary evaporator. The resultant raw product was purified by preparative HPLC (eluent: acetonitrile/water, gradient 10:90→90:10). This gave 50 mg (46% of theor.) of the target compound.

LC-MS (method 1): Rₜ=2.79 min; MS (EIpos): m/z=524 [M+H]⁺.

¹H-NMR (400 MHz, DMSO-D₆): δ [ppm]=2.19 (s, 3H), 6.58 (d, 1H), 7.18 (dd, 1H), 7.30 (mc, 1H), 7.36-7.43 (m, 2H), 7.52-7.59 (m, 2H), 7.93 (dd, 1H), 8.12 (d, 1H), 8.19 (d, 1H), 8.94-8.96 (mc, 2H), 9.57 (sbr, 1H), 13.47 (sbr, 1H).

### Example 7

### 2-{[4-(Quinoxalin-6-yl)-1-(2-ethylphenyl)-3-methyl-1H-pyrazol-5-yl]amino}-5-methoxybenzoic acid

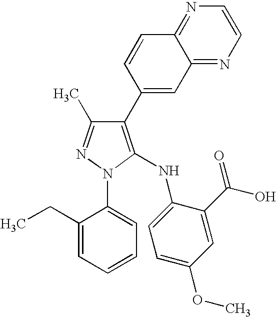

225 mg (0.456 mmol) of the compound from example 15A was dissolved in 20 ml dioxane/water (v/v=3:1) and 685 μl of 1N sodium hydroxide solution was added. It was stirred overnight at room temperature. The mixture was acidified with 1N hydrochloric acid and extracted with ethyl acetate. The combined organic phases were washed with a saturated aqueous sodium chloride solution, dried over sodium sulfate and concentrated in a rotary evaporator. The residue was dried under high vacuum. We obtained 220 mg (100% of theor.) of the target compound.

LC-MS (method 3): Rₜ=1.93 min; MS (EIpos): m/z=480 [M+H]⁺.

¹H-NMR (400 MHz, DMSO-D₆): δ [ppm]=1.07 (t, 3H), 2.47 (q, 2H), 2.48 (s, 3H), 3.55 (s, 3H), 6.45 (d, 1H), 6.76 (dd, 1H), 7.15 (d, 1H), 7.23-7.28 (m, 1H), 7.35 (d, 1H), 7.39 (d, 2H), 7.96 (dd, 1H), 8.02 (d, 1H), 8.09 (d, 1H), 8.88 (d, 1H), 8.89 (d, 1H), 9.23 (sbr, 1H), 13.15 (sbr, 1H).

### Example 8

### 5-Methoxy-2-{[3-methyl-1-(2-methylphenyl)-4-(pyrido[2,3-b]pyrazin-7-yl)-1H-pyrazol-5-yl]amino}benzoic acid

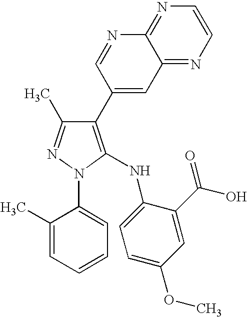

57 mg (0.119 mmol) of the compound from example 20A was dissolved in 5 ml dioxane/water (v/v=3:1) and 180 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was dried under high vacuum. We obtained 50 mg (90% of theor.) of the target compound.

LC-MS (method 1): Rₜ=2.00 min; MS (EIpos): m/z=467 [M+H]⁺.

¹H-NMR (400 MHz, DMSO-D₆): δ [ppm]=2.18 (s, 3H), 2.50 (s, 3H), 3.55 (s, 3H), 6.44 (d, 1H), 6.78 (dd, 1H), 7.16 (d, 1H), 7.23-7.29 (m, 1H), 7.32-7.35 (m, 2H), 7.39 (d, 1H), 8.51 (d, 1H), 9.02 (d, 1H), 9.07 (d, 1H), 9.25 (sbr, 1H), 9.26 (d, 1H), 13.18 (sbr, 1H).

### Example 9

### 2-{[4-(1H-Indazol-5-yl)-3-methyl-1-(2-methylphenyl)-1H-pyrazol-5-yl]amino}-5-methoxybenzoic acid

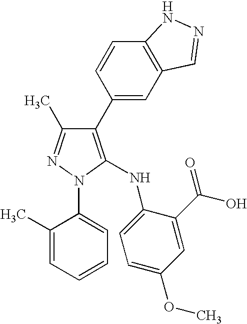

16 mg (0.034 mmol) of the compound from example 21A was dissolved in 1.25 ml dioxane/water (v/v=3:1) and 50 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was dried under high vacuum. We obtained 50 mg (90% of theor.) of the target compound.

LC-MS (method 1): Rₜ=2.13 min; MS (EIpos): m/z=4454 [M+H]⁺.

¹H-NMR (400 MHz, DMSO-D₆): δ [ppm]=2.14 (s, 3H), 2.36 (s, 3H), 3.56 (s, 3H), 6.40 (d, 1H), 6.77 (dd, 1H), 7.13 (d, 1H), 7.19-7.24 (m, 1H), 7.27-7.33 (m, 3H), 7.37 (dd, 1H), 7.46 (d, 1H), 7.75 (s, 1H), 8.10 (s, 1H), 9.09 (sbr, 1H) 13.02 (sbr, 1H), 13.06 (sbr, 1H).

### Example 10

### 2-{[4-(Quinolin-6-yl)-3-methyl-1-(2-methylphenyl)-1H-pyrazol-5-yl]amino}-5-methoxybenzoic acid

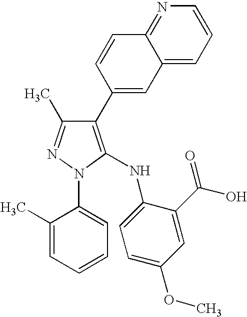

70 mg (0.146 mmol) of the compound from example 22A was dissolved in 5 ml dioxane/water (v/v=3:1) and 220 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was dried under high vacuum. We obtained 69 mg (100% of theor.) of the target compound.

LC-MS (method 3): Rₜ=1.60 min; MS (EIpos): m/z=465 [M+H]⁺.

¹H-NMR (400 MHz, DMSO-D₆): δ [ppm]=2.15 (s, 3H), 2.46 (s, 3H), 3.55 (s, 3H), 6.40 (d, 1H), 6.76 (dd, 1H), 7.14 (s, 1H), 7.22-7.27 (m, 1H), 7.30-7.39 (m, 3H), 7.66 (dd, 1H), 7.93 (dd, 1H), 8.00 (d, 1H), 8.10 (s, 1H), 8.49 (d, 1H), 8.95 (dd, 1H), 9.19 (s, 1H), 13.14 (sbr, 1H).

### Example 11

### 2-{[4-(1,3-Benzothiazol-5-yl)-3-methyl-1-(2-methylphenyl)-1H-pyrazol-5-yl]amino}-5-methoxybenzoic acid

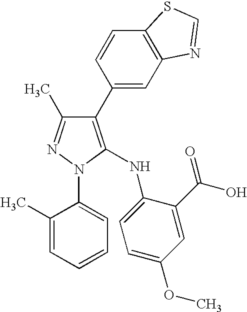

55 mg (0.114 mmol) of the compound from example 23A was dissolved in 2.7 ml dioxane/water (v/v=3:1) and 170 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was dried under high vacuum. We obtained 51 mg (95% of theor.) of the target compound.

LC-MS (method 4): Rₜ=1.23 min; MS (EIpos): m/z=471 [M+H]⁺.

¹H-NMR (400 MHz, DMSO-D₆): δ [ppm]=2.15 (s, 3H), 2.41 (s, 3H), 3.57 (s, 3H), 6.41 (d, 1H), 6.81 (dd, 1H), 7.14 (d, 1H), 7.20-7.25 (m, 1H), 7.29-7.32 (m, 2H), 7.35 (d, 1H), 7.54 (dd, 1H), 8.10 (s, 1H), 8.11 (d, 1H), 8.10 (s, 1H), 8.11 (d, 1H), 9.12 (sbr, 1H), 9.36 (s, 1H), 13.08 (sbr, 1H).

### Example 12

### 5-Methoxy-2-{[3-methyl-1-(2-methylphenyl)-4-(naphthalen-2-yl)-1H-pyrazol-5-yl]amino}benzoic acid

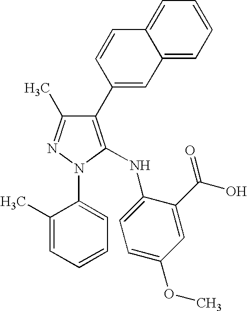

23 mg (0.048 mmol) of the compound from example 24A was dissolved in 2.2 ml dioxane/water (v/v=3:1) and 70 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was dried under high vacuum. We obtained 28 mg (100% of theor.) of the target compound.

LC-MS (method 2): Rₜ=2.51 min; MS (EIpos): m/z=464 [M+H]⁺.

¹H-NMR (400 MHz, DMSO-D₆): δ [ppm]=2.16 (s, 3H), 2.44 (s, 3H), 3.55 (s, 3H), 6.41 (d, 1H), 6.78 (dd, 1H), 7.14 (d, 1H), 7.22-7.26 (m, 1H), 7.29-7.34 (m, 2H), 7.36 (d, 1H), 7.44-7.50 (m, 2H), 7.57 (dd, 1H), 7.81-7.86 (m, 3H), 7.93 (s, 1H), 9.15 (sbr, 1H), 13.10 (sbr, 1H).

### Example 13

### 2-{[4-(Quinoxalin-6-yl)-3-methyl-1-(2-methylphenyl)-1H-pyrazol-5-yl]amino}-5-ethylbenzoic acid

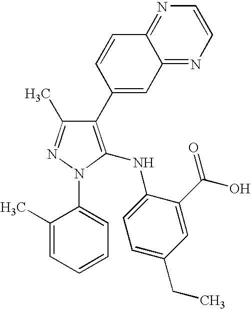

80 mg (0.168 mmol) of the compound from example 18A was dissolved in 8 ml dioxane/water (v/v=3:1) and 250 μl of 1N sodium hydroxide solution was added. It was stirred overnight at room temperature. A further 250 μl of 1N sodium hydroxide solution was added and the mixture was stirred overnight at 40° C. The reaction mixture was acidified with 1N hydrochloric acid and extracted with ethyl acetate. The combined organic phases were washed with saturated aqueous sodium chloride solution, dried over sodium sulfate and concentrated in a rotary evaporator. The residue was purified by preparative HPLC (eluent: acetonitrile/water, gradient 10:90→90:10). We obtained 58 mg (74% of theor.) of the target compound.

LC-MS (method 3): Rₜ=2.06 min; MS (EIpos): m/z=464 [M+H]⁺.

¹H-NMR (400 MHz, DMSO-D₆): δ [ppm]=0.97 (t, 3H), 2.17 (s, 3H), 2.34 (q, 2H), 2.48 (s, 3H), 6.39 (d, 1H), 6.97 (dd, 1H), 7.22-7.27 (m, 1H), 7.30-7.34 (m, 2H), 7.39 (d, 1H), 7.49 (d, 1H), 7.97 (dd, 1H), 8.04 (d, 1H), 8.10 (d, 1H), 8.88 (d, 1H), 8.89 (d, 1H), 9.43 (s, 1H), 12.99 (sbr, 1H).

### Example 14

### 2-{[4-(Quinoxalin-6-yl)-1-(2-methylphenyl)-1H-pyrazol-5-yl]amino}benzoic acid

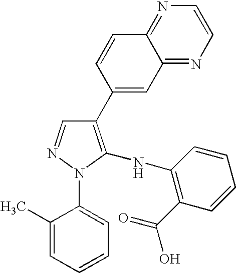

845 μl of 1N aqueous sodium hydroxide solution was added to 200 mg (0.42 mmol) of methyl-2-{[4-(quinoxalin-6-yl)-1-(2-methylphenyl)-1H-pyrazol-5-yl]amino}benzoate from example 27A in 8 ml dioxane/water (3:1). After stirring overnight at RT, the reaction mixture was acidified with 1N aqueous hydrochloric acid and separated by preparative HPLC (method 8). After concentration of the product fractions by evaporation, 163 mg (92% of theor.) of the target compound was obtained.

LC-MS (method 4): Rₜ=1.15 min; MS (EIpos): m/z=422 [M+H]⁺.

¹H NMR (400 MHz, DMSO-D₆): δ [ppm]=2.14 (s, 3H), 6.43 (d, 1H), 6.67 (t, 1H), 7.19 (td, 1H), 7.20-7.29 (m, 1H), 7.34 (d, 2H), 7.41 (d, 1H), 7.77 (dd, 1H), 8.05 (d, 1H), 8.19 (dd, 1H), 8.25 (s, 1H), 8.52 (s, 1H), 8.85 (AB-System, 2H), 9.63 (sbr, 1H), 13.13 (sbr, 1H).

### Example 15

### 2-{[4-(Quinoxalin-6-yl)-1-phenyl-1H-pyrazol-5-yl]amino}benzoic acid

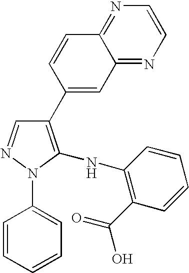

After reaction, processing and purification as in example 17, 80 mg (92% of theor.) of the target compound was obtained starting from 90 mg (0.21 mmol) of methyl-2-{[4-(quinoxalin-6-yl)-1-phenyl-1H-pyrazol-5-yl]amino}-benzoate from example 29A.

LC-MS (method 3): Rₜ=1.78 min; MS (EIpos): m/z=408 [M+H]⁺.

¹H NMR (400 MHz, DMSO-D₆): δ [ppm]=6.29 (d, 1H), 6.67 (t, 1H), 7.16 (td, 1H), 7.35 (t, 1H), 7.44 (t, 2H), 7.64 (d, 2H), 7.83 (dd, 1H), 8.05 (d, 1H), 8.20 (dd, 1H), 8.27 (s, 1H), 8.53 (s, 1H), 8.85 (AB-System, 2H), 9.77 (sbr, 1H), 13.25 (sbr, 1H).

### Example 16

### 2-{[4-(7-Fluoroquinoxalin-6-yl)-3-methyl-1-(2-methylphenyl)-1H-pyrazol-5-yl]amino}-5-methoxybenzoic acid

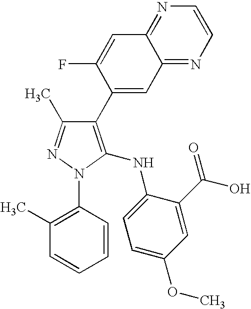

164 mg (approx. 0.132 mmol) of the compound from example 32A was dissolved in 2000 μl dioxane/water (v/v=3:1) and 200 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was purified by preparative HPLC (eluent: acetonitrile/water, gradient 10:90→90:10). We obtained 39 mg (61% of theor.) of the target compound.

LC-MS (method 3): Rₜ=2.29 min; MS (EIpos): m/z=484 [M+H]⁺.

¹H-NMR (400 MHz, DMSO-D₆): δ [ppm]=2.18 (s, 3H), 2.31 (s, 3H), 3.52 (s, 3H), 6.45 (d, 1H), 6.74 (dd, 1H), 7.08 (d, 1H), 7.23-7.36 (m, 3H), 7.39 (d, 1H), 7.90 (d, 1H), 8.15 (d, 1H), 8.90-8.93 (m, 2H) 9.16 (sbr, 1H), 13.12 (sbr, 1H).

### Example 17

### 2-{[4-(7-Chloroquinoxalin-6-yl)-3-methyl-1-(2-methylphenyl)-1H-pyrazol-5-yl]amino}-5-methoxybenzoic acid

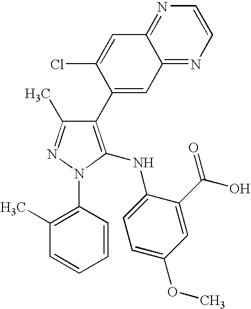

36 mg (approx. 0.069 mmol) of the compound from example 37A was dissolved in 3000 μl dioxane/water (v/v=3:1) and 100 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was purified by preparative HPLC (eluent: acetonitrile/water, gradient 10:90→90:10). We obtained 26 mg (74% of theor.) of the target compound.

LC-MS (method 4): Rₜ=1.22 min; MS (EIpos): m/z=500 [M+H]⁺.

¹H-NMR (400 MHz, DMSO-D₆): δ [ppm]=2.19 (s, 3H), 2.23 (s, 3H), 3.50 (s, 3H), 6.53 (d, 1H), 6.72 (dd, 1H), 7.04 (d, 1H), 7.24-7.29 (m, 1h), 7.32-7.38 (m, 3H), 8.18 (s, 1H), 8.24 (s, 1H), 8.95 (s, 2H), 9.10 (sbr, 1H), 13.08 (sbr, 1H).

### Example 18

### 5-Methoxy-2-{[3-methyl-4-(7-methylquinoxalin-6-yl)-1-(2-methylphenyl)-1H-pyrazol-5-yl]amino}benzoic acid

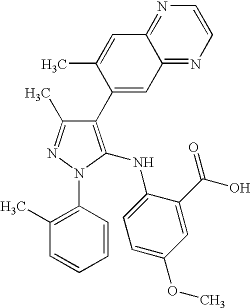

138 mg (purity 88%, 0.280 mmol) of the compound from example 42A was dissolved in 4000 μl dioxane/water (v/v=3:1) and 370 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was purified by preparative HPLC (eluent: acetonitrile/water, gradient 10:90→90:10). We obtained 96 mg (81% of theor.) of the target compound.

LC-MS (method 3): Rₜ=1.84 min; MS (EIpos): m/z=480 [M+H]⁺.

¹H-NMR (400 MHz, DMSO-D₆): δ [ppm]=2.18 (s, 3H), 2.19 (s, 3H), 2.43 (s, 3H), 3.51 (s, 3H), 6.54 (d, 1H), 6.75 (dd, 1H), 7.04 (d, 1H), 7.24-7.29 (m, 1h), 7.31-7.39 (m, 3H), 7.93 (s, 1H), 7.98 (s, 1H), 8.85 (d, 1H), 8.87 (d, 1H), 9.02 (sbr, 1H), 13.08 (sbr, 1H).

### Example 19

### 2-{[4-(5-Chloroquinoxalin-6-yl)-3-methyl-1-(2-methylphenyl)-1H-pyrazol-5-yl]amino}-5-methoxybenzoic acid

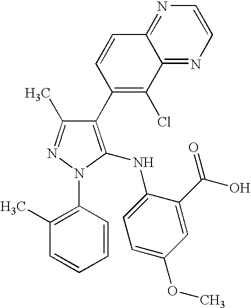

172 mg (approx. 0.167 mmol) of the compound from example 47A was dissolved in 2500 μl dioxane/water (v/v=3:1) and 250 μl of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was purified by preparative HPLC (eluent: acetonitrile/water, gradient 10:90→90:10). We obtained 66 mg (79% of theor.) of the target compound.

LC-MS (method 3): Rₜ=1.87 min; MS (EIpos): m/z=500 [M+H]⁺.

¹H-NMR (400 MHz, DMSO-D₆): δ [ppm]=2.19 (s, 3H), 2.23 (s, 3H), 3.48 (s, 3H), 6.49 (d, 1H), 6.68 (dd, 1H), 7.03 (d, 1H), 7.25-7.30 (m, 1h), 7.32-7.39 (m, 3H), 7.91 (d, 1H), 8.03 (d, 1H), 9.01 (d, 1H), 9.04 (d, 2H), 9.12 (sbr, 1H), 13.09 (sbr, 1H).

### Example 20

### 5-Methoxy-2-{[3-methyl-1-(2-methylphenyl)-4-([1,2,4]triazolo[1,5-a]pyridin-6-yl)-1H-pyrazol-5-yl]amino}benzoic acid

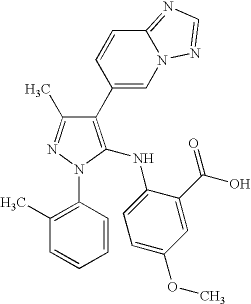

152 mg (0.324 mmol) of the compound from example 50A was dissolved in 4.8 ml dioxane/water (v/v=3:1) and 0.8 ml of 1N sodium hydroxide solution was added. It was stirred overnight at room temperature. The reaction mixture was acidified with 1N hydrochloric acid and extracted with ethyl acetate. The combined organic phases were washed with a saturated aqueous sodium chloride solution, dried over sodium sulfate and concentrated in a rotary evaporator. The residue was purified by preparative HPLC (eluent: acetonitrile/water, gradient 10:90→90:10). We obtained 116 mg (79% of theor.) of the target compound.

LC-MS (method 3): Rₜ=1.05 min; MS (EIpos): m/z=455 [M+H]⁺.

¹H-NMR (400 MHz, DMSO-D₆): δ [ppm]=2.15 (s, 3H), 2.41 (s, 3H), 3.57 (s, 3H), 6.41 (d, 1H), 6.81 (dd, 1H), 7.15 (d, 1H), 7.21-7.26 (m, 1H), 7.29-7.33 (m, 3H), 7.73 (dd, 1H), 7.81 (d, 1H), 8.47 (s, 1H), 9.00 (s, 1H), 9.11 (sbr, 1H), 13.10 (sbr, 1H).

### Example 21

### 2-{[4-(Quinoxalin-6-yl)-1-(2-methoxyphenyl)-3-methyl-1H-pyrazol-5-yl]amino}-5-methoxybenzoic acid

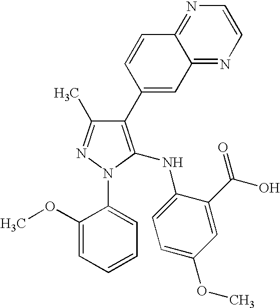

60 mg (0.121 mmol) of the compound from example 54A was dissolved in 1.8 ml dioxane/water (v/v=3:1) and 180 μl of 1N sodium hydroxide solution was added. It was stirred overnight at room temperature. The mixture was acidified with 1N hydrochloric acid and extracted with ethyl acetate. The combined organic phases were washed with a saturated aqueous sodium chloride solution, dried over sodium sulfate and concentrated in a rotary evaporator. The residue was dried under high vacuum. We obtained 57 mg (97% of theor.) of the target compound.

LC-MS (method 1): Rₜ=2.13 min; MS (EIpos): m/z=482 [M+H]⁺.

¹H-NMR (400 MHz, DMSO-D₆): δ [ppm]=2.46 (s, 3H), 3.52 (s, 3H), 3.86 (s, 3H), 6.40 (d, 1H), 6.68 (dd, 1H), 7.03 (t, 1H), 7.15 (d, 1H), 7.20 (d, 1H), 7.39-7.44 (m, 2H), 7.91 (dd, 1H), 8.00 (d, 1H), 8.04 (d, 1H), 8.87 (d, 1H), 8.89 (d, 1H) 9.37 (sbr, 1H), 13.22 (sbr, 1H).

### Example 22

### 2-{[4-(Quinoxalin-6-yl)-1-(2-ethoxyphenyl)-3-methyl-1H-pyrazol-5-yl]amino}-5-methoxybenzoic acid

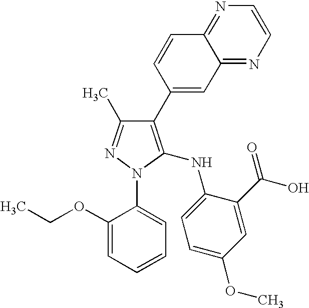

125 mg (0.343 mmol) of the compound from example 58A was dissolved in 5 ml dioxane/water (v/v=3:1) and 515 μl of 1N sodium hydroxide solution was added. It was stirred overnight at room temperature. The mixture was acidified with 1N hydrochloric acid and extracted with ethyl acetate. The combined organic phases were washed with a saturated aqueous sodium chloride solution, dried over sodium sulfate and concentrated in a rotary evaporator. The residue was purified by preparative HPLC (eluent: acetonitrile/water, gradient 10:90→90:10). We obtained 25 mg (15% of theor.) of the target compound.

LC-MS (method 4): Rₜ=1.14 min; MS (EIpos): m/z=496 [M+H]⁺.

¹H-NMR (400 MHz, DMSO-D₆): δ [ppm]=1.38 (t, 3H), 2.47 (s, 3H), 3.50 (s, 3H), 6.37 (d, 1H), 6.66 (dd, 1H), 7.02 (dt, 1H), 7.14 (d, 1H), 7.19 (d, 1H), 7.38-7.42 (m, 2H), 7.90 (dd, 1H), 8.01 (d, 1H), 8.03 (d, 1H), 8.87 (d, 1H), 8.88 (d, 1H) 9.39 (sbr, 1H), 13.22 (sbr, 1H).

### Example 23

### 2-{[4-(Quinoxalin-6-yl)-3-methyl-1-(2-methylphenyl)-1H-pyrazol-5-yl]amino}-5-(difluoromethoxy)benzoic acid

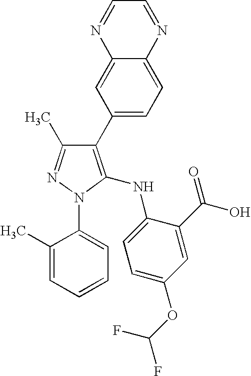

57 mg (0.111 mmol) of the compound from example 61A was dissolved in 2.2 ml dioxane/water (v/v=10/1) and 1.11 ml (1.11 mmol) of 1N sodium hydroxide solution was added. It was reacted overnight at room temperature. The solvents were removed in a rotary evaporator as far as possible. Then it was acidified with 1N hydrochloric acid and extracted with ethyl acetate. The combined organic phases were dried over magnesium sulfate and concentrated in a rotary evaporator. The residue was purified by preparative HPLC (eluent: acetonitrile/water, gradient 10:90→90:10). We obtained 50 mg (90% of theor.) of the target compound.

LC-MS (method 10): Rₜ=1.07 min; MS (EIpos): m/z=502 [M]⁺.

¹H-NMR (400 MHz, DMSO-D₆): δ [ppm]=2.18 (s, 3H), 2.48 (s, 3H), 6.49 (d, 1H), 6.94 (t, 1H), 7.00 (dd, 1H), 7.24 (m, 1H), 7.29-7.35 (m, 2H), 7.38-7.43 (m, 2H), 7.98 (dd, 1H), 8.05 (d, 1H), 8.11 (d, 1H), 8.89 (d, 1H), 8.90 (d, 1H), 9.48 (sbr, 1H), 13.40 (sbr, 1H).

### Example 24

### 2-{[4-(8-Fluoro-7-methoxyquinoxalin-6-yl)-3-methyl-1-(2-methylphenyl)-1H-pyrazol-5-yl]amino}-benzoic acid

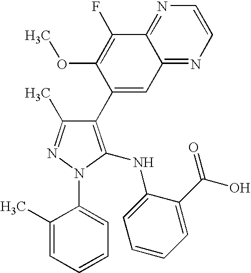

160 mg (approx. 0.161 mmol) of example 70A was dissolved in 8 ml dioxane/water (v/v=3/1) and 0.804 ml (0.804 mmol) of 1N sodium hydroxide solution was added. It was reacted overnight at room temperature. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. The residue was purified by preparative MPLC (Puriflash Analogix: 40S: isohexane/ethyl acetate=98/2→10/90). We obtained 25 mg (30% of theor.) of the target compound.

LC-MS (method 4): Rₜ=1.20 min; MS (EIpos): m/z=484 [M]⁺.

¹H-NMR (400 MHz, DMSO-D₆): δ [ppm]=2.20 (s, 3H), 2.28 (s, 3H), 4.01 (s, 3H), 6.48 (d, 1H), 6.53 (t, 1H), 7.10 (t, 1H), 7.24 (m, 1H), 7.27-7.35 (m, 2H), 7.39 (d, 1H), 7.58 (dd, 1H), 7.89 (d, 1H), 8.89 (d, 1H), 8.91 (d, 1H), 9.48 (sbr, 1H), 12.99 (sbr, 1H).

### Example 25

### 2-{[4-(8-Fluoro-7-methoxyquinoxalin-6-yl)-3-methyl-1-phenyl-1H-pyrazol-5-yl]amino}benzoic acid

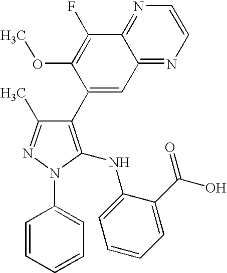

160 mg (approx. 0.199 mmol) of example 73A was dissolved in 8 ml dioxane/water (v/v=3/1) and 0.827 ml (0.827 mmol) of 1N sodium hydroxide solution was added. It was reacted overnight at room temperature. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. The residue was purified by preparative HPLC (eluent: acetonitrile/water, gradient 10:90→90:10). We obtained 50 mg (54% of theor.) of the target compound.

LC-MS (method 4): Rₜ=1.20 min; MS (EIpos): m/z=470 [M+H]⁺.

¹H-NMR (400 MHz, DMSO-D₆): δ [ppm]=2.28 (s, 3H), 4.03 (s, 1.5H), 4.04 (s, 1.5H), 6.34 (d, 1H), 6.56 (t, 1H), 7.11 (mc, 1H), 7.29 (t, 1H), 7.41 (t, 2H), 7.62-7.68 (m, 3H), 7.90 (d, 1H), 8.88 (d, 1H), 8.91 (d, 1H), 9.61 (sbr, 1H), 13.04 (sbr, 1H).

### Example 26

### 2-{[4-(3-Methoxyquinoxalin-6-yl)-3-methyl-1-(2-methylphenyl)-1H-pyrazol-5-yl]amino}benzoic acid

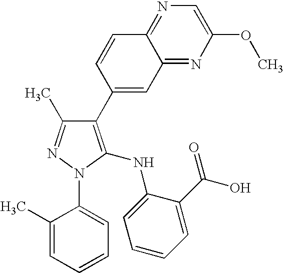

30 mg (approx. 0.050 mmol) of example 75A was dissolved in 5 ml dioxane and 0.626 ml (0.626 mmol) of 1N sodium hydroxide solution was added. It was reacted overnight at room temperature and then overnight at 80° C. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. The residue was purified by preparative HPLC (eluent: acetonitrile/water, gradient 10:90→90:10). We obtained 5 mg (22% of theor.) of the target compound.

LC-MS (method 10): Rₜ=1.15 min; MS (EIpos): m/z=466 [M+H]⁺.

¹H-NMR (400 MHz, DMSO-D₆): δ [ppm]=2.17 (s, 3H), 2.46 (s, 3H), 4.02 (s, 3H), 6.44 (d, 1H), 6.56 (t, 1H), 7.11 (t, 1H), 7.23 (m, 1H), 7.27-7.35 (m, 2H), 7.37 (d, 1H), 7.65 (dd, 1H), 7.71 (dd, 1H), 7.88 (d, 1H), 7.92 (d, 1H), 8.52 (s, 1H), 9.60 (sbr, 1H).

### Example 27

### 2-{[4-(Quinolin-7-yl)-3-methyl-1-(2-methylphenyl)-1H-pyrazol-5-yl]amino}benzoic acid

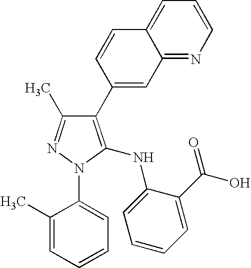

37 mg (0.082 mmol) of example 76A was dissolved in 5 ml dioxane/water (v/v=4/1) and 0.206 ml (0.206 mmol) of 1N sodium hydroxide solution was added. It was reacted overnight at room temperature and then overnight at 60° C. The mixture was concentrated by evaporation and taken up in water. Then it was acidified with 1N hydrochloric acid and extracted with ethyl acetate (2×20 ml). The combined organic phases were dried over magnesium sulfate, the volatile components were concentrated in a rotary evaporator and the resultant solid was dried under high vacuum. We obtained 25 mg (70% of theor.) of the target compound.

LC-MS (method 2): Rₜ=1.93 min; MS (EIpos): m/z=435 [M+H]⁺.

¹H-NMR (400 MHz, DMSO-D₆): δ [ppm]=2.17 (s, 3H), 2.47 (s, 3H), 6.46 (d, 1H), 6.57 (t, 1H), 7.12 (t, 1H), 7.23 (m, 1H), 7.28-7.35 (m, 2H), 7.39 (d, 1H), 7.50 (dd, 1H), 7.66 (dd, 1H), 7.72 (d, 1H), 7.94 (d, 1H), 8.04 (s, 1H), 8.33 (d, 1H), 8.87 (d, 1H), 9.53 (s, 1H), 13.02 (sbr, 1H).

### Example 28

### 2-({4-[3-(Dimethylamino)quinoxalin-6-yl]-3-methyl-1-(2-methylphenyl)-1H-pyrazol-5-yl}amino)benzoic acid

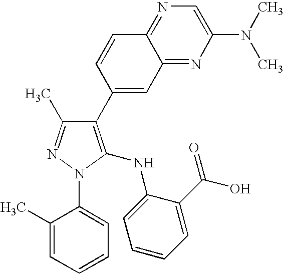

79 mg (0.160 mmol) of example 78A was dissolved in 5 ml dioxane and 1.604 ml (1.604 mmol) of 1N sodium hydroxide solution was added. It was reacted for 72 h at room temperature. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. The residue was purified by preparative HPLC (eluent: acetonitrile/water, gradient 10:90→90:10). We obtained 72 mg (89% of theor.) of the target compound.

LC-MS (method 10): Rₜ=1.09 min; MS (EIpos): m/z=479 [M+H]⁺.

¹H-NMR (400 MHz, DMSO-D₆): δ [ppm]=2.15 (s, 3H), 2.43 (s, 3H), 3.20 (s, 6H), 6.44 (d, 1H), 6.57 (t, 1H), 7.12 (t, 1H), 7.22 (mc, 1H), 7.27-7.33 (m, 2H), 7.36 (d, 1H), 7.42 (dd, 1H), 7.62 (d, 1H), 7.65 (d, 1H), 7.71 (d, 1H), 8.61 (s, 1H), 9.50 (sbr, 1H), 13.00 (sbr, 1H).

### Example 29

### 2-{[4-(8-Fluoro-7-hydroxyquinoxalin-6-yl)-3-methyl-1-(2-methylphenyl)-1H-pyrazol-5-yl]amino}benzoic acid

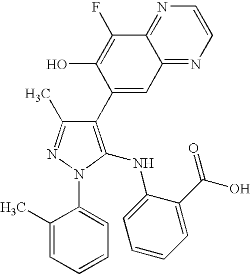

5 mg (0.010 mmol) of example 82A was dissolved in 0.6 ml dioxane/water (v/v=5/1) and 0.026 ml (0.026 mmol) of 1N sodium hydroxide solution was added. It was reacted overnight at room temperature and then overnight at 60° C. The mixture was concentrated by evaporation and taken up in water. Then it was acidified with 1N hydrochloric acid and extracted with ethyl acetate (2×20 ml). The combined organic phases were dried over magnesium sulfate and the volatile components were removed in a rotary evaporator. The residue was dried under high vacuum. We obtained 4 mg (82% of theor.) of the target compound.

LC-MS (method 2): Rₜ=1.97 min; MS (EIpos): m/z=470 [M+H]⁺.

¹H-NMR (400 MHz, DMSO-D₆): δ [ppm]=2.20 (s, 3H), 2.27 (s, 3H), 6.47-6.57 (m, 2H), 7.08 (t, 1H), 7.24 (mc, 1H), 7.27-7.34 (m, 2H), 7.38 (d, 1H), 7.58 (dd, 1H), 7.79 (d, 1H), 8.76 (d, 1H), 8.82 (d, 1H), 9.45 (sbr, 1H), 10.88 (sbr, 1H), 12.91 (sbr, 1H).

### Example 30

### 2-{[4-(7-Ethoxy-8-fluoroquinoxalin-6-yl)-3-methyl-1-(2-methylphenyl)-1H-pyrazol-5-yl]amino}benzoic acid

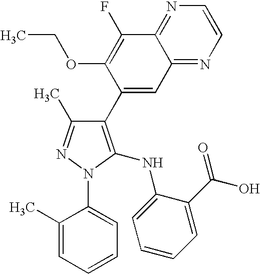

37 mg (0.072 mmol) of example 84A was dissolved in 4 ml dioxane/water (v/v=3/1) and 0.181 ml (0.181 mmol) of 1N sodium hydroxide solution was added. It was reacted overnight at room temperature and then overnight at 60° C. The mixture was concentrated by evaporation and taken up in water. Then it was acidified with 1N hydrochloric acid and extracted with ethyl acetate (2×20 ml). The combined organic phases were dried over magnesium sulfate and the volatile components were removed in a rotary evaporator. The residue was dried under high vacuum. We obtained 31 mg (84% of theor.) of the target compound.

LC-MS (method 2): Rₜ=2.28 min; MS (EIpos): m/z=498 [M+H]⁺.

¹H-NMR (400 MHz, DMSO-D₆): δ [ppm]=1.31 (t, 3H), 2.22 (s, 3H), 2.30 (s, 3H), 4.22 (q, 2H), 6.45 (d, 1H), 6.52 (t, 1H), 7.08 (t, 1H), 7.16-7.35 (m, 3H), 7.37 (d, 1H), 7.58 (d, 1H), 7.91 (s, 1H), 8.91 (d, 2H), 9.48 (s, 1H), 13.01 (sbr, 1H).

### Example 31

### 2-({4-[8-Fluoro-7-(2-hydroxyethoxy)quinoxalin-6-yl]-3-methyl-1-(2-methylphenyl)-1H-pyrazol-5-yl}amino)benzoic acid

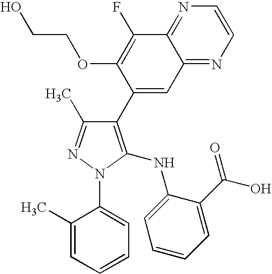

40 mg (0.070 mmol) of example 86A was dissolved in 4 ml dioxane/water (v/v=3/1) and 0.351 ml (0.351 mmol) of 1N sodium hydroxide solution was added. It was reacted overnight at room temperature. The mixture was concentrated by evaporation and taken up in water. Then it was acidified with 1N hydrochloric acid and extracted with ethyl acetate (2×20 ml). The combined organic phases were dried over magnesium sulfate and the volatile components were removed in a rotary evaporator. The residue was dried under high vacuum. We obtained 8.7 mg (24% of theor.) of the target compound.

LC-MS (method 2): Rₜ=1.99 min; MS (EIpos): m/z=514 [M+H]⁺.

¹H-NMR (400 MHz, DMSO-D₆): δ [ppm]=2.22 (s, 3H), 2.30 (s, 3H), 3.68 (t, 2H), 4.17 (t, 2H), 6.38-6.60 (m, 2H), 7.07 (m, 1H), 7.23 (dt, 1H), 7.28-7.35 (m, 2H), 7.39 (d, 1H), 7.57 (dd, 1H), 7.89 (s, 1H), 8.89 (d, 1H), 8.91 (d, 1H), 9.44 (sbr, 1H), 12.96 (sbr, 1H).

### Example 32

### 2-{[4-(4-Methoxyquinolin-6-yl)-3-methyl-1-(2-methylphenyl)-1H-pyrazol-5-yl]amino}benzoic acid

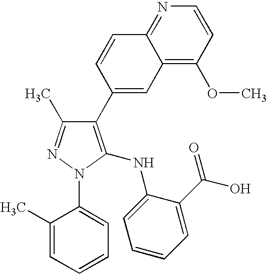

99 mg (0.207 mmol) of example 88A was dissolved in 12.5 ml dioxane/water (v/v=4/1) and 0.517 ml (0.517 mmol) of 1N sodium hydroxide solution was added. It was reacted overnight at room temperature. The mixture was concentrated by evaporation and taken up in water. Then it was acidified with 1N hydrochloric acid and the precipitated product was isolated by filtration. It was washed with a little water. Finally the solid was dried under high vacuum. We obtained 53 mg (55% of theor.) of the target compound.

LC-MS (method 2): Rₜ=1.73 min; MS (EIpos): m/z=465 [M+H]⁺.

¹H-NMR (400 MHz, DMSO-D₆): δ [ppm]=2.17 (s, 3H), 2.45 (s, 3H), 4.06 (s, 3H), 6.44 (d, 1H), 6.58 (t, 1H), 7.11 (mc, 1H), 7.16 (d, 1H), 7.25 (mc, 1H), 7.29-7.35 (m, 2H), 7.40 (d, 1H), 7.68 (dd, 1H), 7.95 (s, 2H), 8.17 (s, 1H), 8.82 (d, 1H), 9.49 (s, 1H), 13.03 (sbr, 1H).

### Example 33

### 2-{[4-(8-Fluoroquinoxalin-6-yl)-3-methyl-1-(2-methylphenyl)-1H-pyrazol-5-yl]amino}benzoic acid

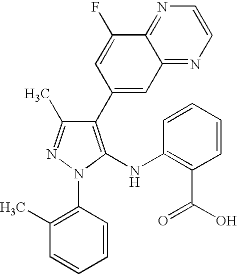

661 mg (1.174 mmol) of the compound from example 92A was dissolved in 17 ml dioxane/water (v/v=3:1) and 2.9 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 269 mg (50% of theor.) of the target compound.

LC-MS (method 10): Rₜ=1.06 min; MS (EIpos): m/z=454 [M+H]⁺.

¹H-NMR (400 MHz, DMSO-D₆): δ [ppm]=2.18 (s, 3H), 6.45 (d, 1H), 6.59 (t, 1H), 7.10-7.15 (m, 1H), 7.22-7.27 (m, 1H), 7.30-7.34 (m, 2H), 7.40 (d, 1H), 7.68 (dd, 1H), 7.84 (dd, 1H), 7.96 (sbr, 1H), 8.92 (d, 1H), 8.97 (d, 1H), 9.56 (sbr, 1H), 13.06 (sbr, 1H).

### Example 34

### 2-{[4-(8-Fluoroquinoxalin-6-yl)-3-methyl-1-phenyl-1H-pyrazol-5-yl]amino}benzoic acid

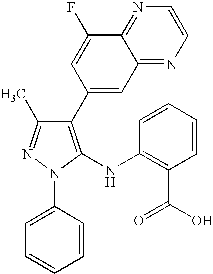

284 mg (0.519 mmol) of the compound from example 93A was dissolved in 8 ml dioxane/water (v/v=3:1) and 1.3 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 197 mg (86% of theor.) of the target compound.

LC-MS (method 10): Rₜ=1.05 min; MS (EIpos): m/z=440 [M+H]⁺.

¹H-NMR (400 MHz, DMSO-D₆): δ [ppm]=6.30 (d, 1H), 6.60 (t, 1H), 7.09-7.14 (m, 1H), 7.31 (t, 1H), 7.43 (t, 2H), 7.64 (d, 1H), 7.73 (dd, 1H), 7.85 (dd, 1H), 7.97 (sbr, 1H), 8.93 (d, 1H), 8.98 (d, 1H), 9.71 (sbr, 1H), 13.09 (sbr, 1H).

### Example 35

### 2-{[4-(7,8-Difluoroquinoxalin-6-yl)-3-methyl-1-(2-methylphenyl)-1H-pyrazol-5-yl]amino}benzoic acid

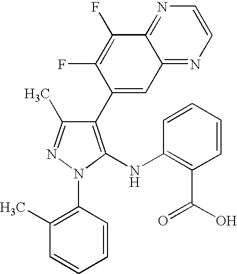

150 mg (0.117 mmol) of the compound from example 99A was dissolved in 4 ml dioxane/water (v/v=3:1) and 585 μl of 1N sodium hydroxide solution was added. The mixture was stirred overnight at room temperature. Then it was acidified with 1N hydrochloric acid. The mixture was extracted with ethyl acetate, the combined organic phases were washed with saturated sodium chloride solution and dried over sodium sulfate. The organic phase was concentrated in a rotary evaporator and the residue was purified by preparative HPLC (eluent: acetonitrile/water, gradient 10:90→90:10). We obtained 26 mg (45% of theor.) of the target compound.

LC-MS (method 4): Rₜ=1.25 min; MS (EIpos): m/z=472 [M+H]⁺.

¹H-NMR (400 MHz, DMSO-D₆): δ [ppm]=2.20 (s, 3H), 2.34 (s, 3H), 6.48 (d, 1H), 6.55 (t, 1H), 7.07-7.12 (m, 1H), 7.24-7.29 (m, 1H), 7.31-7.36 (m, 2H), 7.41 (d, 1H), 7.61 (dd, 1H), 8.05 (dd, 1H), 8.98-9.00 (m, 2H), 9.54 (sbr, 1H), 13.06 (sbr, 1H).

### Example 36

### 2-{[4-(7,8-Difluoroquinoxalin-6-yl)-3-methyl-1-phenyl-1H-pyrazol-5-yl]amino}benzoic acid

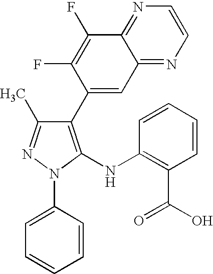

148 mg (0.261 mmol) of the compound from example 100A was dissolved in 4 ml dioxane/water (v/v=3:1) and 650 μl of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 69 mg (58% of theor.) of the target compound.

LC-MS (method 10): Rₜ=1.09 min; MS (EIpos): m/z=458 [M+H]⁺.

¹H-NMR (400 MHz, DMSO-D₆): δ [ppm]=2.34 (s, 3H), 6.33 (d, 1H), 6.57 (t, 1H), 7.08-7.13 (m, 1H), 7.32 (t, 1H), 7.43 (t, 2H), 7.64-7.68 (m, 3H), 8.05 (dd, 1H), 8.99 (s, 2H), 9.72 (sbr, 1H), 13.09 (sbr, 1H).

### Example 37

### 2-{[3-Methyl-1-(2-methylphenyl)-4-([1,2,4]triazolo[1,5-a]pyridin-7-yl)-1H-pyrazol-5-yl]amino}benzoic acid

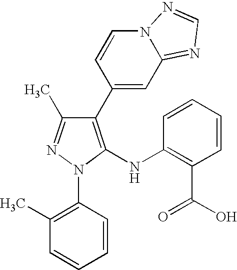

148 mg (0.261 mmol) of the compound from example 104A was dissolved in 3.2 ml dioxane/water (v/v=3:1) and 540 μl of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 57 mg (62% of theor.) of the target compound.

LC-MS (method 10): Rₜ=0.93 min; MS (EIpos): m/z=425 [M+H]⁺.

¹H-NMR (400 MHz, DMSO-D₆): δ [ppm]=2.15 (s, 3H), 2.47 (s, 3H), 6.44 (d, 1H), 6.61 (t, 1H), 7.13-7.17 (m, 1H), 7.21-7.25 (m, 1H), 7.28 (dd, 1H), 7.32 (d, 2H), 7.37 (d, 1H), 7.69 (dd, 1H), 7.84 (s, 1H), 8.43 (s, 1H), 8.87 (d, 1H), 9.54 (s, 1H), 13.06 (sbr, 1H).

### Example 38

### 2-{[4-(2-Amino [1,2,4]triazolo[1,5-a]pyridin-6-yl)-3-methyl-1-(2-methylphenyl)-1H-pyrazol-5-yl]amino}benzoic acid

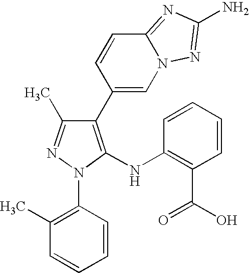

40 mg (0.088 mmol) of the compound from example 108A was dissolved in 1.5 ml dioxane/water (v/v=3:1) and 220 μl of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator. We obtained 40 mg (100% of theor.) of the target compound.

LC-MS (method 10): Rₜ=0.87 min; MS (EIpos): m/z=440 [M+H]⁺.

¹H-NMR (400 MHz, DMSO-D₆): δ [ppm]=2.16 (s, 3H), 2.38 (s, 3H), 6.09 (sbr, 2H), 6.44 (d, 1H), 6.60 (t, 1H), 7.14-7.19 (m, 1H), 7.20-7.25 (m, 1H), 7.28-7.35 (m, 4H), 7.52 (dd, 1H), 7.67 (dd, 1H), 8.59 (s, 1H), 9.41 (s, 1H), 12.99 (sbr, 1H).

### Example 39

### 2-{[4-(2-Amino [1,2,4]triazolo[1,5-a]pyridin-7-yl)-3-methyl-1-(2-methylphenyl)-1H-pyrazol-5-yl]amino}benzoic acid

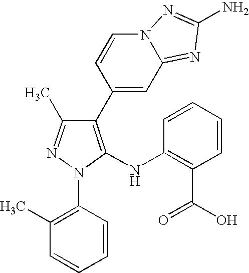

125 mg (0.276 mmol) of the compound from example 112A was dissolved in 4 ml dioxane/water (v/v=3:1) and 690 μl of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 66 mg (54% of theor.) of the target compound.

LC-MS (method 4): Rₜ=0.99 min; MS (EIpos): m/z=440 [M+H]⁺.

¹H-NMR (400 MHz, DMSO-D₆): δ [ppm]=2.14 (s, 3H), 2.43 (s, 3H), 5.93 (sbr, 2H), 6.43 (d, 1H), 6.62 (t, 1H), 6.92 (dd, 1H), 7.15-7.19 (m, 1H), 7.20-7.26 (m, 1H), 7.28-7.37 (m, 4H), 7.69 (dd, 1H), 8.44 (d, 1H), 9.48 (s, 1H), 13.04 (sbr, 1H).

### Example 40

### 2-{[4-(1-Aminoisoquinolin-7-yl)-3-methyl-1-(2-methylphenyl)-1H-pyrazol-5-yl]amino}benzoic acid

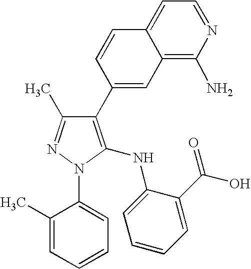

100 mg (0.216 mmol) of example 113A was dissolved in 7 ml dioxane and 2.157 ml (2.157 mmol) of 1N sodium hydroxide solution was added. It was reacted overnight. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. Finally it was dried under high vacuum. We obtained 64 mg (66% of theor.) of the target compound.

LC-MS (method 10): Rₜ=0.83 min; MS (EIpos): m/z=450 [M+H]⁺.

¹H-NMR (400 MHz, DMSO-D₆): δ [ppm]=2.17 (s, 3H), 2.44 (s, 3H), 6.37 (d, 1H), 6.56 (t, 1H), 7.05 (d, 1H), 7.09 (t, 1H), 7.25 (mc, 1H), 7.28-7.39 (m, 3H), 7.62-7.71 (m, 2H), 7.78 (d, 1H), 7.84 (d, 1H), 8.23 (sbr, 2H), 8.46 (s, 1H), 9.56 (s, 1H).

### Example 41

### 2-{[4-(8-Fluoroquinolin-6-yl)-3-methyl-1-phenyl-1H-pyrazol-5-yl]amino}benzoic acid

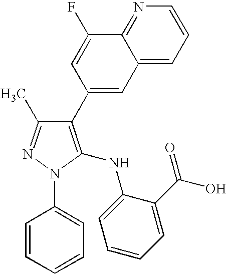

43 mg (0.095 mmol) of example 115A was dissolved in 5 ml dioxane/water (4/1) and 38 mg (0.950 mmol) sodium hydroxide was added. It was reacted overnight at a temperature 80° C. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. The residue was purified by preparative HPLC (eluent: acetonitrile/water, gradient 10:90→90:10). We obtained 17 mg (41% of theor.) of the target compound.

LC-MS (method 10): Rₜ=1.06 min; MS (EIpos): m/z=439 [M+H]⁺.

¹H-NMR (400 MHz, DMSO-D₆): δ [ppm]=2.48 (s, 3H), 6.28 (d, 1H), 6.59 (t, 1H), 7.11 (t, 1H), 7.31 (t, 1H), 7.43 (t, 2H), 7.56-7.77 (m, 5H), 7.86 (s, 1H), 8.34 (d, 1H), 8.89 (d, 1H), 9.68 (s, 1H), 13.11 (sbr, 1H).

### Example 42

### 2-{[4-(4-Methoxyquinolin-7-yl)-3-methyl-1-(2-methylphenyl)-1H-pyrazol-5-yl]amino}benzoic acid

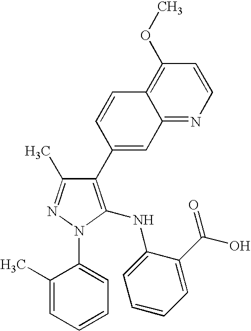

120 mg (0.251 mmol) of example 117A was dissolved in 5 ml dioxane/water (4/1) and 25 mg (0.627 mmol) sodium hydroxide was added. It was reacted overnight at room temperature. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. We obtained 87 mg (75% of theor.) of the target compound.

LC-MS (method 10): Rₜ=0.87 min; MS (EIpos): m/z=465 [M+H]⁺.

¹H-NMR (400 MHz, DMSO-D₆): δ [ppm]=2.16 (s, 3H), 2.47 (s, 3H), 4.11 (s, 3H), 6.44 (d, 1H), 6.58 (t, 1H), 7.11 (t, 1H), 7.16 (sbr, 1H), 7.24 (m, 1H), 7.29-7.35 (m, 2H), 7.38 (d, 1H), 7.67 (d, 1H), 7.77 (d, 1H), 8.04 (s, 1H), 8.13 (d, 1H), 8.84 (sbr, 1H), 9.56 (s, 1H).

### Example 43

### 2-{[4-(5-Fluoroquinazolin-7-yl)-3-methyl-1-(2-methylphenyl)-1H-pyrazol-5-yl]amino}benzoic acid

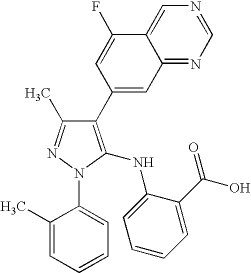

152 mg (0.325 mmol) of example 119A was dissolved in 5 ml dioxane and 3.25 ml (3.25 mmol) of 1N sodium hydroxide solution was added. It was reacted overnight at room temperature. The mixture was concentrated by evaporation and taken up in water. Then it was acidified with 1N hydrochloric acid and extracted with ethyl acetate (2×20 ml). The combined organic phases were dried over magnesium sulfate and concentrated in a rotary evaporator. We obtained 147 mg (99% of theor.) of the target compound.

LC-MS (method 10): Rₜ=1.05 min; MS (EIpos): m/z=454 [M+H]⁺.

¹H-NMR (400 MHz, DMSO-D₆): δ [ppm]=2.17 (s, 3H), 3.17 (s, 3H), 6.43 (d, 1H), 6.59 (t, 1H), 7.13 (t, 1H), 7.25 (m, 1H), 7.29-7.36 (m, 2H), 7.40 (d, 1H), 7.64-7.76 (2xed, 2H), 7.90 (s, 1H), 9.31 (s, 1H), 9.58 (s, 1H), 9.63 (s, 1H), 13.08 (sbr, 1H).

### Example 44

### 2-{[4-(8-Fluoroquinoxalin-6-yl)-1-phenyl-3-(trifluoromethyl)-1H-pyrazol-5-yl]amino}benzoic acid

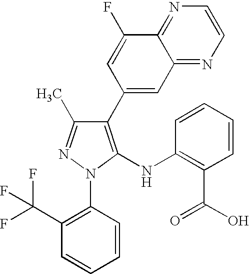

661 mg (1.174 mmol) of the compound from example 123A was dissolved in 11 ml dioxane/water (v/v=3:1) and 1.53 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 108 mg (27% of theor.) of the target compound.

LC-MS (method 10): Rₜ=1.07 min; MS (EIpos): m/z=508 [M+H]⁺.

¹H-NMR (400 MHz, DMSO-D₆): δ [ppm]=2.49 (s, 3H), 6.47 (d, 1H), 6.59 (t, 1H), 7.10 (dt, 1H), 7.66-7.80 (m, 5H), 7.90-7.94 (m, 2H), 8.92 (d, 1H), 8.97 (dd, 1H), 9.64 (sbr, 1H), 13.13 (sbr, 1H).

### Example 45

### 2-{[1-(2,5-Dimethylphenyl)-4-(8-fluoroquinoxalin-6-yl)-3-methyl-1H-pyrazol-5-yl]amino}benzoic acid

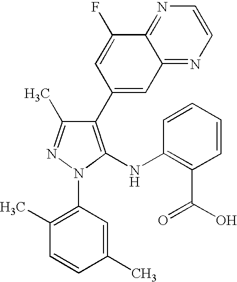

395 mg (0.821 mmol) of the compound from example 127A was dissolved in 20 ml dioxane/water (v/v=3:1) and 1.64 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 164 mg (43% of theor.) of the target compound.

LC-MS (method 10): Rₜ=1.11 min; MS (EIpos): m/z=468 [M+H]⁺.

¹H-NMR (400 MHz, DMSO-D₆): δ [ppm]=2.11 (s, 3H), 2.25 (s, 3H), 2.49 (s, 3H), 6.43 (d, 1H), 6.58 (t, 1H), 7.09-7.23 (m, 4H), 7.67 (dd, 1H), 7.84 (dd, 1H), 7.96 (s, 1H), 8.92 (d, 1H), 8.97 (dd, 1H), 9.57 (sbr, 1H), 13.09 (sbr, 1H).

### Example 46

### 2-{[1-(2,4-Difluorophenyl)-4-(8-fluoroquinoxalin-6-yl)-3-methyl-1H-pyrazol-5-yl]amino}benzoic acid

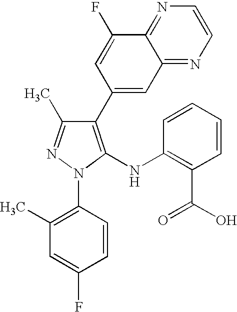

395 mg (0.821 mmol) of the compound from example 131A was dissolved in 25 ml dioxane/water (v/v=3:1) and 1.85 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 219 mg (50% of theor.) of the target compound.

LC-MS (method 10): Rₜ=1.06 min; MS (EIpos): m/z=476 [M+H]⁺.

¹H-NMR (400 MHz, DMSO-D₆): δ [ppm]=2.49 (s, 3H), 6.40 (d, 1H), 6.59-6.63 (m, 1H), 7.10-7.14 (m, 1H), 7.20-7.24 (m, 1H), 7.46-7.51 (m, 1H), 7.69-7.82 (m, 3H), 7.94 (s, 1H), 8.93 (d, 1H), 8.98 (d, 1H), 9.64 (sbr, 1H), 13.14 (sbr, 1H).

### Example 47

### 2-{[4-(8-Fluoroquinoxalin-6-yl)-1-(3-fluoro-2-methylphenyl)-3-methyl-1H-pyrazol-5-yl]amino}benzoic acid

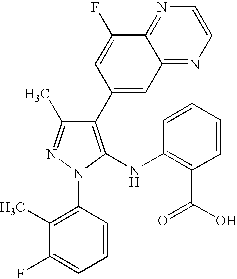

277 mg (0.570 mmol) of the compound from example 135A was dissolved in 15 ml dioxane/water (v/v=3:1) and 1.14 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 145 mg (54% of theor.) of the target compound.

LC-MS (method 10): Rₜ=1.08 min; MS (EIpos): m/z=472 [M+H]⁺.

¹H-NMR (400 MHz, DMSO-D₆): δ [ppm]=2.10 (d, 3H), 6.42 (d, 1H), 6.80 (t, 1H), 7.11-7.15 (m, 1H), 7.23-7.31 (m, 3H), 7.68 (dd, 1H), 7.86 (dd, 1H), 7.98 (s, 1H), 8.93 (d, 1H), 8.98 (d, 1H), 9.58 (sbr, 1H), 13.10 (sbr, 1H).

### Example 48

### 2-{[1-(2,5-Difluorophenyl)-4-(8-fluoroquinoxalin-6-yl)-3-methyl-1H-pyrazol-5-yl]amino}benzoic acid

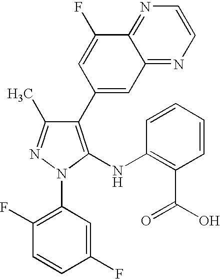

390 mg (0.797 mmol) of the compound from example 139A was dissolved in 20 ml dioxane/water (v/v=3:1) and 1.59 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 234 mg (62% of theor.) of the target compound.

LC-MS (method 10): Rₜ=1.07 min; MS (EIpos): m/z=476 [M+H]⁺.

¹H-NMR (400 MHz, DMSO-D₆): δ [ppm]=2.49 (s, 3H), 6.39 (d, 1H), 6.59-6.63 (m, 1H), 7.09-7.14 (m, 1H), 7.32-7.38 (m, 1H), 7.63-7.68 (m, 1H), 7.70 (dd, 1H), 7.81 (dd, 1H), 7.95 (s, 1H), 8.93 (d, 1H), 8.98 (d, 1H), 9.70 (sbr, 1H), 13.15 (sbr, 1H).

### Example 49

### 2-{[4-(8-Fluoroquinoxalin-6-yl)-1-(4-fluoro-2-methylphenyl)-3-methyl-1H-pyrazol-5-yl]amino}benzoic acid

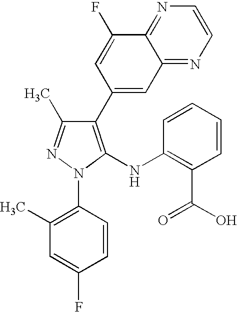

428 mg (0.882 mmol) of the compound from example 143A was dissolved in 13 ml dioxane/water (v/v=3:1) and 1.76 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 194 mg (47% of theor.) of the target compound.

LC-MS (method 4): Rₜ=1.22 min; MS (EIpos): m/z=472 [M+H]⁺.

¹H-NMR (400 MHz, DMSO-D₆): δ [ppm]=2.17 (s, 3H), 6.80 (dt, 1H), 7.07-7.15 (m, 2H), 7.21 (dd, 1H), 7.48 (dd, 1H), 7.69 (dd, 1H), 7.84 (dd, 1H), 7.96 (s, 1H), 8.92 (d, 1H), 8.98 (d, 1H), 9.55 (sbr, 1H), 13.08 (sbr, 1H).

### Example 50

### 2-{[4-(8-Fluoroquinoxalin-6-yl)-1-(2-fluoro-6-methylphenyl)-3-methyl-1H-pyrazol-5-yl]amino}benzoic acid

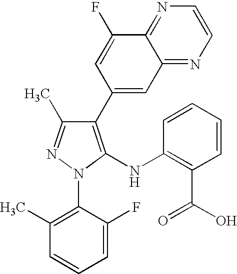

316 mg (0.542 mmol) of the compound from example 147A was dissolved in 13 ml dioxane/water (v/v=3:1) and 1.76 ml of 1N sodium hydroxide solution was added. The mixture was stirred overnight at room temperature. Then it was acidified with 1N hydrochloric acid. The mixture was extracted with ethyl acetate, the combined organic phases were washed with saturated aqueous sodium chloride solution and dried over sodium sulfate. The organic phase was concentrated in a rotary evaporator and the residue was purified by preparative HPLC (eluent: acetonitrile/water, gradient 10:90→90:10). We obtained 101 mg (39% of theor.) of the target compound.

LC-MS (method 10): Rₜ=1.07 min; MS (EIpos): m/z=472 [M+H]⁺.

¹H-NMR (400 MHz, DMSO-D₆): δ [ppm]=2.15 (s, 3H), 2.48 (s, 3H), 6.26 (d, 1H), 6.71 (t, 1H), 7.19 (t, 2H), 7.38 (dt, 1H), 7.65-7.71 (m, 2H), 7.89 (s, 1H), 8.88 (d, 1H), 8.94 (d, 1H), 12.87 (sbr, 1H).

### B. ASSESSMENT OF PHARMACOLOGICAL EFFICACY

The pharmacological effects of the compounds according to the invention can be shown in the following assays:

### Abbreviations:

EDTA Ethylenediaminetetraacetic acid
DMEM Dulbecco modified Eagle medium
FCS Fetal calf serum
HEPES 4-(2-hydroxyethyl)-1-piperazine-ethanesulfonic acid
SmGM Smooth muscle cell growth media
Tris-HCl 2-Amino-2-(hydroxymethyl)-1,3-propanediol hydrochloride
UtSMC Uterine smooth muscle cells

### B-1. Indirect Determination of Adenosine Antagonism Via Gene Expression

Cells of the permanent line CHO K₁ (Chinese Hamster Ovary) are stably transfected with a reporter construct (CRE luciferase) and the cDNA for the adenosine receptor subtypes A2a or A2b. A2a or A2b receptors are coupled via Gαs proteins to adenylate cyclase. Through receptor activation, the adenylate cyclase is activated and therefore the cAMP level in the cell is increased. Via the reporter construct, a cAMP-dependent promoter, the change in the cAMP level is coupled to luciferase expression.

For determination of adenosine antagonism on the adenosine receptor subtype A1, once again CHO K₁ cells are stably transfected, but this time with a Ca²⁺-sensitive reporter construct (NFAT-TA-Luc; Clontech) and an A1-Gα16 fusion construct. This receptor chimera is, in contrast to the native A1 receptor (Gαi-coupling), coupled to phospholipase C. The luciferase is expressed here as a function of the cytosolic Ca²⁺ concentration.

The permanent cell lines are cultured in DMEM/F12-Glutamax (Cat.No. 31331-028; Gibco) with 10% FCS (fetal calf serum) and various additives (10 ml/liter 1M HEPES (Cat.No. 15630; Gibco), 14 ml/liter MEM sodium pyruvate (Cat.No. 11360-039; Gibco) at 37° C. under 5% carbon dioxide, and split twice weekly.

For testing in the 384-well or 96-well plate format, the cells are sown at 2000 or 5000 cells/well in 25 or 50 μl/well test medium (OptiMEM-Glutamax with 2.5% activated-charcoal-treated FCS, Hyclone) and cultured at 37° C. under 5% carbon dioxide until substance testing. The A2a and A2b cells are sown, 24 h before substance testing, in OptiMEM with 2.5% activated-charcoal-treated FCS (Hyclone). The A1-Gα16 cells are sown, 48 h before substance testing, in OptiMEM-Glutamax with 2.5% activated-charcoal-treated FCS and additives. The substances are pipetted at a final concentration from 1×10⁻⁵ M to 1×10⁻¹¹ M to the test cultures, with the DMSO content on the cells not exceeding 0.5%. NECA (5-N-ethyl-carboxamido-adenosine) at a final concentration of 30 nM, which roughly corresponds to the EC₅₀ concentration, is used as agonist for the A2a and A2b cells. 25 nM CPA (N6-cyclopenty adenosine), which roughly corresponds to the EC₇₋₅ concentration, is used as agonist for the A1-Ga16 cells. After adding the substances, the cell plates are incubated for 3-4 h at 37° C. under 5% carbon dioxide. Then 50 μl of a solution consisting to 50% of lysis reagent (Triton buffer, PAA Cat.No. T21-160) and to 50% of luciferase substrate solution (2.5 mM ATP, 0.5 mM luciferin, 0.1 mM coenzyme A, 10 mM Tricin, 1.35 mM magnesium sulfate, 15 mM DTT, pH 7.8) is added to the cells directly before measurement. The luciferase activity is detected with a luminescence reader. The IC₅₀ values are determined, i.e. the concentrations at which the luciferase response, produced by the respective agonist, is inhibited to 50%. The IC₅₀ values are calculated with the computer program GraphPad PRISM (Version 3.02). ZM241385, for the A2a and A2b cells, and DPCPX (1,3-dipropyl-8-cyclopentylxanthine), for the A1-Gα16 cells, are used as reference antagonist.

| Example | IC50 A1 | IC50 A2a | IC50 A2b |
|---|---|---|---|
| No. | [nM] | [nM] | [nM] |
| 2 | 1 | >10000 | >10000 |
| 6 | 9.5 | >10000 | >10000 |
| 9 | 11.2 | >10000 | >10000 |
| 11 | 11.7 | >10000 | >10000 |
| 13 | 1 | >10000 | >10000 |
| 16 | 1.6 | >10000 | >10000 |
| 20 | 1.9 | >10000 | >10000 |
| 22 | 1 | >10000 | >10000 |
| 27 | 99 | >10000 | >10000 |
| 28 | 19 | | |
| 31 | 42 | | |
| 32 | 78 | | |
| 33 | 0.6 | >10000 | >10000 |
| 36 | 0.7 | >10000 | >10000 |
| 39 | 1 | >10000 | >10000 |
| 43 | 23 | >10000 | >10000 |
| 46 | 8.5 | >10000 | >10000 |

B-2. Binding Studies on Membrane Preparations of Cells with Adenosine A1 Receptors

For the production of cell membranes with human adenosine A1 receptors, CHO cells stably overexpressing A1 receptors (see B1) were lysed and then centrifuged differentially. After lysis in binding buffer ((50 mM Tris-(hydroxymethyl)-aminomethane/1 N hydrochloric acid, 5 mM magnesium chloride, pH 7.4 with an Ultra Turrax (Jahnke&Kunkel, Ika-Werk), the homogenate is centrifuged at 1000 g at 4° C. for 10 min. The resultant sediment is discarded and the supernatant is centrifuged at 20000 g at 4° C. for 30 mM The supernatant is discarded and the sediment is resuspended in binding buffer and stored at −70° C. until the binding test.

For the binding test, 1 nM ³H-DPCPX (1,3-dipropyl-8-cyclopentylxanthine) (4.44 TBq/mmol, PerkinElmer) is incubated for 60 minutes with 30-300 μg/ml human cell membranes in binding buffer with 0.2 units/ml adenosine deaminase added (Sigma) (total test volume 0.2 ml) in the presence of the test substances at 30° C. in 96-well filter plates (FC/B glass fiber, Multiscreen Millipore). After ending the incubation by removing the unbound radioactivity by suction, the plates are washed with binding buffer to which 0.1% bovine serum albumin has been added, and then dried overnight at 40° C. Then liquid scintillator (Ultima Gold, PerkinElmer) is added and the radioactivity still on the plates is measured in a liquid scintillation counter (Microbeta, Wallac). The nonspecific binding is defined as radioactivity in the presence of 1 μM DPCPX (Sigma) and is as a rule <25% of the total bound radioactivity. The binding data (IC50 and dissociation of constant Ki) are determined by means of the program GraphPad Prism Version 4.0.

### B-3. In Vivo Test for Detecting Cardiovascular Effects: Measurement of Blood Pressure of Anesthetized Rats

Male Wistar rats with a body weight of 300-350 g are anesthetized with thiopental (100 mg/kg i.p.). After tracheotomy, a catheter for measurement of blood pressure is inserted in the femoral artery. The test substances are administered as solutions either orally by stomach tube or intravenously via the femoral vein (Stasch et al. Br. J. Pharmacol. 2002; 135: 344-355).

### B-4. In Vivo Test for Detecting Cardiovascular Effects: Measurements of Blood Pressure on Conscious Spontaneously Hypertensive Rats

The measurement of blood pressure on conscious rats described below employs a commercially available telemetry system from the company DATA SCIENCES INTERNATIONAL DSI, USA.

The system consists of 3 main components:
1. Implantable transmitter (Physiotel® telemetry transmitter)
2. Receiver (Physiotel® receiver), which are connected via a multiplexer (DSI Data Exchange Matrix) to a
3. Data acquisition computer.

The telemetry equipment provides continuous recording of blood pressure, heart rate and body movement of conscious animals in their familiar environment.

### Animal Material

The investigations are carried out on adult, female, spontaneously hypertensive rats (SHR Okamoto) with a body weight of >200 g. SHR/NCrl crossed by Okamoto Kyoto School of Medicine, 1963 from male Wistar Kyoto rats with greatly increased blood pressure and females with slightly increased blood pressure and deposited in F13 with the U.S. National Institutes of Health.

After transmitter implantation, the test animals are kept individually in Type 3 Macrolon cages. They have free access to standard feed and water.

The day-night rhythm in the test laboratory is changed by room lighting at 6:00 h in the morning and at 19:00 h in the evening.

### Transmitter Implantation

The telemetry transmitters used, TA11 PA-C40, are surgically implanted under aseptic conditions in the test animals at least 14 days before they are used in the first test. After the wound has healed and the implant has grown in, the instrumented animals can be used repeatedly.

For implantation, the fasting animals are anesthetized with pentobarbital (Nembutal, Sanofi: 50 mg/kg i.p.) and are shaved and disinfected over a wide area of the ventral side. After opening the abdominal cavity along the linea alba, the liquid-filled measuring catheter of the system is inserted above the bifurcation to cranial in the aorta descendens and secured with tissue adhesive (VetBonD™, 3M). The transmitter housing is fixed intraperitoneally to the musculature of the abdominal wall and the wound is closed layer by layer.

Postoperatively, an antibiotic is administered as prophylaxis against infection (Tardomyocel Comp Bayer 1 ml/kg s.c.)

### Substances and Solutions

Unless described otherwise, the test substances are in each case administered orally by stomach tube to a group of animals (n=6). Corresponding to an application volume of ml/kg of body weight, the test substances are dissolved in suitable solvent mixtures or suspended in 0.5% Tylose.

A group of animals treated with solvent is used as the control.

### Test Procedure

The present telemetry measurement setup is configured for 24 animals. Each test is recorded under a test number (TYear Month Day).

Each of the instrumented rats living in the setup is assigned its own receiving antenna (1010 Receiver, DSI).

The implanted transmitters can be activated from outside by a built-in magnetic switch. During the runup to the test they are switched to transmit. The signals emitted can be recorded online by a data acquisition system (Dataquest™ A.R.T. for WINDOWS, DSI) and processed as required. The data are saved in a folder that is opened for this in each case, and bears the test number.

In the standard procedure, the following are measured for 10 seconds in each case
Systolic blood pressure (SBP)
Diastolic blood pressure (DBP)
Mean arterial pressure (MAP)
Heart rate (HR)
Activity (ACT)

Recording of the measured values is repeated at 5-minute intervals, under computer control. The source data ascertained as absolute value are corrected in the diagram with the barometric pressure actually measured (Ambient Pressure Reference Monitor; APR-1) and saved as individual data. Further technical details can be found in the extensive documentation from the manufacturer (DSI).

Unless described otherwise, the test substances are administered on the test day 09:00 h. Following application, the parameters described above are measured for 24 hours.

### Evaluation

At the end of test, the individual data obtained are sorted with the analysis software (DATAQUEST™ A.R.T.™ ANALYSIS). Two hours before application is taken as the blank, so that the selected data set covers the period from 07:00 h on the test day to 09:00 h on the next day.

The data are smoothed over a presettable time by mean value determination (15 minutes average) and transferred as a text file to a storage medium. The presorted and compressed measured values are transferred to Excel templates and represented in tabular form. The data obtained are saved per test day in their own file, which bears the test number. Results and test protocols are filed in paper form, sorted by numbers, in folders.

### B-5. In Vivo Test for Detecting Cardiovascular Effects: Diuresis Investigations on Conscious Rats in Metabolism Cages

Wistar rats (200-400 g body weight) are housed with free access to feed (Altromin) and drinking water. During the test, the animals are kept for 4 hours individually in metabolism cages suitable for rats of this weight category (from Tecniplast Deutschland GmbH, D-82383 Hohenpeiβenberg) with free access to drinking water. The test substance is administered in a volume of 1 to 2 ml/kg of body weight of a suitable solvent, orally by means of a stomach tube into the stomach, or is administered intravenously. Animals serving as control receive solvent only, by the corresponding route of application. Controls and substance tests are carried out in parallel on the same day. Control groups and substance dose groups comprise 4 to 8 animals in each case. During the test, urine excreted by the animals is collected continuously in a collecting container at the bottom of the cage. For each animal, the urine volume per unit time is determined separately and the concentration of sodium or potassium ions excreted in the urine is measured by standard methods of flame photometry. In order to obtain a sufficient amount of urine, a defined amount of water is supplied to the animals by stomach tube at the start of the test (typically 10 ml per kg of body weight). The body weight of the individual animals is determined before the start of the test and after the end of the test.

### B-6. In Vivo Test for Detecting Cardiovascular Effects: Effects of Test Substances in Glycerol-Induced Acute Renal Failure

Wistar rats (200-320 g body weight) are housed with free access to feed (Altromin) and drinking water. In each test, with rats of equal age, acute renal failure is induced under light ether narcosis by intramuscular injection of a glycerol-water mixture (mixture ratio by volume 1:1, injection volume 10 ml/kg), in both rear legs. The severity of renal failure can be influenced additionally by the time point (typically 14 to 24 hours) of stopping supply of drinking water before the glycerol injection. The test substance is administered orally by means of a stomach tube into the stomach in a volume of 1 to 2 ml/kg of body weight of a suitable solvent, or it is administered intravenously. Animals serving as control receive solvent only, by the corresponding route of application. Controls and substance tests are carried out in parallel on the same day. Control groups and substance dose groups comprise 8 to 12 animals in each case. In a test, a control group of rats of equal age, which receive ether narcosis and solvent at the same time, but no intramuscular glycerol injection, is investigated simultaneously. After application of the substance or administration of solvent, the rats are kept individually in metabolism cages (from Tecniplast Deutschland GmbH, D-82383 Hohenpeiβenberg). The urine is collected on the 1st and on the 2nd day after glycerol injection, for a period of 24 hours. The contents of sodium, potassium, uric acid and creatinine in the urine are determined Blood for determination of urea, creatinine, uric acid and sodium is taken in each case at the end of the urine collecting period by retroorbital puncture under mild ether narcosis, or by cardiocentesis (48 hours after glycerol injection). Sodium or potassium ions. The determination of sodium and potassium ions is measured by standard methods of flame photometry. Creatinine, urea and uric acid are determined by standard enzymatic and biochemical methods.

B-7. In Vivo Test for Detecting Cardiovascular Effects: Investigations on Rats with Chronic Renal Failure Induced by 5/6 Nephrectomy

The renal-protective action of the test substances is demonstrated in rats with 5/6 nephrectomy (chronic renal failure). These rats are characterized by glomerular hyperfiltration and by development of progressive renal failure, which leads to end-stage kidney diseases and hypertension-induced left ventricular hypertrophy and cardiac fibrosis. Various groups are compared in the experiment: a sham-operated control group, a group with 5/6 nephrectomy and groups with 5/6 nephrectomy treated with test substances. The test substances are applied orally. The renal insufficiency via 5/6 nephrectomy is induced by complete removal of the right kidney and after another two weeks by ligation of the upper and lower third of the remaining kidney. After the second operation, the rats develop progressive renal failure (decrease in GFR) with proteinuria and hypertension. The heart is characterized by uremically hypertensive heart disease. Without treatment, the rats die between week 19 and 26 from end-stage kidney disease or from hypertension-induced terminal organ damage. (Kalk P., Godes M., Relle K., Rothkege C. l, Hucke A., Stasch J. P. and Hocher B.: NO-independent activation of soluble guanylate cyclase prevents disease progression in rats with 5/6 nephrectomy. Brit. J. Pharmacol. 2006, 148, 853-859). For collecting the urine, the animals are kept in metabolism cages for 24 hours. Sodium, potassium, calcium, phosphate and protein are determined. The serum concentrations of glucose, CrP (C-reactive peptide), ALAT (alanine aminotransferase), ASAT (aspartate aminotransferase), potassium, sodium, calcium, phosphate, urea and creatinine are determined with assay kits in automatic analysis equipment. The protein concentrations in the urine and serum are determined using a pyrogallol red-molybdate complex reagent in automatic analysis equipment. The glomerular filtration rate is calculated on the basis of the creatinine clearance. Systolic blood pressure and heart rate are measured by plethysmography with a tail cuff on conscious rats. Body weight is measured weekly. The plasma renin activity and aldosterone in the urine are determined with commercially available radioimmunoassays. All the rats are killed at the end of the study. Blood samples are taken for determination of glucose, creatinine, urea, liver enzymes, CrP, sodium, serum protein and plasma renin activity. Body and heart weight and the weights of the kidneys are measured.

### C. EXAMPLES OF APPLICATION OF PHARMACEUTICAL COMPOSITIONS

The compounds according to the invention can be transformed to pharmaceutical preparations as follows:

### Tablet:

### Composition:

100 mg of the compound according to the invention, 50 mg lactose (monohydrate), 50 mg maize starch (native), 10 mg polyvinylpyrrolidone (PVP 25) (from BASF, Ludwigshafen, Germany) and 2 mg magnesium stearate.

Tablet weight 212 mg. Diameter 8 mm, radius of convexity 12 mm

### Production:

The mixture of compound according to the invention, lactose and starch is granulated in water with a 5% solution (w/w) of PVP. After drying, the granules are mixed with the magnesium stearate for 5 minutes. This mixture is compressed with a usual tablet press (for tablet format, see above). A pressing force of 15 kN is used as a guide value for compression.

### Suspension for Oral Application:

### Composition:

1000 mg of the compound according to the invention, 1000 mg ethanol (96%), 400 mg Rhodigel® (xanthan gum from the company FMC, Pennsylvania, USA) and 99 g water.

10 ml oral suspension corresponds to a single dose of 100 mg of the compound according to the invention.

### Production:

The Rhodigel is suspended in ethanol, the compound according to the invention is added to the suspension. The water is added while stirring. It is stirred for approx. 6 h, until swelling of the Rhodigel ceases.

### Solution for Oral Application:

### Composition:

500 mg of the compound according to the invention, 2.5 g polysorbate and 97 g polyethylene glycol 400.20 g of oral solution corresponds to a single dose of 100 mg of the compound according to the invention.

### Production:

The compound according to the invention is suspended in the mixture of polyethylene glycol and polysorbate, with stirring. The stirring operation is continued until the compound according to the invention has dissolved completely.

i.v. Solution:

The compound according to the invention is dissolved at a concentration below the saturation solubility in a physiologically compatible solvent (e.g. isotonic common salt solution, 5% glucose solution and/or 30% PEG 400 solution). The solution is submitted to sterile filtration and is filled in sterile and pyrogen-free injection containers.

1. A compound of formula (I) 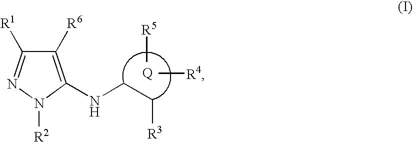
in which
Q stands for phenyl or pyridyl,
R¹ stands for hydrogen, cyano, (C₁-C₃)-alkyl, trifluoromethyl, (C₁-C₃)-alkoxy or trifluoromethoxy,
R² stands for phenyl, naphthyl or 5- or 6-membered heteroaryl,
where phenyl, naphthyl and 5- or 6-membered heteroaryl can be substituted with 1 or 2 substituents selected independently of one another from the group comprising halogen, cyano, (C₁-C₄)-alkyl, monofluoromethyl, difluoromethyl, trifluoromethyl, (C₁-C₄)-alkoxy, monofluoromethoxy, difluoromethoxy and trifluoromethoxy,
R³ stands for hydroxycarbonyl, aminocarbonyl, cyanoaminocarbonyl, (C₁-C₄)-alkylsulfonylaminocarbonyl, oxadiazolonyl or tetrazol-5-yl, where oxadiazolonyl can be substituted with a methyl substituent,
R⁴ stands for hydrogen, halogen, (C₁-C₄)-alkyl, monofluoromethyl, difluoromethyl, trifluoromethyl, (C₁-C₄)-alkoxy, monofluoromethoxy, difluoromethoxy or trifluoromethoxy,
R⁵ stands for hydrogen, halogen, (C₁-C₄)-alkyl, monofluoromethyl, difluoromethyl, trifluoromethyl, (C₁-C₄)-alkoxy, monofluoromethoxy, difluoromethoxy or trifluoromethoxy,
R⁶ stands for a group of formula 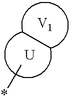
where
* denotes the site of attachment to the pyrazole,
ring U stands for phenyl, pyridyl, pyrimidinyl or pyrazinyl,
in which phenyl, pyridyl, pyrimidinyl and pyrazinyl can be substituted with 1 to 3 substituents selected independently of one another from the group comprising halogen, (C₁-C₄)-alkyl, trifluoromethyl, hydroxy, (C₁-C₄)-alkoxy and trifluoromethoxy,
in which (C₁-C₄)-alkyl and (C₁-C₄)-alkoxy for their part can be substituted with ¹ or ² substituents selected independently of one another from the group comprising hydroxy and (C₁-C₄)-alkoxy,
and
ring V₁ stands for a phenyl ring fused to ring U or a 5- or 6-membered heteroaryl ring fused to ring U,
in which the phenyl ring and the 5- or 6-membered heteroaryl ring can be substituted with ¹ to ⁴ substituents selected independently of one another from the group comprising halogen, cyano, (C₁-C₄)-alkyl, trifluoromethyl, (C₁-C₄)-alkoxy, trifluoromethoxy, (C₁-C₄)-alkylcarbonyl, amino, mono-(C₁-C₄)-alkylamino and di-(C₁-C₄)-alkylamino,
and their salts, solvates and solvates of the salts.

2. The compound of formula (I) as claimed in claim 1, in which
Q stands for phenyl,
R¹ stands for hydrogen, methyl or trifluoromethyl,
R² stands for phenyl,
where phenyl can be substituted with 1 or 2 substituents selected independently of one another from the group comprising fluorine, chlorine, methyl, ethyl, trifluoromethyl, methoxy, ethoxy and trifluoromethoxy,
R³ stands for hydroxycarbonyl,
R⁴ stands for hydrogen, fluorine, chlorine, methyl, ethyl, difluoromethyl, trifluoromethyl, methoxy, ethoxy, difluoromethoxy or trifluoromethoxy,
R⁵ stands for hydrogen,
R⁶ stands for a group of formula 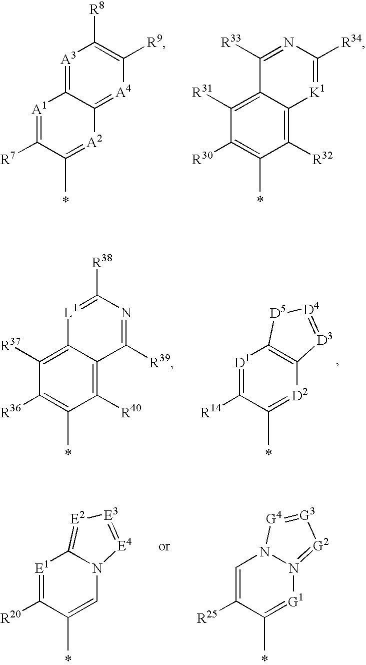
where
* denotes the site of attachment to the pyrazole,
A¹ stands for CR¹⁰ or N,
in which
R¹⁰ stands for hydrogen, fluorine, chlorine or methyl,
A² stands for CR¹¹ or N,
in which
R¹¹ stands for hydrogen, fluorine, chlorine or methyl,
A³ stands for CR¹² or N,
in which
R¹² stands for hydrogen, fluorine, chlorine, methyl or methoxy,
A⁴ stands for CR¹³ or N,
in which
R¹³ stands for hydrogen, fluorine, chlorine, methyl or methoxy,
D¹ stands for CR¹⁵ or N,
in which
R¹⁵ stands for hydrogen, fluorine, chlorine or methyl,
D² stands for CR¹⁶ or N,
in which
R¹⁶ stands for hydrogen, fluorine, chlorine or methyl,
D³ stands for CR¹⁷ or N,
in which
R¹⁷ stands for hydrogen, fluorine, chlorine or methyl,
D⁴ stands for CR¹⁸ or N,
in which
R¹⁸ stands for hydrogen, fluorine, chlorine or methyl,
D⁵ stands for NR¹⁹, O or S,
in which
R¹⁹ stands for hydrogen or methyl,
with the proviso that at least one of the groups D¹, D², D³, D⁴ and D⁵ stands for N or NR¹⁹,
E¹ stands for CR²¹ or N,
in which
R²¹ stands for hydrogen, fluorine, chlorine or methyl,
E² stands for CR²² or N,
in which
R²² stands for hydrogen, fluorine, chlorine or methyl,
E³ stands for CR²³ or N,
in which
R²³ stands for hydrogen, fluorine, chlorine, methyl or amino,
E⁴ stands for CR²⁴ or N,
in which
R²⁴ stands for hydrogen, fluorine, chlorine or methyl,
with the proviso that at most 2 of the groups E², E³ and E⁴ stand for N,
G¹ stands for CR²⁶ or N,
in which
R²⁶ stands for hydrogen, fluorine, chlorine or methyl,
G² stands for CR²⁷ or N,
in which
R²⁷ stands for hydrogen, fluorine, chlorine or methyl,
G³ stands for CR²⁸ or N,
in which
R²⁸ stands for hydrogen, fluorine, chlorine, methyl or amino,
G⁴ stands for CR²⁹ or N,
in which
R²⁹ stands for hydrogen, fluorine, chlorine or methyl,
with the proviso that at most 2 of the groups G², G³ and G⁴ stand for N,
K¹ stands for CR³⁵ or N,
in which
R³⁵ stands for hydrogen, fluorine, chlorine or methyl,
L¹ stands for CR⁴¹ or N,
in which
R⁴¹ stands for hydrogen, fluorine, chlorine or methyl,
R⁷ stands for hydrogen, fluorine, chlorine, methyl, hydroxy, methoxy or ethoxy,
R⁸ stands for hydrogen, fluorine, chlorine, methyl or methoxy,
R⁹ stands for hydrogen, fluorine, chlorine, methyl, methoxy, amino, methylamino or dimethylamino,
R¹⁴ stands for hydrogen, fluorine, chlorine, methyl, hydroxy, methoxy or ethoxy,
R²⁰ stands for hydrogen, fluorine, chlorine, methyl, hydroxy, methoxy or ethoxy,
R²⁵ stands for hydrogen, fluorine, chlorine, methyl, hydroxy, methoxy or ethoxy,
R³⁰ stands for hydrogen, fluorine, chlorine, methyl, hydroxy, methoxy or ethoxy,
R³¹ stands for hydrogen, fluorine, chlorine or methyl,
R³² stands for hydrogen, fluorine, chlorine or methyl,
R³³ stands for hydrogen, fluorine, chlorine, methyl or amino,
R³⁴ stands for hydrogen, fluorine, chlorine, methyl, methoxy, amino, methylamino or dimethylamino,
R³⁶ stands for hydrogen, fluorine, chlorine, methyl, hydroxy, methoxy or ethoxy,
R³⁷ stands for hydrogen, fluorine, chlorine or methyl,
R³⁸ stands for hydrogen, fluorine, chlorine, methyl, methoxy, amino, methylamino or dimethylamino,
R³⁹ stands for hydrogen, fluorine, chlorine, methyl or amino, and
R⁴⁰ stands for hydrogen, fluorine, chlorine or methyl,
and their salts, solvates and solvates of the salts.

3. The compound of formula (I) as claimed in claim 1, in which
Q stands for phenyl,
R¹ stands for hydrogen, cyano, methyl, ethyl or trifluoromethyl,
R² stands for phenyl,
where phenyl can be substituted with 1 or 2 substituents selected independently of one another from the group comprising fluorine, chlorine, (C₁-C₄)-alkyl, trifluoromethyl, (C₁-C₄)-alkoxy and trifluoromethoxy,
R³ stands for hydroxycarbonyl or methylsulfonylaminocarbonyl,
R⁴ stands for hydrogen, fluorine, chlorine, methyl, ethyl, difluoromethyl, trifluoromethyl, methoxy, ethoxy, difluoromethoxy or trifluoromethoxy,
R⁵ stands for hydrogen, fluorine, chlorine, methyl, ethyl, difluoromethyl, trifluoromethyl, methoxy, ethoxy, difluoromethoxy or trifluoromethoxy,
R⁶ stands for a group of formula 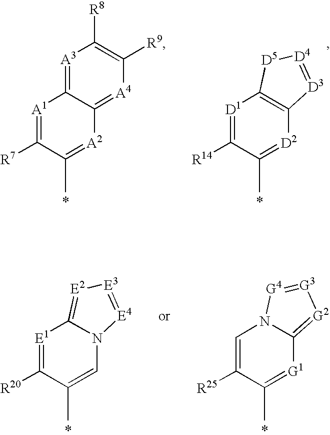
where
* denotes the site of attachment to the pyrazole,
A¹ stands for CR¹⁰ or N,
in which
R¹⁰ stands for hydrogen, fluorine, chlorine or methyl,
A² stands for CR¹¹ or N,
in which
R¹¹ stands for hydrogen, fluorine, chlorine or methyl,
A³ stands for CR¹² or N,
in which
R¹² stands for hydrogen, fluorine, chlorine or methyl,
A⁴ stands for CR¹³ or N,
in which
R¹³ stands for hydrogen, fluorine, chlorine or methyl,
D¹ stands for CR¹⁵ or N,
in which
R¹⁵ stands for hydrogen, fluorine, chlorine or methyl,
D² stands for CR¹⁶ or N,
in which
R¹⁶ stands for hydrogen, fluorine, chlorine or methyl,
D³ stands for CR¹⁷ or N,
in which
R¹⁷ stands for hydrogen, fluorine, chlorine or methyl,
D⁴ stands for CR¹⁸ or N,
in which
R¹⁸ stands for hydrogen, fluorine, chlorine or methyl,
D⁵ stands for NR¹⁹, O or S,
in which
R¹⁹ stands for hydrogen or methyl,
E¹ stands for CR²¹ or N,
in which
R²¹ stands for hydrogen, fluorine, chlorine or methyl,
E² stands for CR²² or N,
in which
R²² stands for hydrogen, fluorine, chlorine or methyl,
E³ stands for CR²³ or N,
in which
R²³ stands for hydrogen, fluorine, chlorine or methyl,
E⁴ stands for CR²⁴ or N,
in which
R²⁴ stands for hydrogen, fluorine, chlorine or methyl,
with the proviso that at most 2 of the groups E², E³ and E⁴ stand for N,
G¹ stands for CR²⁶ or N,
in which
R²⁶ stands for hydrogen, fluorine, chlorine or methyl,
G² stands for CR²⁷ or N,
in which
R²⁷ stands for hydrogen, fluorine, chlorine or methyl,
G³ stands for CR²⁸ or N,
in which
R²⁸ stands for hydrogen, fluorine, chlorine or methyl,
G⁴ stands for CR²⁹ or N,
in which
R²⁹ stands for hydrogen, fluorine, chlorine or methyl,
with the proviso that at most 2 of the groups G², G³ and G⁴ stand for N,
R⁷ stands for hydrogen, fluorine, chlorine or methyl,
R⁸ stands for hydrogen, fluorine, chlorine or methyl,
R⁹ stands for hydrogen, fluorine, chlorine or methyl,
R¹⁴ stands for hydrogen, fluorine, chlorine or methyl,
R²⁰ stands for hydrogen, fluorine, chlorine or methyl,
and
R²⁵ stands for hydrogen, fluorine, chlorine or methyl,
and their salts, solvates and solvates of the salts.

4. The compound of formula (I) as claimed in claim 1, in which
Q stands for a group of formula 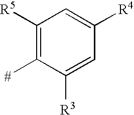
where
# denotes the site of attachment to the amino group,
R³ stands for hydroxycarbonyl,
R⁴ stands for hydrogen, fluorine, chlorine, methyl, ethyl, difluoromethyl, trifluoromethyl, methoxy, difluoromethoxy or trifluoromethoxy,
R⁵ stands for hydrogen or fluorine,
R¹ stands for hydrogen or methyl,
R² stands for phenyl,
where phenyl can be substituted with 1 or 2 substituents selected independently of one another from the group comprising fluorine, chlorine, methyl, ethyl, trifluoromethyl, methoxy, ethoxy or trifluoromethoxy,
R⁶ stands for a group of formula 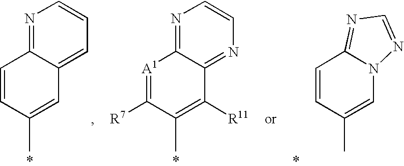
where
* denotes the site of attachment to the pyrazole,
A¹ stands for CR¹⁰ or N,
in which
R¹⁰ stands for hydrogen, fluorine, chlorine or methyl,
R⁷ stands for hydrogen, fluorine, chlorine or methyl,
R¹¹ stands for hydrogen, fluorine, chlorine or methyl,
and their salts, solvates and solvates of the salts.

5. The compound of formula (I) as claimed in claim 1, in which
Q stands for a group of formula 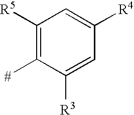
where
# denotes the site of attachment to the amino group,
R³ stands for hydroxycarbonyl,
R⁴ stands for hydrogen, fluorine, chlorine, methyl, ethyl, difluoromethyl, trifluoromethyl, methoxy, difluoromethoxy or trifluoromethoxy,
R⁵ stands for hydrogen,
R¹ stands for methyl,
R² stands for a group of formula 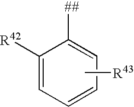
where
## stands for the site of attachment to the pyrazole,
R⁴² stands for hydrogen, fluorine, chlorine, trifluoromethyl, methyl, ethyl, methoxy or ethoxy,
R⁴³ stands for hydrogen, fluorine, chlorine or methyl,
R⁶ stands for a group of formula 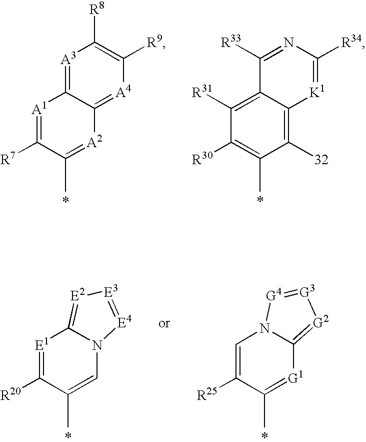
where
* denotes the site of attachment to the pyrazole,
A¹ stands for CR¹⁰ or N,
in which
R¹⁰ stands for hydrogen, fluorine or chlorine,
A² stands for CR¹¹,
in which
R¹¹ stands for hydrogen, fluorine, chlorine or methyl,
A³ stands for N,
A⁴ stands for N,
E¹ stands for CR²¹,
in which
R²¹ stands for hydrogen,
E² stands for N,
E³ stands for CR²³,
in which
R²³ stands for hydrogen or amino,
E⁴ stands for N,
G¹ stands for CR²⁶,
in which
R²⁶ stands for hydrogen,
G² stands for N,
G³ stands for CR²⁸,
in which
R²⁸ stands for hydrogen or amino,
G⁴ stands for N,
K¹ stands for N,
R⁷ stands for hydrogen, fluorine, chlorine, methyl, methoxy or ethoxy,
R⁸ stands for hydrogen,
R⁹ stands for hydrogen,
R²⁰ stands for hydrogen, fluorine, chlorine, methyl, methoxy or ethoxy,
R²⁵ stands for hydrogen, fluorine, chlorine, methyl, methoxy or ethoxy,
R³⁰ stands for hydrogen, fluorine, chlorine, methyl, methoxy or ethoxy,
R³¹ stands for hydrogen, fluorine or chlorine,
R³² stands for hydrogen, fluorine or chlorine,
R³³ stands for hydrogen,
and
R³⁴ stands for hydrogen,
and their salts, solvates and solvates of the salts.

6. A method of production of the compound of formula (I), as defined in claim 1, characterized in that
[A] a compound of formula (II) 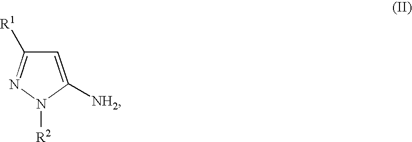
in which R¹ and R² in each case have the meanings given in claim 1,
is transformed in an inert solvent with a halogenating agent to a compound of formula (III-A) 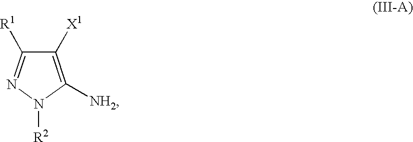
in which R¹ and R² in each case have the meanings given in claim 1,
and
X¹ stands for halogen, in particular for bromine or iodine, this is then reacted in an inert solvent in the presence of a base and a suitable palladium catalyst with a compound of formula (IV) 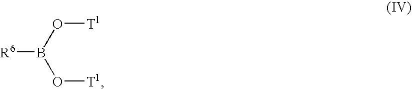
in which R⁶ has the meaning given in claim 1
and
T¹ stands for hydrogen or both residues T¹ together form a C(CH³)²C(CH³)²— or —CH²C(CH³)²CH²— bridge,
to a compound of formula (V-A) 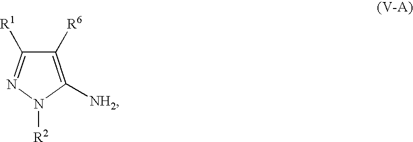
in which R¹, R² and R⁶ in each case the have the meanings given in claim 1,
and this is then reacted in an inert solvent in the presence of a suitable catalyst with a compound of formula (VI-A) 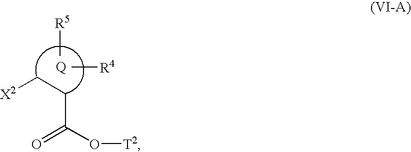
in which Q, R⁴ and R⁵ in each case the have the meanings given in claim 1
and
T² stands for (C₁-C₄)-alkyl,
X² stands for halogen, preferably bromine,
to a compound of formula (VII) 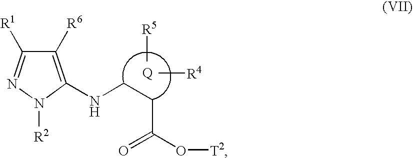
in which Q, T², R¹, R², R⁴, R⁵ and R⁶ in each case have the meanings stated previously,
or
[B] a compound of formula (II) is reacted in an inert solvent in the presence of a suitable catalyst with a compound of formula (VI-A) to a compound of formula (III B) 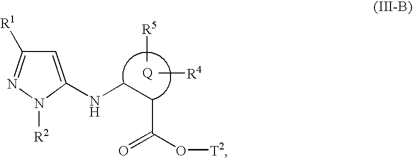
in which Q, R¹, R², R⁴ and R⁵ in each case the have the meanings given in claim 1,
and
T² stands for (C₁-C₄)-alkyl, and this is then transformed in an inert solvent with a halogenating agent to a compound of formula (V-B) 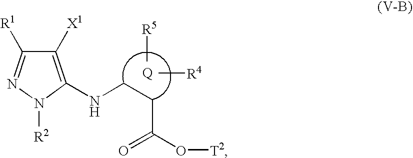
in which Q, T², X¹, R¹, R², R⁴ and R⁵ in each case have the meanings stated previously,
and
X¹ stands for halogen, preferably bromine,
and this is then reacted in an inert solvent in the presence of a base and a suitable palladium catalyst with a compound of formula (IV) to a compound of formula (VII),
or
[C] a compound of formula (VIII)
R⁶—X³ (VIII),
in which R⁶ has the meaning given in claim 1
and
X³ stands for halogen, preferably bromine or iodine, is reacted in an inert solvent in the presence of a suitable palladium catalyst with trimethylsilylacetonitrile to a compound of formula (IX) 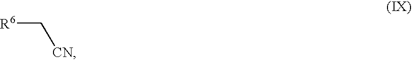
in which R⁶ has the meaning given in claims 1 to 5,
and this is then reacted in an inert solvent in the presence of a suitable base with an ester of formula (X) 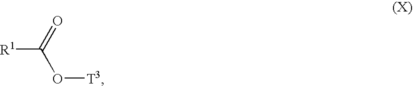
in which R¹ has the meaning given in claim 1
and
T³ stands for (C₁-C₄)-alkyl,
to a compound of formula (XI) 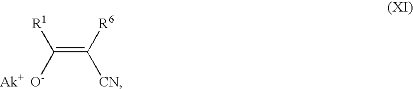
in which R¹ and R⁶ in each case the have the meanings given in claim 1
and
Ak+ stands for an alkali ion, preferably sodium,
and this is then transformed with a hydrazine of formula (XII) 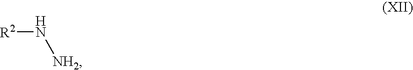
in which R² has the meaning given in claim 1,
to a compound of formula (V-A), and this is reacted further according to method [A] described above to a compound of formula (VII),
and the compound of formula (VII) that results in each case is then transformed by hydrolysis of the ester to a carboxylic acid of formula (I-1) 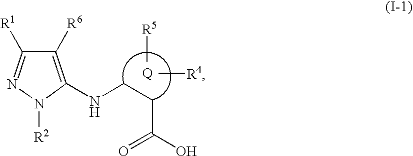
in which Q, R¹, R², R⁴, R⁵ and R⁶ in each case the have the meanings given in claim 1,
and the resultant compounds of formula (I-1) are optionally transformed with the corresponding (i) solvents and/or (ii) bases or acids to their solvates, salts and/or solvates of the salts.

7. A method for the treatment or prophylaxis of a disease comprising the step of administering an effective amount of the compound of formula (I) as defined in claim 1 to a patient in need thereof.

8. The method of claim 7, in which the disease is of acute decompensated and chronic heart failure, hypervolemic and euvolemic hyponatremia, hepatic cirrhosis, ascites, edemas, nephropathy, acute and chronic renal failure, renal insufficiency or the syndrome of inappropriate secretion of antidiuretic hormone (SIADH).

9. (canceled)

10. A medicinal product comprising a compound of formula (I), as defined in claim 1, in combination with an inert, nontoxic, pharmaceutically suitable excipient.

11. The medicinal product as claimed in claim 10, in combination with one or more diuretics, angiotensin AII antagonists, ACE inhibitors, beta-receptor blockers, mineralocorticoid receptor antagonists, organic nitrates, NO donors, guanylate cyclase stimulators, guanylate cyclase activators, vasopressin antagonists, or substances with positive inotropic action.

12. (canceled)

13. (canceled)
